SEQUENCE LISTING (168 sequences)

SEQ ID NO: 1 - protein, 115 aa, synthetic construct
SGGGLVQPGGSLRLSCAASGSIFSIYAMGWYRQAPGKQRELVAAISSYGSTNYADSVKGRFTISRDNAKNTVYLQMNSLKPEDTAVYYCNADIATMTAVGGFDYWGQGTQVTVSS

SEQ ID NO: 2 - protein, 111 aa, synthetic construct
SGGGSVQPGGSLRLSCAAIGSVFTMYTTAWYRQTPGNLRELVASITDEHRTNYAASAEGRFTISRDNAKHTVDLQMTNLKPEDTAVYYCKLEHDLGYYDYWGQGTQVTVSS

SEQ ID NO: 3 - protein, 119 aa, synthetic construct
SGGGLVQPGGSLRLSCATSGFTLEYYAIGWFRQAPGKGREGVACMNSSGGGTNYADSVKGRFTISRDNAKKMVYLQMNSLKSEDTAVYYCVVDDFRCGSRWAAYLRSSWGQGTQVTVSS

SEQ ID NO: 4 - protein, 118 aa, synthetic construct
SGGGLVQPGGSLRLSCVVSGSDFNTYIMGWYRQVPGKPRELVADITTEGKTNYGGSVKGRFTISRDNAKNTVYLQMFGLKPEDAGNYVCNADWKMGAWTAGDYGIDYWGKGTLVTVSS

SEQ ID NO: 5 - protein, 114 aa, synthetic construct
SGGGLVQPGGSLRLSCAASGFTLGSRYMSWVRQAPGEGFEWVSSIEPSGTAWDGDSAKGRFTTSRDDAKNTLYLQMSNLQPEDTGVYYCATGYRTDTRIPGGSWGQGTQVTVSS

SEQ ID NO: 6 - protein, 108 aa, synthetic construct
SGGGLVQVGGSLRLSCVVSGSDISGIAMGWYRQAPGKRREMVADIFSGGSTDYAGSVKGRFTISRDNAKKTSYLQMNNVKPEDTGVYYCRLYGSGDYWGQGTQVTVSS

SEQ ID NO: 7 - protein, 118 aa, synthetic construct
SGGGLVQPGGSLRLSCAASGFTLDDYAIGWFRQVPGKEDEGVSCMSRSGDTYYPHSVKGRFTISVDNAKNTMYLQMNNLKPEDTAVYYCAIDFPPVRPMCIQAAPKKRSRGTQVTVSA

SEQ ID NO: 8 - protein, 116 aa, synthetic construct
TGGGLVQAGGSLRLSCTASGADFSFYAMGWYRQTPGNSRELVAVMNLNGVISYGDSARGRFDISRDGTKNIVFLQMNSLKPEDTGVYYCNGMRLYTRGSVRHPESWGQGIQVTVSS

SEQ ID NO: 9 - protein, 124 aa, synthetic construct
TGGGLVQAGDSLTLSCAATGRTLDYYALGWFRQVPGNKREFVAAINWLGGSTYYADSVRGRFTLSRDNSKSTLYLNMNNLIPDDTAVYYCAADFSIAYSGTYPPAYAEYDYDYWGQGTQVTVSS

SEQ ID NO: 10 - protein, 115 aa, synthetic construct
SGGLVQPGGSLKLSCAHSGSPLSIWVMGWYRQAPGKQRELVALINLNGITSYGDSVKGRFTISRDYAENTAYLQMNSLKFEDTAVYYCNAEPLGPRGKKSGKEYWGTGTQVTVSL

SEQ ID NO: 11 - protein, 105 aa, synthetic construct
TGGALVQPGQSLTLSCTTSENVFGIYGMAWLRQAPGRQRELVASITSRGTAHYHDSVKGRFTISRESGKTTAYLQTTSVNPEDTAIYYCNSGPYWGQGTQVTVSS

SEQ ID NO: 12 - protein, 111 aa, synthetic construct
SGGGLVQPGGSLRLSCAASGFTFNRYVIRWYRQAPGKERELVAGISRSGDSGRYVDSVKGRFTISRDNDKNMAYLQMSSLKPDDTAVYYCSALNLEDMEYWGQGTQVTVSS

SEQ ID NO: 13 - protein, 116 aa, synthetic construct
SGGGLVQPGGSLRLSCAASGNIFSIYYMGWYRQAPGKQREMVAIINSNGITNYGDFVKGRFTISRDNAENSAYLQMNNLTPEDTAVYYCNAGKLRRTTGWGLDDYWGQGTQVTVSS

SEQ ID NO: 14 - protein, 119 aa, synthetic construct
SGGGTVQPGGTLRLSCAASGFTLDEYAIGWFRQAPGKEREGVSCISSSASISYADSVKGRFTISRDNAKNTVYLTMNSLKPEDTGVYYCARAFLACGPVAGWGTEYDYWGQGTQVTVSS

SEQ ID NO: 15 - protein, 115 aa, synthetic construct
TGGGLVQPGGSLRLSCTASTTISDFYSMGWFRQTPGNQRELVAIVRRGGDTKSGDSVKGRFTISRDNTRSTVYLQMDNLKPEDTAVYYCYANLQKSSDELGPYYWGQGTQVTVSS

SEQ ID NO: 16 - protein, 119 aa, synthetic construct
SGGGLVQSGGSLRLSCAASLLTLEYYAIGWFRQAPGKEREGVSCTGSSGGSTVYIDSVKGRFTVVRDNAKNMVYLQMDNLQPEDTAVYYCAADDLRCGRGWSSYFRGSWGQETQVTVSS

SEQ ID NO: 17 - protein, 111 aa, synthetic construct
TGGGLVQPGGSLRLACVASESVFEMYTVAWYRQAPGKQRELVAGITDEGRTNYADFVKGRFTISRDNSKKTVHLQMDNLNPEDTAVYYCKLEHDLGYYDYWGQGTQVTVSS

SEQ ID NO: 18 - protein, 121 aa, synthetic construct
SGGGLVQPGGSLRLSCAASGLTLDYYAIGWFRQAPGKEREGVSCISSGSSMSIHADSVKGRFTISRDNAKNTVYLQMNSLKPEDTAVYYCAADDFTCGSRWSDWAHTFGFWGQGTQVTVSS

SEQ ID NO: 19 - protein, 116 aa, synthetic construct
SGGLVQPGGSLTLSCVVSGGIFSTYIMGWYRQVPGRQREMVATISNHTTDYADFVQGRFTISRDIAKKAVYLQMHSLKPDDTGRYVCNADWMVGAWTAGDYGVDYWGKGILVTVSS

SEQ ID NO: 20 - protein, 121 aa, synthetic construct
SGGGMVQPGGSLRLSCAASGFTFSTYDMSWVRQAPGKGPEWVSIINAGGGSTYYAASVKGRFAISRDNAKNTLYLQMNNLKPEDTALYYCARVASYYCRGYVCSPPEFDYWGQGTQVTVSS

SEQ ID NO: 21 - protein, 119 aa, synthetic construct
SGGGLVQAGGSLRLSCAPSRLTLDFFAIAWFRQAPGKEREGVSCISSHDGSTYYTDSVKGRFTISKDNAKNTVYLQMNSLKPEDTAVYYCALDHNVGTCQLTQAEYDYWGQGTQVTVSS

SEQ ID NO: 22 - protein, 111 aa, synthetic construct
SGGGLVQSGGSLRLSCAASGSIDSLYHMGWYRQAPGKERELVARVQDGGSTAYKDSVKGRFTISRDFSRSTMYLQMNSLKPEDTAIYYCAAKSTISTPLSWGQGTQVTVSS

SEQ ID NO: 23 - protein, 112 aa, synthetic construct
SGGGLVQPGGSLRLSCAASGFPFHAYYMSWVRQAPGKGLEWVSHIGNGGIITRYADSVKGRFTISRDNAKNTLYLQMTNLKPEDTALYYCTLGTRDDLGPERGQGTQVTVSS

SEQ ID NO: 24 - protein, 123 aa, synthetic construct
TGGGLVQAGGSLGLSCAASGLSFNWYDVGWFRQAPGKEREFVASRSSGGGSTYYGDSVKGRFSISTDNAKNTAYLQMNSLKPEDTAVYYCAADWTGRAGFSVGYYRPDEYDYWGQGTQVTVSE

SEQ ID NO: 25 - protein, 120 aa, synthetic construct
TGGGLVQPGGSLRLSCVASGFTLDSYAIGWFRQAPGKEREGVSCMSSGDGSTYYTNSVKGRFTISRDNAQNTVYLQMNSLKPEDTAVYYCAADGFDYCSAYVPGRGMNYSGKGTLVTVSS

SEQ ID NO: 26 - protein, 121 aa, synthetic construct
TGGGLVQPGGSLRLSCAGSGFTLDNYAVGWFRQAPGKEREGVSCISSSDDNTDYSDSVKGRFTISRDNAKDTVYLQMNSLKPEDTAIYYCAAESPTFGFSCTVATDPYDYWGQGTQVTVSS

SEQ ID NO: 27 - protein, 116 aa, synthetic construct
TGGGLVQPGGSLRLSCAASGFTLDGYAAGWFRQAPGKERELVSWISSTDGSTYYAASVKGRFTVSRDNAKNTVYLQMNSLKPEDTAVYYCTAGLGLDVSDYVYDYWGQGTQVTVSS

SEQ ID NO: 28 - protein, 114 aa, synthetic construct
SGGLVQPGGSLRLSCAASGFTLDYYGIGWVRQAPGKEREEVSCITSGGLTNYPDSVKGRFTISRDNAKNTVYLQMNSLKPEDTAVYYCAIDRVGVCAMEDFGSWGQGTQVTVSS

SEQ ID NO: 29 - protein, 118 aa, synthetic construct
TGGGLVQAGDSLRLSCAASGRTFNYYAMAWFRQAPGKEREFVAFINWSGDSTYYAGSVKGRFTISRDNAKNTVYLQMNNLKPEDTAVYSCAAEFGTFSYLQGDDYSYWGQGTQVTVSS

SEQ ID NO: 30 - protein, 117 aa, synthetic construct
SGGGLVQAGGSLRLSCAASGRSFSSYRMGWFRQAPGKERELVAGISWSGSSTWYADSVKGRFTISRDNAKNTVYLQMNSLKPEDTAVYYCAADGLGTDWSDAIWDYWGQGTQVTVSS

SEQ ID NO: 31 - protein, 119 aa, synthetic construct
SGGGLVQAGGSLRLSCAVSGRMFNEYRMGWFRQAPGKEREFVSAINWGAQIPYYADSVKGRFTISRDSAENTVYLQMNSLKPEDTAVYYCAADWGYGSSPHQDKEYDYWGQGTQVTVSS

SEQ ID NO: 32 - protein, 117 aa, synthetic construct
SGGGLVQAGGSLRLSCAASGRTFSDYAMGWFRQAPGKERVFVAAVDWSGSSRLYAESVEGRFTISRDNSKNTVYLQMNTLKPEDTAVYYCAAARNRWSSEISSYDYWGQGTQVTVSS

SEQ ID NO: 33 - protein, 123 aa, synthetic construct
SGGGLVQTGGSLRLSCAASGRTFRRNTMGWFRQAPGKVREFVAAISWSGDRTYCADSVKGRFTISRDNAKNTVDLLMNSLKPEDTAIYYCAADGTASVFNSYASADRNKYNYWGQGTQVTVSS

SEQ ID NO: 34 - protein, 116 aa, synthetic construct
SGGGLVQAGDSLRLSCAASIRTFSTSTTAWFRQAPGKEREFVARISGSDPVTYYTDSVRGRFTISRDNAKSTAYLQMNSLKPEDTGVYYCATVRIKGGSEFSYHYWGQGTQVTVSS

SEQ ID NO: 35 - protein, 116 aa, synthetic construct
TGGGLVQAGGSLRLSCAASESTFSINAIGWYRQAPGKQRELVAHISTSGRTRYADSVKGRFTISRDNAWNTVFLQMISLKPEDTAVYYCNAEGYSTWPEDRYLELWGQGTQVTVSP

SEQ ID NO: 36 - protein, 119 aa, synthetic construct
TGGGLVQAGGSLRLSCTASGRTFSSYRMGMGWFRQAPGKEREFVATVNWSGGTTYYADSVKGRFTISRDNAKNTVYLQMNSLKPEDTAVYYCAAGRGSESYTSSRYNYWGQGTQVTVSS

SEQ ID NO: 37 - protein, 118 aa, synthetic construct
SGGGLVQPGGSLRLSCAASGFTLDDYAIGWFRQAPGKEREAVSCISIRDGRTHYADSVKGRFTISRDNAKNTVYLQMNSLKPEDTAVYYCAAGQRSMAYVCSNRFGSWGQGTQVTVSS

SEQ ID NO: 38 - protein, 13 aa, synthetic construct
SGFTLDDYAIGWF

SEQ ID NO: 39 - protein, 9 aa, synthetic construct
SRSGDTYYP

SEQ ID NO: 40 - protein, 17 aa, synthetic construct
DFPPVRPMCIQAAPKKR

SEQ ID NO: 41 - protein, 13 aa, synthetic construct
SGADFSFYAMGWY

SEQ ID NO: 42 - protein, 9 aa, synthetic construct
NLNGVISYG

SEQ ID NO: 43 - protein, 15 aa, synthetic construct
MRLYTRGSVRHPESW

SEQ ID NO: 44 - protein, 13 aa, synthetic construct
TGRTLDYYALGWF

SEQ ID NO: 45 - protein, 10 aa, synthetic construct
NWLGGSTYYA

SEQ ID NO: 46 - protein, 22 aa, synthetic construct
DFSIAYSGTYPPAYAEYDYDYW

SEQ ID NO: 47 - protein, 13 aa, synthetic construct
SGSPLSIWVMGWY

SEQ ID NO: 48 - protein, 9 aa, synthetic construct
NLNGITSYG

SEQ ID NO: 49 - protein, 15 aa, synthetic construct
EPLGPRGKKSGKEYW

SEQ ID NO: 50 - protein, 13 aa, synthetic construct
TSENVFGIYGMAW

SEQ ID NO: 51 - protein, 10 aa, synthetic construct
ITSRGTAHYH

SEQ ID NO: 52 - protein, 4 aa, synthetic construct
GPYW

SEQ ID NO: 53 - protein, 13 aa, synthetic construct
SGFTFNRYVIRWY

SEQ ID NO: 54 - protein, 10 aa, synthetic construct
SRSGDSGRYV

SEQ ID NO: 55 - protein, 9 aa, synthetic construct
LNLEDMEYW

SEQ ID NO: 56 - protein, 13 aa, synthetic construct
SGNIFSIYYMGWY

SEQ ID NO: 57 - protein, 9 aa, synthetic construct
NSNGITNYG

SEQ ID NO: 58 - protein, 15 aa, synthetic construct
GKLRRTTGWGLDDYW

SEQ ID NO: 59 - protein, 13 aa, synthetic construct
SGFTLDEYAIGWF

SEQ ID NO: 60 - protein, 9 aa, synthetic construct
SSSASISYA

SEQ ID NO: 61 - protein, 18 aa, synthetic construct
AFLACGPVAGWGTEYDYW

SEQ ID NO: 62 - protein, 13 aa, synthetic construct
STTISDFYSMGWF

SEQ ID NO: 63 - protein, 9 aa, synthetic construct
RRGGDTKSG

SEQ ID NO: 64 - protein, 14 aa, synthetic construct
NLQKSSDELGPYYW

SEQ ID NO: 65 - protein, 13 aa, synthetic construct
SLLTLEYYAIGWF

SEQ ID NO: 66 - protein, 10 aa, synthetic construct
GSSGGSTVYI

SEQ ID NO: 67 - protein, 17 aa, synthetic construct
DDLRCGRGWSSYFRGSW

SEQ ID NO: 68 - protein, 13 aa, synthetic construct
SESVFEMYTVAWY

SEQ ID NO: 69 - protein, 9 aa, synthetic construct
TDEGRTNYA

SEQ ID NO: 70 - protein, 10 aa, synthetic construct
EHDLGYYDYW

SEQ ID NO: 71 - protein, 13 aa, synthetic construct
SGLTLDYYAIGWF

SEQ ID NO: 72 - protein, 10 aa, synthetic construct
SSGSSMSIHA

SEQ ID NO: 73 - protein, 19 aa, synthetic construct
DDFTCGSRWSDWAHTFGFW

SEQ ID NO: 74 - protein, 13 aa, synthetic construct
SGGIFSTYIMGWY

SEQ ID NO: 75 - protein, 8 aa, synthetic construct
SNHTTDYA

SEQ ID NO: 76 - protein, 17 aa, synthetic construct
DWMVGAWTAGDYGVDYW

SEQ ID NO: 77 - protein, 11 aa, synthetic construct
SGLSFNWYDVG

SEQ ID NO: 78 - protein, 10 aa, synthetic construct
SRSSGGGSTY

SEQ ID NO: 79 - protein, 22 aa, synthetic construct
AADWTGRAGFSVGYYRPDEYDY

SEQ ID NO: 80 - protein, 11 aa, synthetic construct
SGFTLDSYAIG

SEQ ID NO: 81 - protein, 10 aa, synthetic construct
CMSSGDGSTY

SEQ ID NO: 82 - protein, 19 aa, synthetic construct
AADGFDYCSAYVPGRGMNY

SEQ ID NO: 83 - protein, 11 aa, synthetic construct
SGFTLDNYAVG

SEQ ID NO: 84 - protein, 10 aa, synthetic construct
CISSSDDNTD

SEQ ID NO: 85 - protein, 20 aa, synthetic construct
AAESPTFGFSCTVATDPYDY

SEQ ID NO: 86 - protein, 11 aa, synthetic construct
SGFTLDGYAAG

SEQ ID NO: 87 - protein, 10 aa, synthetic construct
WISSTDGSTY

SEQ ID NO: 88 - protein, 15 aa, synthetic construct
TAGLGLDVSDYVYDY

SEQ ID NO: 89 - protein, 11 aa, synthetic construct
SGFTLDYYGIG

SEQ ID NO: 90 - protein, 9 aa, synthetic construct
CITSGGLTN

SEQ ID NO: 91 - protein, 15 aa, synthetic construct
AIDRVGVCAMEDFGS

SEQ ID NO: 92 - protein, 11 aa, synthetic construct
SGRTFNYYAMA

SEQ ID NO: 93 - protein, 10 aa, synthetic construct
FINWSGDSTY

SEQ ID NO: 94 - protein, 17 aa, synthetic construct
AAEFGTFSYLQGDDYSY

SEQ ID NO: 95 - protein, 11 aa, synthetic construct
SGRSFSSYRMG

SEQ ID NO: 96 - protein, 10 aa, synthetic construct
GISWSGSSTW

SEQ ID NO: 97 - protein, 16 aa, synthetic construct
AADGLGTDWSDAIWDY

SEQ ID NO: 98 - protein, 11 aa, synthetic construct
SGRMFNEYRMG

SEQ ID NO: 99 - protein, 10 aa, synthetic construct
AINWGAQIPY

SEQ ID NO: 100 - protein, 18 aa, synthetic construct
AADWGYGSSPHQDKEYDY

SEQ ID NO: 101 - protein, 11 aa, synthetic construct
SGRTFSDYAMG

SEQ ID NO: 102 - protein, 10 aa, synthetic construct
AVDWSGSSRL

SEQ ID NO: 103 - protein, 16 aa, synthetic construct
AAARNRWSSEISSYDY

SEQ ID NO: 104 - protein, 11 aa, synthetic construct
SGRTFRRNTMG

SEQ ID NO: 105 - protein, 10 aa, synthetic construct
AISWSGDRTY

SEQ ID NO: 106 - protein, 22 aa, synthetic construct
AADGTASVFNSYASADRNKYNY

SEQ ID NO: 107 - protein, 11 aa, synthetic construct
SIRTFSTSTTA

SEQ ID NO: 108 - protein, 10 aa, synthetic construct
RISGSDPVTY

SEQ ID NO: 109 - protein, 15 aa, synthetic construct
ATVRIKGGSEFSYHY

SEQ ID NO: 110 - protein, 11 aa, synthetic construct
SESTFSINAIG

SEQ ID NO: 111 - protein, 9 aa, synthetic construct
HISTSGRTR

SEQ ID NO: 112 - protein, 16 aa, synthetic construct
NAEGYSTWPEDRYLEL

SEQ ID NO: 113 - protein, 13 aa, synthetic construct
SGRTFSSYRMGMG

SEQ ID NO: 114 - protein, 10 aa, synthetic construct
TVNWSGGTTY

SEQ ID NO: 115 - protein, 16 aa, synthetic construct
AAGRGSESYTSSRYNY

SEQ ID NO: 116 - protein, 11 aa, synthetic construct
SGFTLDDYAIG

SEQ ID NO: 117 - protein, 10 aa, synthetic construct
CISIRDGRTH

SEQ ID NO: 118 - protein, 17 aa, synthetic construct
AAGQRSMAYVCSNRFGS

SEQ ID NO: 119 - protein, 10 aa, unidentified
DELGPRLMGK

SEQ ID NO: 120 - protein, 13 aa, unidentified
GAPVPYPDPLEPR

SEQ ID NO: 121 - protein, 5 aa, synthetic construct
GGGGS

SEQ ID NO: 122 - protein, 15 aa, synthetic construct
GGGGSGGGGSGGGGS

SEQ ID NO: 123 - protein, 34 aa, unidentified
EPKTPKPQGGGGSGGGGSGGGGSQGVQSQVQLVE

SEQ ID NO: 124 - protein, 8 aa, unidentified
EPKTPKPQ

SEQ ID NO: 125 - protein, 1296 aa, Clostridium botulinum [sequence omitted]

SEQ ID NO: 126 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 127 - protein, 8 aa, unidentified
DYKDDDDK

SEQ ID NO: 128 - protein, 10 aa, synthetic construct
  VARIANT 1..10: note = This sequence may encompass 5-10 residues
HHHHHHHHHH

SEQ ID NO: 129 - protein, 6 aa, synthetic construct
HHHHHH

SEQ ID NO: 130 - protein, 20 aa, synthetic construct
YSTDLRMTSRYKLKKLKMIF

SEQ ID NO: 131 - protein, 20 aa, synthetic construct
YSTGLRMSFKYKFAEFRVGP

SEQ ID NO: 132 - protein, 20 aa, synthetic construct
YSTDLRMISRYKLKELKLIF

SEQ ID NO: 133 - protein, 20 aa, synthetic construct
YSTELRMTSRYKLKKLKMIF

SEQ ID NO: 134 - protein, 20 aa, synthetic construct
YSTDLRMTSRYKLRKLKMIF

SEQ ID NO: 135 - protein, 19 aa, synthetic construct
DLMENRDKLDKYKMEINKF

SEQ ID NO: 136 - protein, 19 aa, synthetic construct
DLMENRDKLDKYKMEINNF

SEQ ID NO: 137 - protein, 19 aa, synthetic construct
GLMQKSNKAKEYRVRGENP

SEQ ID NO: 138 - protein, 19 aa, synthetic construct
DLMKKSDRLDEYKLEINKF

SEQ ID NO: 139 - protein, 19 aa, synthetic construct
ELMENRDKLDKYKMEINKF

SEQ ID NO: 140 - protein, 21 aa, synthetic construct
MNHNKYRTVRITASQYNVKEL

SEQ ID NO: 141 - protein, 21 aa, synthetic construct
MNHEGYRSVKITTPQYNIIRF

SEQ ID NO: 142 - protein, 21 aa, synthetic construct
MNHNKYRIVRITASQYNVKEL

SEQ ID NO: 143 - protein, 30 aa, synthetic construct
PFNKVDKHNYRLTVRIRSETTASQYMNVKE

SEQ ID NO: 144 - protein, 30 aa, synthetic construct
PFNKVDKHEYRLSVKIRSETTTPQYMNIIR

SEQ ID NO: 145 - protein, 30 aa, synthetic construct
PFNKVDKHNYRLIVRIRSETTASQYMNVKE

SEQ ID NO: 146 - protein, 30 aa, synthetic construct
LFNKVDKHNYRLTVRIRSETTASQYMNVKE

SEQ ID NO: 147 - protein, 30 aa, synthetic construct
PFNKIDKHNYRLTVRIRSETTASQYMNVKE

SEQ ID NO: 148 - protein, 27 aa, synthetic construct
NAQQTTNPEGDNKSFGHEVLNLTRYRP

SEQ ID NO: 149 - protein, 27 aa, synthetic construct
NAQQTTNPEGDNKSFGHDVLNLTRYRP

SEQ ID NO: 150 - protein, 27 aa, synthetic construct
NAQQTTNPEGDNKSFGHDVFNLTRYRP

SEQ ID NO: 151 - protein, 27 aa, synthetic construct
NAKQITNPEVDNSSFRDDVLNLTRYRT

SEQ ID NO: 152 - protein, 27 aa, synthetic construct
NAQQITNPEGDNKSFGHDVLNLTRYRP

SEQ ID NO: 153 - protein, 27 aa, synthetic construct
NAQQTTNPKGDNKSFGHDVLNLTRYRP

SEQ ID NO: 154 - protein, 30 aa, synthetic construct
PNAFTNPEEGGSTDTKSFGHEVLNLTRYRP

SEQ ID NO: 155 - protein, 30 aa, synthetic construct
PNAFTNPEEGGSTDTKSFGHDVLNLTRYRP

SEQ ID NO: 156 - protein, 30 aa, synthetic construct
PNAFTNPEEGGSTDTKSFGHDVFNLTRYRP

SEQ ID NO: 157 - protein, 29 aa, synthetic construct
NAFTNPEEGGGKDTSSFRDDVLNLTRYRT

SEQ ID NO: 158 - protein, 30 aa, synthetic construct
PNAFTNPKEGGSTDTKSFGHDVLNLTRYRP

SEQ ID NO: 159 - protein, 34 aa, synthetic construct
PEAKQVPVQFPVYMSGLEVSERYNFDGLFEFYLL

SEQ ID NO: 160 - protein, 34 aa, synthetic construct
PEAKQVPIESTVYMAGLEVSERYNFDGLFEFYLL

SEQ ID NO: 161 - protein, 34 aa, synthetic construct
PEAKQVPVNFPVYMSGLEVSERYNFDGLFEFYLL

SEQ ID NO: 162 - protein, 31 aa, synthetic construct
PEAKQVPVYDIQFFTPVKEMSGLEVSERYFD

SEQ ID NO: 163 - protein, 31 aa, synthetic construct
PEAKQVPIYNIESVATVKEMAGLEVSERYFD

SEQ ID NO: 164 - protein, 31 aa, synthetic construct
PEAKQVPVYDINFFTPVKEMSGLEVSERYFD

SEQ ID NO: 165 - protein, 35 aa, synthetic construct
FYFGYKDRDEYPVEEIDGQNHFREKVDDPVDAIQE

SEQ ID NO: 166 - protein, 35 aa, synthetic construct
FYFGYKDRDEYPVEEIDGQNHFREKVDDPVDTIQE

SEQ ID NO: 167 - protein, 35 aa, synthetic construct
FYFGYKDRDEYPVEEIDGQNHFREKVNDPVDAIQE

SEQ ID NO: 168 - protein, 16 aa, synthetic construct
RVPEENNILVCISPNN

(19) United States
(12) Patent Application Publication
(10) Pub. No.: US 2026/0184813 A1
(43) Pub. Date: Jul. 2, 2026

(54) VHH POLYPEPTIDES THAT BIND TO LIGHT CHAIN (LC) PROTEASES OF BOTULINUM NEUROTOXINS (BoNTs) AND METHODS OF USE THEREOF

(71) Applicant: Trustees of Tufts College, Medford, MA (US)

(72) Inventor: Charles B. SHOEMAKER, Medford, MA (US)

(73) Assignee: Trustees of Tufts College, Medford, MA (US)

(21) Appl. No.: 17/980,791

(22) Filed: Nov. 4, 2022

Related U.S. Application Data
(60) Provisional application No. 63/275,563, filed on Nov. 4, 2021.

(51) Int. Cl. C07K 16/40; A61K 47/68; A61P 39/02; G01N 33/573

(52) U.S. Cl. CPC C07K 16/40; A61K 47/6893; A61P 39/02; G01N 33/573; C07K 2317/22; C07K 2317/569; G01N 2333/952

(57) ABSTRACT

Products, pharmaceutical compositions, methods of use, and kits are provided for treating a patient suffering from botulinum neurotoxin intoxication. Featured are single-domain variable heavy-chain (VHH) polypeptides (antibodies) that target and neutralize the light chain (LC) protease domains of botulinum neurotoxin (BoNT), i.e., BoNT LC/A and BoNT LC/B, and that advantageously provide late and post-exposure therapy for botulism patients. The LC/A toxin-binding and LC/B toxin-binding VHH polypeptides, which can be recombinantly produced, specifically bind to the LCs of the botulinum neurotoxins to inhibit toxin activity and treat intoxication.

### CROSS REFERENCE TO RELATED APPLICATIONS

This application claims priority to and benefit of U.S. Provisional Application No. 63/275,563, filed on Nov. 4, 2021, the contents of which are hereby incorporated by reference in their entirety.

### STATEMENT OF RIGHTS TO INVENTIONS MADE UNDER FEDERALLY SPONSORED RESEARCH

This invention was made with government support under Grant No. R01AI093467 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Sep. 25, 2023, is named 167774-012702_US_SL.xml and is 154,536 bytes in size.

### BACKGROUND

Botulinum neurotoxins (BoNTs) are extremely potent toxins to humans and cause flaccid paralysis. These toxins are widely distributed in nature and relatively simple to produce in quantity. For these reasons, BoNTs are listed by the Centers of Disease Control and Prevention (CDC) as Tier 1 Select Agents. BoNTs also are highly variable in nature, with at least seven major serotypes (A-G) reported, most of which forms a group with multiple variant subtypes. This extreme natural variability seriously complicates the development of botulism treatments. Currently the only available therapeutics for botulism are equine antitoxin serum products which are administered intravenously to patients exposed to toxin. A goal in the field is to develop other antitoxins to replace equine antiserum products. However, while antitoxin treatments prevent further intoxication, they do not promote recovery from paralysis that has already occurred.

All BoNTs have a catalytic light chain (LC), which is a Zn²⁺-endopeptidase that specifically cleaves neuronal SNARE proteins and is mainly responsible for the neurotoxic effects of BoNTs, while the heavy chain (HC) mediates toxin attachment to neurons and delivers the LC into the cytosol. Needed in the art are biomolecular products that serve as treatments to rescue symptomatic botulism by inhibiting the LC activity of the intoxicating BoNTs, such as the LCs of BoNT serotypes A and B. The protein products and methods described herein address the needs in the art by providing effective and potent botulism antidotes that inhibit the LCs of BoNT and that are useful as inhibitory agents against a number of natural subtypes of each BoNT serotype.

### SUMMARY

In an aspect, a VHH polypeptide that specifically binds to a light chain (LC) protease domain of Botulinum neurotoxin (BoNT) serotype A (LC/A) or a LC/A-binding portion thereof, or to a light chain (LC) protease domain of Botulinum neurotoxin (BoNT) serotype B (LC/B), or a LC/B-binding portion thereof, is provided, in which the VHH polypeptide that binds to the LC/A neurotoxin comprises complementarity determining regions (CDRs), CDR1, CDR2 and CDR3, comprising amino acid sequences selected from:
CDR1: SGFTLDDYAIGWF (SEQ ID NO: 38); CDR2: SRSGDTYYP (SEQ ID NO: 39); and CDR3: DFPPVRPMCIQAAPKKR (SEQ ID NO: 40);
CDR1: SGADFSFYAMGWY (SEQ ID NO: 41); CDR2: NLNGVISYG (SEQ ID NO: 42); and CDR3: MRLYTRGSVRHPESW (SEQ ID NO: 43);
CDR1: TGRTLDYYALGWF (SEQ ID NO: 44); CDR2: NWLGGSTYYA (SEQ ID NO: 45); and CDR3: DFSIAYSGTYPPAYAEYDYDYW (SEQ ID NO: 46);
CDR1: SGSPLSIWVMGWY (SEQ ID NO: 47); CDR2: NLNGITSYG (SEQ ID NO: 48); and CDR3: EPLGPRGKKSGKEYW (SEQ ID NO: 49);
CDR1: TSENVFGIYGMAW (SEQ ID NO: 50); CDR2: ITSRGTAHYH (SEQ ID NO: 51); and CDR3: GPYW (SEQ ID NO: 52);
CDR1: SGFTFNRYVIRWY (SEQ ID NO: 53); CDR2: SRSGDSGRYV (SEQ ID NO: 54); and CDR3: LNLEDMEYW (SEQ ID NO: 55);
CDR1: SGNIFSIYYMGWY (SEQ ID NO: 56); CDR2: NSNGITNYG (SEQ ID NO: 57); and CDR3: GKLRRTTGWGLDDYW (SEQ ID NO: 58);
CDR1: SGFTLDEYAIGWF (SEQ ID NO: 59); CDR2: SSSASISYA (SEQ ID NO: 60); and CDR3: AFLACGPVAGWGTEYDYW (SEQ ID NO: 61);
CDR1: STTISDFYSMGWF (SEQ ID NO: 62); CDR2: RRGGDTKSG (SEQ ID NO: 63); and CDR3: NLQKSSDELGPYYW (SEQ ID NO: 64);
CDR1: SLLTLEYYAIGWF (SEQ ID NO: 65); CDR2: GSSGGSTVYI (SEQ ID NO: 66); and CDR3: DDLRCGRGWSSYFRGSW (SEQ ID NO: 67);
CDR1: SESVFEMYTVAWY (SEQ ID NO: 68); CDR2: TDEGRTNYA (SEQ ID NO: 69); and CDR3: EHDLGYYDYW (SEQ ID NO: 70);
CDR1: SGLTLDYYAIGWF (SEQ ID NO: 71); CDR2: SSGSSMSIHA (SEQ ID NO: 72); and CDR3: DDFTCGSRWSDWAHTFGFW (SEQ ID NO: 73); or
CDR1: SGGIFSTYIMGWY (SEQ ID NO: 74); CDR2: SNHTTDYA (SEQ ID NO: 75); and CDR3: DWMVGAWTAGDYGVDYW (SEQ ID NO: 76); and wherein the VHH polypeptide that binds to the LC/B neurotoxin comprises complementarity determining regions (CDRs), CDR1, CDR2 and CDR3, comprising amino acid sequences selected from:
CDR1: SGLSFNWYDVG (SEQ ID NO: 77); CDR2: SRSSGGGSTY (SEQ ID NO: 78); and CDR3: AADWTGRAGFSVGYYRPDEYDY (SEQ ID NO: 79);
CDR1: SGFTLDSYAIG (SEQ ID NO: 80); CDR2: CMSSGDGSTY (SEQ ID NO: 81); and CDR3: AADGFDYCSAYVPGRGMNY (SEQ ID NO: 82);
CDR1: SGFTLDNYAVG (SEQ ID NO: 83); CDR2: CISSSDDNTD (SEQ ID NO: 84); and CDR3: AAESPTFGFSCTVATDPYDY (SEQ ID NO: 85);
CDR1: SGFTLDGYAAG (SEQ ID NO: 86); CDR2: WISSTDGSTY (SEQ ID NO: 87); and CDR3: TAGLGLDVSDYVYDY (SEQ ID NO: 88);
CDR1: SGFTLDYYGIG (SEQ ID NO: 89); CDR2: CITSGGLTN (SEQ ID NO: 90); and CDR3: AIDRVGVCAMEDFGS (SEQ ID NO: 91);
CDR1: SGRTFNYYAMA (SEQ ID NO: 92); CDR2: FINWSGDSTY (SEQ ID NO: 93); and CDR3: AAEFGTFSYLQGDDYSY (SEQ ID NO: 94);
CDR1: SGRSFSSYRMG (SEQ ID NO: 95); CDR2: GISWSGSSTW (SEQ ID NO: 96); and CDR3: AADGLGTDWSDAIWDY (SEQ ID NO: 97);
CDR1: SGRMFNEYRMG (SEQ ID NO: 98); CDR2: AINWGAQIPY (SEQ ID NO: 99); and CDR3: AADWGYGSSPHQDKEYDY (SEQ ID NO: 100);
CDR1: SGRTFSDYAMG (SEQ ID NO: 101); CDR2: AVDWSGSSRL (SEQ ID NO: 102); and CDR3: AAARNRWSSEISSYDY (SEQ ID NO: 103);
CDR1: SGRTFRRNTMG (SEQ ID NO: 104); CDR2: AISWSGDRTY (SEQ ID NO: 105); and CDR3: AADGTASVFNSYASADRNKYNY (SEQ ID NO: 106);
CDR1: SIRTFSTSTTA (SEQ ID NO: 107); CDR2: RISGSDPVTY (SEQ ID NO: 108); and CDR3: ATVRIKGGSEFSYHY (SEQ ID NO: 109);
CDR1: SESTFSINAIG (SEQ ID NO: 110); CDR2: HISTSGRTR (SEQ ID NO: 111); and CDR3: NAEGYSTWPEDRYLEL (SEQ ID NO: 112);
CDR1: SGRTFSSYRMGMG (SEQ ID NO: 113); CDR2: TVNWSGGTTY (SEQ ID NO: 114); and CDR3: AAGRGSESYTSSRYNY (SEQ ID NO: 115); or
CDR1: SGFTLDDYAIG (SEQ ID NO: 116); CDR2: CISIRDGRTH (SEQ ID NO: 117); and CDR3: AAGQRSMAYVCSNRFGS (SEQ ID NO: 118). In an embodiment, the polypeptide of claim 1, further comprises a framework region. In an embodiment, the VHH polypeptide that binds to the LC/A neurotoxin comprises complementarity determining regions (CDRs), CDR1, CDR2 and CDR3, comprising amino acid sequences:
CDR1: SGADFSFYAMGWY (SEQ ID NO: 41); CDR2: NLNGVISYG (SEQ ID NO: 42); and CDR3: MRLYTRGSVRHPESW (SEQ ID NO: 43);
CDR1: TGRTLDYYALGWF (SEQ ID NO: 44); CDR2: NWLGGSTYYA (SEQ ID NO: 45); and CDR3: DFSIAYSGTYPPAYAEYDYDYW (SEQ ID NO: 46);
CDR1: SGFTFNRYVIRWY (SEQ ID NO: 53); CDR2: SRSGDSGRYV (SEQ ID NO: 54); and CDR3: LNLEDMEYW (SEQ ID NO: 55);
CDR1: SGNIFSIYYMGWY (SEQ ID NO: 56); CDR2: NSNGITNYG (SEQ ID NO: 57); and CDR3: GKLRRTTGWGLDDYW (SEQ ID NO: 58);
CDR1: SGFTLDEYAIGWF (SEQ ID NO: 59); CDR2: SSSASISYA (SEQ ID NO: 60); and CDR3: AFLACGPVAGWGTEYDYW (SEQ ID NO: 61); or
CDR1: STTISDFYSMGWF (SEQ ID NO: 62); CDR2: RRGGDTKSG (SEQ ID NO: 63); and CDR3: NLQKSSDELGPYYW (SEQ ID NO: 64); or wherein the VHH polypeptide that binds to the LC/B neurotoxin comprises complementarity determining regions (CDRs), CDR1, CDR2 and CDR3, comprising amino acid sequences:

CDR1:
(SEQ ID NO: 104)
SGRTFRRNTMG;
CDR2:
(SEQ ID NO: 105)
AISWSGDRTY;
and
CDR3:
(SEQ ID NO: 106)
AADGTASVENSYASADRNKYNY.

In an aspect, a VHH polypeptide that specifically binds to a light chain (LC) protease domain of Botulinum neurotoxin (BoNT) serotype A (LC/A) or a LC/A-binding portion thereof, or to a light chain (LC) protease domain of Botulinum neurotoxin (BoNT) serotype B (LC/B) or a LC/B-binding portion thereof, is provided, in which the VHH polypeptide that binds to the LC/A neurotoxin comprises an amino acid sequence having at least 85% sequence identity to an amino acid sequence of a LC/A toxin binding polypeptide selected from the group consisting of JPU-A1 (SEQ ID NO: 7); JPU-A5 (SEQ ID NO: 8); JPU-A11 (SEQ ID NO: 9); JPU-B5 (SEQ ID NO: 10); JPU-B9 (SEQ ID NO: 11); JPU-C1 (SEQ ID NO: 12); JPU-C10 (SEQ ID NO: 13); JPU-D12 (SEQ ID NO: 14); JPU-G3 (SEQ ID NO: 15); JPU-G7 (SEQ ID NO: 16); JPU-G11 (SEQ ID NO: 17); JPU-G12 (SEQ ID NO: 18); and JPU-H7 (SEQ ID NO: 19); or wherein the VHH polypeptide that binds to the LC/B neurotoxin comprises an amino acid sequence having at least 85% or at least 90% sequence identity to an amino acid sequence of a LC/B toxin binding polypeptide selected from the group consisting of JND-A12 (SEQ ID NO: 24); JND-B4 (SEQ ID NO: 25); JND-C7 (SEQ ID NO: 26); JND-E4 (SEQ ID NO: 27); JND-E5 (SEQ ID NO: 28); JND-E9 (SEQ ID NO: 29); JND-F3 (SEQ ID NO: 30); JSG-B8 (SEQ ID NO: 31); JSG-B10 (SEQ ID NO: 32); JSG-C1 (SEQ ID NO: 33); JSG-F6 (SEQ ID NO: 34); JSG-G1 (SEQ ID NO: 35); JSG-G10 (SEQ ID NO: 36); and JSG-G11 (SEQ ID NO: 37). In an embodiment, the VHH polypeptide that binds to LC/A comprises an amino acid sequence having at least 85% sequence identity to an amino acid sequence of a LC/A-binding polypeptide selected from the group consisting of JPU-A5 (SEQ ID NO: 8), JPU-A11 (SEQ ID NO: 9), JPU-C1 (SEQ ID NO: 12), JPU-C10 (SEQ ID NO: 13), JPU-D12 (SEQ ID NO: 14), or JPU-G3 (SEQ ID NO: 15); or wherein VHH polypeptide that binds to LC/B comprises an amino acid sequence having at least 85% sequence identity to the amino acid sequence of LC/B-binding polypeptide JSG-C1 (SEQ ID NO: 33). In embodiments of the foregoing, the VHH polypeptide that binds to the LC/A or LC/B toxins comprises an amino acid sequence having at least 88%, at least 90%, at least 93%, at least 95%, at least 96%, at least 97%, at least 98%, at least 99% or greater, sequence identity to the LC/A or LC/B binding VHH polypeptides of the above-delineated aspects.

In another aspect, a VHH polypeptide that specifically binds to a light chain (LC) protease domain of Botulinum neurotoxin (BoNT) serotype A (LC/A) or a LC/A-binding portion thereof, or to a light chain (LC) protease domain of Botulinum neurotoxin (BoNT) serotype B (LC/B) or a LC/B-binding portion thereof, is provided, in which the VHH polypeptide that binds to the LC/A neurotoxin comprises or consists of JPU-A1 (SEQ ID NO: 7); JPU-A5 (SEQ ID NO: 8); JPU-A11 (SEQ ID NO: 9); JPU-B5 (SEQ ID NO: 10); JPU-B9 (SEQ ID NO: 11); JPU-C1 (SEQ ID NO: 12); JPU-C10 (SEQ ID NO: 13); JPU-D12 (SEQ ID NO: 14); JPU-G3 (SEQ ID NO: 15); JPU-G7 (SEQ ID NO: 16); JPU-G11 (SEQ ID NO: 17); JPU-G12 (SEQ ID NO: 18); and JPU-H7 (SEQ ID NO: 19); or wherein the VHH polypeptide that binds to the LC/B neurotoxin comprises or consists of JND-A12 (SEQ ID NO: 24); JND-B4 (SEQ ID NO: 25); JND-C7 (SEQ ID NO: 26); JND-E4 (SEQ ID NO: 27); JND-E5 (SEQ ID NO: 28); JND-E9 (SEQ ID NO: 29); JND-F3 (SEQ ID NO: 30); JSG-B8 (SEQ ID NO: 31); JSG-B10 (SEQ ID NO: 32); JSG-C1 (SEQ ID NO: 33); JSG-F6 (SEQ ID NO: 34); JSG-G1 (SEQ ID NO: 35); JSG-G10 (SEQ ID NO: 36); and JSG-G11 (SEQ ID NO: 37). In an embodiment, associated with the VHH polypeptide, the LC/A neurotoxin-binding VHH polypeptide comprises or consists of JPU-A5 (SEQ ID NO: 8) or JPU-C10 (SEQ ID NO: 13); or wherein the LC/B neurotoxin-binding VHH polypeptide comprises or consists of JSG-C1 (SEQ ID NO: 33).

In an embodiment of the above-delineated aspects related to the VHH polypeptides and/or embodiments thereof, the polypeptide inhibits and/or neutralizes BoNT/A LC/A protease function or activity or inhibits and/or neutralizes BoNT/B LC/B protease function or activity. In an embodiment, of the polypeptide, conservative amino acid substitutions in the polypeptide comprise the at least 90% amino acid sequence identity. In an embodiment, the VHH polypeptide is a camelid-derived single domain anti-LC/A toxin or LC/B toxin VHH antibody. In an embodiment, the VHH polypeptide comprises one or more epitope tag sequences specifically bindable by an anti-epitope tag antibody or binding portion thereof. In an embodiment, the one or more epitope tag sequences comprises at least one of DELGPRLMGK (SEQ ID NO: 119) or GAPVPYPDPLEPR (SEQ ID NO: 120).

In an embodiment related to the above-delineated VHH polypeptides and/or the embodiments thereof, the polypeptide is in the form of a dimer or multimer. In an embodiment, the polypeptide comprises two or more anti-LC/A toxin and/or anti-LC/B toxin VHH polypeptides, or LC/A-toxin or LC/B-toxin binding portions thereof, joined with one or more linker peptides. In an embodiment, the one or more linker peptides is selected from GGGGS (SEQ ID NO: 121); GGGGSGGGGSGGGGS (SEQ ID NO: 122), or a functional portion thereof; EPKTPKPQGGGGSGGGGSGGGGSQGVQSQVQLVE (SEQ ID NO: 123); EPKTPKPQ (SEQ ID NO: 124); or a combination thereof. In another embodiment, the VHH polypeptide is dimeric and comprises (i) two anti-LC/A-toxin VHH polypeptides, same or different; (ii) two anti-LC/B-toxin VHH polypeptides, same or different; or (iii) a combination of an anti-LC/A-toxin VHH polypeptide and an anti-LC/B-toxin VHH polypeptide. In another embodiment, the polypeptide is multimeric and comprises (i) at least three anti-LC/A toxin VHH polypeptides, same or different; (ii) at least three anti-LC/B toxin VHH polypeptides, same or different; or (iii) a combination of at least three anti-LC/A-toxin VHH polypeptides and anti-LC/B-toxin VHH polypeptides.

In an aspect, an isolated polynucleotide encoding the VHH polypeptide of any of the above-delineated aspects and/or embodiments thereof is provided.

In an aspect, a vector comprising the isolated polynucleotide encoding the VHH polypeptide of any of the above-delineated aspects and/or embodiments thereof is provided. In an embodiment, the vector is a viral or a non-viral expression vector.

In an aspect, a host cell comprising the above-described vector is provided.

In an aspect, a pharmaceutical composition comprising an effective amount of the VHH polypeptide of any of the above-delineated aspects and/or embodiments thereof and a pharmaceutically acceptable excipient, carrier, or diluent, is provided.

In an aspect, a method of treating botulism or intoxication associated with activity of Botulinum LC/A and/or LC/B neurotoxins, and/or the symptoms thereof, is provided, in which the method involves administering to a subject in need thereof an effective amount of the VHH polypeptide of any of the above-delineated aspects and/or embodiments thereof, or a polynucleotide encoding the polypeptide, or a pharmaceutical composition thereof, thereby treating botulism or infection by Botulinum microorganisms of serotype A and/or B, and/or the symptoms thereof in the subject.

In an aspect, a method of reducing the severity of botulism or intoxication associated with activity of Botulinum LC/A and/or LC/B neurotoxins, and/or the symptoms thereof, in a subject who has, who is susceptible to, or who is at risk of having botulism or intoxication by Botulinum microorganisms, is provided, in which the method involves administering to a subject in need thereof an effective amount of the VHH polypeptide of any of the above-delineated aspects and/or embodiments thereof, or a polynucleotide encoding the polypeptide, or a pharmaceutical composition thereof, thereby reducing the severity of botulism or intoxication by the LC/A and/or LC/B neurotoxin-producing Botulinum microorganisms and/or the symptoms thereof in the subject.

In an embodiment of the above methods, the method involves administering to the subject an anti-epitope tag antibody that specifically binds to an epitope tag, if present, and facilitates clearance of a complex of LC/A and/or LC/B bound to the anti-LC/A and/or anti-LC/B VHH polypeptide from the subject. In an embodiment of the methods, the VHH polypeptide provides post-exposure or late post-exposure therapeutic treatment for patients suffering from botulism. In an embodiment, the VHH polypeptide provides post-exposure or late post-exposure therapeutic treatment for patients suffering from botulism.

In an aspect, a kit comprising the polypeptide of any of the above-delineated aspects and/or embodiments thereof, or a pharmaceutical composition thereof, is provided for treating or protecting against disease or intoxication and/or the symptoms thereof caused by Botulinum LC/A toxin protease and/or Botulinum LC/B toxin protease. In an embodiment, the kit includes instructions for use.

In an aspect, a method of detecting botulinum protease LC/A and/or LC/B in a sample is provide, in which the method involves contacting the sample with one or more VHH polypeptides of claim 1 under conditions for detecting the binding of the VHH polypeptide to the LC/A and/or the LC/B in the sample. In an embodiment, the sample is a biological sample obtained from a subject or from a natural or environmental source.

### Definitions

Unless defined otherwise, all technical and scientific terms used herein have the meaning commonly understood by a person skilled in the art to which the described aspects and embodiments belongs. The following references provide one of skill with a general definition of many of the terms used in the aspects and embodiments described herein: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). The following terms have the meanings ascribed to them below, unless specified otherwise.

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in the specification and claim(s) herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions and products of the present disclosure can be used to achieve methods of the present disclosure.

Unless specifically stated or obvious from context, as used herein, the term “about” or “approximately” means within an acceptable error range for the particular value as determined by one of ordinary skill in the art, which will depend, in part, on how the value is measured or determined, i.e., the limitations of the measurement system. For example, “about” can mean within 1 standard deviation or more than 1 standard deviation, e.g., 2 standard deviations of the mean, as typically practiced in the art. Alternatively, and without intending to be limiting, “about” can mean a range of up to 20%, up to 10%, up to 5%, up to 2%, or up to 1% of a given value. Alternatively, and particularly for biological systems or processes, the term can mean within an order of magnitude, e.g., within 5-fold, within 3-fold, within 2.5-fold, or within 2-fold of a value. Where particular values are described in the application and claims, unless otherwise stated, the term “about” means within an acceptable error range for the particular value.

Reference herein to “some embodiments,” “an embodiment,” “one embodiment” or “other embodiments” means that a particular feature, structure, or characteristic described in connection with the embodiments is included in at least some embodiments, but not necessarily all embodiments, of the disclosure.

By “agent” is meant any small molecule chemical compound, antibody, nucleic acid molecule, or polypeptide (e.g., antibody or VHH antibody), or fragments thereof.

By “ameliorate” is meant decrease, reduce, diminish, suppress, attenuate, arrest, or stabilize the development or progression of a disease or pathology.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “antibody” is meant any immunoglobulin polypeptide, or fragment thereof, having immunogen or antigen binding ability. Antibody structure is well known in the art. Briefly, the variable (V) regions or domains of antibody heavy (H) and light (L) chains contain Complementarity-Determining Regions (CDRs), which bind to specific antigens or immunogens (e.g., protein antigens or immunogens). CDRs are situated within framework (FR) sequences of the V regions of the heavy (V_{H}) and light chains (V_{L}) of an antibody. CDRs are the most variable parts of antibodies and are critical components in the diversity of antigen specificities of antibodies produced by B lymphocytes. In general, three CDRs (CDR1, CDR2 and CDR3) are arranged consecutively in a V domain of an antibody. Because a VHH, such as a camelid VHH, is essentially a single chain antibody polypeptide, it contains three CDRs that bind to an antigen or target protein such as a toxin or neurotoxin (e.g., LC/A or LC/B BoNT) in the context of four framework (FR) regions, as follows: FR1-CDR1-FR2-CDR2-FR3-CDR3-FR4. Thus, as would be appreciated by the skilled practitioner in the art, in a VHH polypeptide sequence, FR1 comprises the amino acids positioned to the left of CDR1; FR2 comprises the amino acids positioned between CDR1 and CDR2; FR3 comprises the amino acids positioned between CDR2 and CDR3; and FR4 comprises the amino acids positioned to the right of CDR3. Because most of the sequence variability associated with immunoglobulins and antigen binding is found in the CDRs, these regions are sometimes referred to as hypervariable regions. Typically, CDR1, CDR2 and CDR3 of VHHs contribute to and/or do not interfere with antigen binding. The CDRs of a number of BoNT LC/A or LC/B VHHs described herein are shown, for example, the Figures and Supplementary Figures described and shown herein. The FRs of a number of BoNT LC/A or LC/B VHHs described herein are shown, for example, in the Figures and Supplementary Figures described and shown herein.

Botulinum neurotoxin Type A light chain protease (BoNT/LCA) refers to a polypeptide (protein) having at least 85%, at least 90%, at least 95%, at least 98%, or at least 99% sequence identity to the protein having UniProtKB/Swiss-Prot Reference No. P0DPI0.1 and having the amino acid sequence presented below:

| (SEQ ID NO: 125) | |
|---|---|
| 1 | MPFVNKQFNY KDPVNGVDIA YIKIPNVGQM QPVKAFKIHN KIWVIPERDT FTNPEEGDLN |
| 61 | PPPEAKQVPV SYYDSTYLST DNEKDNYLKG VTKLFERIYS TDLGRMLLTS IVRGIPFWGG |
| 121 | STIDTELKVI DTNCINVIQP DGSYRSEELN LVIIGPSADI IQFECKSFGH EVLNLTRNGY |
| 181 | GSTQYIRFSP DFTFGFEESL EVDTNPLLGA GKFATDPAVT LAHELIHAGH RLYGIAINPN |
| 241 | RVFKVNTNAY YEMSGLEVSF EELRTFGGHD AKFIDSLQEN EFRLYYYNKF KDIASTLNKA |
| 301 | KSIVGTTASL QYMKNVFKEK YLLSEDTSGK FSVDKLKFDK LYKMLTEIYT EDNFVKFFKV |
| 361 | LNRKTYLNFD KAVFKINIVP KVNYTIYDGF NLRNTNLAAN FNGQNTEINN MNFTKLKNFT |
| 421 | GLFEFYKLLC VRGIITSKTK SLDKGYNKAL NDLCIKVNNW DLFFSPSEDN FTNDLNKGEE |
| 481 | ITSDTNIEAA EENISLDLIQ QYYLTFNFDN EPENISIENL SSDIIGQLEL MPNIERFPNG |
| 541 | KKYELDKYTM FHYLRAQEFE HGKSRIALTN SVNEALLNPS RVYTFFSSDY VKKVNKATEA |
| 601 | AMFLGWVEQL VYDFTDETSE VSTTDKIADI TIIIPYIGPA LNIGNMLYKD DFVGALIFSG |
| 661 | AVILLEFIPE IAIPVLGTFA LVSYIANKVL TVQTIDNALS KRNEKWDEVY KYIVTNWLAK |
| 721 | VNTQIDLIRK KMKEALENQA EATKAIINYQ YNQYTEEEKN NINFNIDDLS SKLNESINKA |
| 781 | MININKFLNQ CSVSYLMNSM IPYGVKRLED FDASLKDALL KYIYDNRGTL IGQVDRLKDK |
| 841 | VNNTLSTDIP FQLSKYVDNQ RLLSTFTEYI KNIINTSILN LRYESNHLID LSRYASKINI |
| 901 | GSKVNFDPID KNQIQLFNLE SSKIEVILKN AIVYNSMYEN FSTSFWIRIP KYFNSISLNN |
| 961 | EYTIINCMEN NSGWKVSLNY GEIIWTLQDT QEIKQRVVFK YSQMINISDY INRWIFVTIT |
| 1021 | NNRLNNSKIY INGRLIDQKP ISNLGNIHAS NNIMFKLDGC RDTHRYIWIK YFNLFDKELN |
| 1081 | EKEIKDLYDN QSNSGILKDF WGDYLQYDKP YYMLNLYDPN KYVDVNNVGI RGYMYLKGPR |
| 1141 | GSVMTTNIYL NSSLYRGTKF IIKKYASGNK DNIVRNNDRV YINVVVKNKE YRLATNASQA |
| 1201 | GVEKILSALE IPDVGNLSQV VVMKSKNDQG ITNKCKMNLQ DNNGNDIGFI GFHQFNNIAK |
| 1261 | LVASNWYNRQ IERSSRTLGC SWEFIPVDDG WGERPL |

Botulinum neurotoxin Type B light chain protease (BoNT/LCB) refers to a polypeptide (protein) having at least 85%, at least 90%, at least 95%, at least 98%, or at least 99% sequence identity to the protein having UniProtKB/Swiss-Prot Reference No. P10844.3 and having the amino acid sequence presented below:

| (SEQ ID NO: 126) | |
|---|---|
| 1 | MPVTINNFNY NDPIDNNNII MMEPPFARGT GRYYKAFKIT DRIWIIPERY TFGYKPEDFN |
| 61 | KSSGIFNRDV CEYYDPDYLN TNDKKNIFLQ TMIKLFNRIK SKPLGEKLLE MIINGIPYLG |
| 121 | DRRVPLEEFN TNIASVTVNK LISNPGEVER KKGIFANLII FGPGPVLNEN ETIDIGIQNH |
| 181 | FASREGFGGI MQMKFCPEYV SVFNNVQENK GASIFNRRGY FSDPALILMH ELIHVLHGLY |
| 241 | GIKVDDLPIV PNEKKFFMQS TDAIQAEELY TFGGQDPSII TPSTDKSIYD KVLQNFRGIV |
| 301 | DRINKVLVCI SDPNININIY KNKFKDKYKF VEDSEGKYSI DVESFDKLYK SLMFGFTETN |
| 361 | IAENYKIKTR ASYFSDSLPP VKIKNLLDNE IYTIEEGFNI SDKDMEKEYR GQNKAINKQA |
| 421 | YEEISKEHLA VYKIQMCKSV KAPGICIDVD NEDLFFIADK NSFSDDLSKN ERIEYNTQSN |
| 481 | YIENDFPINE LILDTDLISK IELPSENTES LTDFNVDVPV YEKQPAIKKI FTDENTIFQY |
| 541 | LYSQTFPLDI RDISLTSSFD DALLFSNKVY SFFSMDYIKT ANKVVEAGLF AGWVKQIVND |
| 601 | FVIEANKSNT MDKIADISLI VPYIGLALNV GNETAKGNFE NAFEIAGASI LLEFIPELLI |
| 661 | PVVGAFLLES YIDNKNKIIK TIDNALTKRN EKWSDMYGLI VAQWLSTVNT QFYTIKEGMY |
| 721 | KALNYQAQAL EEIIKYRYNI YSEKEKSNIN IDFNDINSKL NEGINQAIDN INNFINGCSV |
| 781 | SYLMKKMIPL AVEKLLDFDN TLKKNLLNYI DENKLYLIGS AEYEKSKVNK YLKTIMPFDL |
| 841 | SIYTNDTILI EMFNKYNSEI LNNIILNLRY KDNNLIDLSG YGAKVEVYDG VELNDKNQFK |
| 901 | LTSSANSKIR VTQNQNIIFN SVFLDFSVSF WIRIPKYKND GIQNYIHNEY TIINCMKNNS |
| 961 | GWKISIRGNR IIWTLIDING KTKSVFFEYN IREDISEYIN RWFFVTITNN LNNAKIYING |
| 1021 | KLESNTDIKD IREVIANGEI IFKLDGDIDR TQFIWMKYFS IFNTELSQSN IEERYKIQSY |
| 1081 | SEYLKDFWGN PLMYNKEYYM FNAGNKNSYI KLKKDSPVGE ILTRSKYNQN SKYINYRDLY |
| 1141 | IGEKFIIRRK SNSQSINDDI VRKEDYIYLD FFNLNQEWRV YTYKYFKKEE EKLFLAPISD |
| 1201 | SDEFYNTIQI KEYDEQPTYS CQLLFKKDEE STDEIGLIGI HRFYESGIVF EEYKDYFCIS |
| 1261 | KWYLKEVKRK PYNLKLGCNW QFIPKDEGWT E |

A “chimeric antibody” refers to an antibody in which the constant region of an antibody of one species (e.g., rodent, mouse or rat) is replaced with that from a human to achieve a more human-like antibody. Chimeric antibodies may be recombinantly generated by combining the variable light and heavy chain regions obtained from antibody producing cells of one species with the constant light and heavy chain regions from another. In general, chimeric antibodies utilize rodent (or other species, such as rabbit or camelid) variable regions and human constant regions in order to produce an antibody with predominantly human constant domains. The production of chimeric antibodies is well known in the art, and may be achieved by standard means, for example, as described in U.S. Pat. No. 5,624,659, incorporated fully herein by reference.

By “binding to” a molecule is meant having a physicochemical affinity for that molecule or a region of the molecule, e.g., an epitope. Binding may be measured by any of the methods practiced in the art, e.g., using an antibody binding assay or an in vitro translation binding assay. “Detect” refers to identifying or determining the presence, absence or amount of an analyte to be detected.

By “detectable label” is meant a compound, substance, or composition that, when linked to a molecule of interest, renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition, disorder, or pathology that damages or interferes with the normal function of a cell, tissue, or organ. Examples of diseases include, without limitation, botulism caused by Botulinum serotype A and/or B neurotoxins, pathologies and conditions associated with the production of Botulinum neurotoxins, i.e., intoxication by Botulinum neurotoxin LC/A and/or LC/B, and/or one or more symptoms of these diseases, pathologies, and conditions following infection by Botulinum microorganisms. The production of toxins by the pathogenic and infectious Botulinum microorganisms results in intoxication of the subject (or patient), which is an abnormal state that constitutes a poisoning of the subject (and the subject's cells, tissues and organs) associated with the presence and activity of the produced toxins.

By “effective amount” is meant the amount of a required to ameliorate, or optimally eliminate, the symptoms of a disease relative to an untreated patient. The effective amount of active compound(s) used to practice the present aspects and embodiments for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

An “epitope tag” refers to a peptide or amino acid sequence (epitope) that is fused, linked, or coupled to a protein, such as a recombinant protein produced by recombinant techniques, and that can be specifically bound by an antibody, e.g., an anti-tag monoclonal antibody or binding molecule that is directed to or generated against the tag peptide or amino acid sequence. Epitope tags are typically short peptide sequences (e.g., from about 5-30 amino acids, or sometimes up to 40 amino acids, that are selected because high-affinity antibodies can be reliably produced in many different species. Such anti-epitope tag antibodies are optimally not cross-reactive with other human peptides or polypeptides and typically do not generate an antibody response, e.g., an anti-tag antibody response, when administered or delivered to a subject. An epitope tag sequence that is fused to a protein provides for the detection and/or purification of the protein using an antibody, e.g., a monoclonal antibody, that specifically binds to the epitope tag. In an embodiment, the protein to which an epitope tag is fused, linked, or coupled is an antibody or VHH protein, e.g., a recombinantly produced antibody or VHH protein. In an embodiment, the VHH is an anti-LC/A or anti-LC/B BoNT VHH antibody that binds BoNT LC/A or LC/B. In an embodiment, the protein, or a dimeric or multimeric form thereof, may include one or more epitope tags. In an embodiment, an epitope tag is coupled to the amino (NH) terminus of the protein, e.g., a VHH antibody as described herein. In an embodiment, an epitope tag is coupled to the carboxy (COOH) terminus of the protein, e.g., a VHH antibody as described herein. In an embodiment, an epitope tag is coupled to the NH and the COOH termini of the protein, e.g., a VHH antibody as described herein. In an embodiment, a dimeric or multimeric form of the protein includes one or more, e.g., two, three or four, epitope tags linked to one or more of the VHHs comprising the dimeric or multimeric form of the protein. Such epitope tags may be coupled to the VHH components at locations within the dimer or multimer molecule, or at the NH and/or COOH termini of the molecule. In some embodiments, two or more epitope tags may be coupled to a VHH protein in tandem within or at the termini of the VHH protein or dimeric or multimeric form thereof. Examples of epitope tags include, without limitation, FLAG tags (peptide sequence DYKDDDDK (SEQ ID NO: 127) recognized by an anti-FLAG antibody), polyHistidine (His) tags (5-10 histidine residues (SEQ ID NO: 128) (HHHHHH (SEQ ID NO: 129)) bound by a nickel or cobalt chelate), E-tag, a peptide comprising amino acid sequence GAPVPYPDPLEPR (SEQ ID NO: 120) recognized by an antibody; and the epitope tag sequences described herein, which are bound by anti-epitope tag antibodies, forming complexes which may facilitate clearance of the protein containing the tags from the body or system. (See, also, B. Brizzard and R. Chubet, 2001, Curr Protoc Neurosci., Chapter 5, Unit 5.8; DOI: 10.1002/0471142301.ns0508s00; R. Hernan et al., 2000, Biotechniques, 28(4):789-793; C. E. Fritze et al., 2000, Meths Enzymol., 327:3-16; doi: 10.1016/s0076-6879(00)27263-7; A. Einhauer et al., 2001, J Biochem Biophys Methods, 49(1-3):455-65, doi: 10.1016/s0165-022x(01)00213-5)).

A “framework (FR) region” or “FR region” includes amino acid residues that are adjacent to the CDRs in V_{H}, and V_{L} regions, and in VHHs. For example, FR region residues may be present in VHHs as described herein, human antibodies, rodent-derived antibodies (e.g., murine and rat antibodies), humanized antibodies, primatized antibodies, chimeric antibodies, antibody fragments (e.g., Fab fragments), VHHs, single-chain antibody fragments (e.g., scFv fragments), antibody domains, and bispecific antibodies, among others. Also by way of example, in a VHH polypeptide sequence, FR1 comprises the amino acids positioned to the left of CDR1; FR2 comprises the amino acids positioned between CDR1 and CDR2; FR3 comprises the amino acids positioned between CDR2 and CDR3; and FR4 comprises the amino acids positioned to the right of CDR3.

By “fragment” is meant a portion of a polypeptide or nucleic acid molecule. This portion contains, preferably, at least 10%, 20%, 30%, 40%, 50%, 60%, 70%, 80%, 90%, or 95% of the entire length of the reference nucleic acid molecule or polypeptide, including percent values between those enumerated. A fragment may contain 10, 20, 30, 40, 50, 60, 70, 80, 90, or 100, 200, 300, 400, 500, 600, 700, 800, 900, or 1000 nucleotides or amino acids. In an embodiment, a fragment or portion possesses or retains activity or function of the polypeptide from which it is derived. In embodiments, a fragment is derived from a BoNT LC/A toxin or a BoNT LC/B toxin.

The term “humanized” antibodies refers to forms of non-human (e.g., murine) antibodies, camelid-derived single domain antibody (sdAb) binding molecules, which are comprised of the heavy chain variable (V_{H}) region of heavy-chain-only antibodies (Abs) or VHHs. Humanized antibodies include chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, a humanized antibody or VHH may comprise substantially all of at least one variable domain (or two variable domains in the case of non-VHH antibodies), in which all or substantially all of the CDR regions correspond to those of a non-human immunoglobulin. All or substantially all of the FR regions of a humanized antibody may also be derived from a human immunoglobulin sequence. In the case of non-VHH antibodies, a humanized antibody can also comprise at least a portion of an immunoglobulin constant region (Fc), which may be that of a human immunoglobulin consensus sequence. Techniques and protocols for humanizing antibodies (as well as VHHs) are known and practiced in the art, as described, for examples, in Riechmann et al., Nature, 332:323-7, 1988; Kasmiri et al., Methods, 36(1):25-34, 2005; U.S. Pat. Nos. 5,530,101; 5,585,089; 5,693,761; 5,693,762; and U.S. Pat. No. 6,180,370 to Queen et al; EP239400; WO 1991/09967; U.S. Pat. No. 5,225,539; EP592106; and EP519596, the contents of which are incorporated herein by reference. Humanized antibodies or VHHs are molecularly engineered to contain even more human-like immunoglobulin domains, and incorporate only the CDRs of the VHH or animal-derived monoclonal antibody by carefully examining the sequence of the hyper-variable loops of the V regions of the monoclonal antibody or VHH, and fitting them to the structure of the human antibody chains. This process is routinely and commonly carried out by one having skill in the art. See, e.g., U.S. Pat. No. 6,187,287, the contents of which are incorporated by reference herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of the aspects and embodiments disclosed and described herein is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the terms “polynucleotide,” “DNA molecule” or “nucleic acid molecule” include both sense and anti-sense strands, cDNA, genomic DNA, recombinant DNA, RNA, mRNA, and wholly or partially synthesized nucleic acid molecules. A nucleotide “variant” is a sequence that differs from the recited nucleotide sequence in having one or more nucleotide deletions, substitutions or additions. Such modifications are readily introduced using standard mutagenesis techniques, such as oligonucleotide-directed site-specific mutagenesis as described, for example, in Adelman et al., 1983, DNA 2:183. Nucleotide variants are naturally-occurring allelic variants, or non-naturally occurring variants. Variant nucleotide sequences in various embodiments exhibit at least about 70%, 75%, 80%, 85%, 90%, 95%, 98%, 99%, or 100% sequence homology or sequence identity to the recited sequence. Such variant nucleotide sequences hybridize to the recited nucleotide sequence under stringent hybridization conditions. In one embodiment, “stringent conditions” refers to prewashing in a solution of 6×SSC, 0.2% SDS; hybridizing at 65° Celsius, 6×SSC, 0.2% SDS overnight; followed by two washes of 30 minutes each in 1×SSC, 0.1% SDS at 65° C., and two washes of 30 minutes each in 0.2×SSC, 0.1% SDS at 65° C.

By “isolated polynucleotide” is meant a nucleic acid (e.g., DNA, cDNA, RNA, mRNA) that is free of the genes, which, in the naturally-occurring genome of the organism from which the nucleic acid molecule of the aspects and embodiments described herein is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, e.g., mRNA, as well as a recombinant DNA that is part of a hybrid gene encoding additional polypeptide sequence.

The terms “protein”, “peptide” and “polypeptide” are used herein to describe any chain of amino acid residues, regardless of length or post-translational modification (for example, glycosylation or phosphorylation). Thus, these terms can be used interchangeably herein to refer to a polymer of amino acid residues. The terms also apply to amino acid polymers in which one or more amino acid residue is an artificial chemical mimetic of a corresponding naturally occurring amino acid. Thus, the term “polypeptide” includes full-length proteins, which may be, but need not be, naturally occurring, as well as recombinantly or synthetically produced polypeptides that correspond to a full-length protein, or to particular domains or portions of a protein, which may be, but need not be, naturally occurring. The term also encompasses mature proteins which have an added amino-terminal methionine to facilitate expression in prokaryotic cells. The binding molecules of the aspects and embodiments described herein are encoded by polynucleotides and can be chemically synthesized or synthesized by recombinant DNA methods.

By an “isolated polypeptide” is meant a polypeptide of the aspects and embodiments described herein that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the aspects and embodiments described herein. An isolated polypeptide of the aspects and embodiments described herein may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, “obtaining” as in “obtaining an agent” includes synthesizing, purchasing, deriving, isolating, or otherwise acquiring the agent.

By “operably linked” is meant the connection between regulatory elements and one or more polynucleotides (genes) or a coding region. That is, gene expression is typically placed under the control of certain regulatory elements, including constitutive or inducible promoters, tissue-specific regulatory elements, and enhancers. A polynucleotide (gene or genes) or coding region is said to be “operably linked to” or “operatively linked to” or “operably associated with” the regulatory elements, meaning that the polynucleotide (gene or genes) or coding region is controlled or influenced by the regulatory elements. The one or more polynucleotides may be separated by spacers or linkers.

By “pathogen” is meant any harmful microorganism, bacterium, virus, fungus, or protozoan capable of interfering with the normal function of a cell. Pathogens as referred to herein produce toxins (also referred to as neurotoxins herein), e.g., protein toxins, that intoxicate the cells, tissues and organs of a host or recipient organism and cause disease and pathology, often severe, unless they are bound by, neutralized and eliminated from the organism to the extent possible, such as by action of the VHH binding molecules (antibodies) described herein. As described herein, the bacterial Botulinum pathogen produces neurotoxin proteins that intoxicate a subject after infection. The catalytic light chain (LC) of the toxin is a Zn²-endopeptidase that specifically cleaves neuronal SNARE proteins and is mainly responsible for BoNT's neurotoxic effects, while the heavy chain (HC) mediates the attachment of toxin to neurons and delivers the LC into the cytosol. In particular, the VHH antibodies described herein are directed against and bind the BoNT LC/A or LC/B. In embodiments, the anti-LC/A or anti-LC/B VHHs described herein inhibit, block, or reduce neurotoxicity and toxic effects in vitro or in vivo.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative or lowering alteration of at least 5%, 10%, 15%, 10%, 25%, 50%, 75%, or 100%.

By “reference” is meant a standard or control condition typically used as a comparator in an assay, test, experiment, or trial, as would be understood by one having skill in the pertinent art. In various nonlimiting embodiments, a reference or control is a different or nonpathogenic protein or cell, such as a non-toxin (or different toxin) protein or a normal cell, a wild-type (unmutated or unaltered) protein, or a healthy subject or individual.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound, molecule, antibody, or VHH that recognizes and binds a protein, peptide, or polypeptide (e.g., an amino acid sequence of the protein, peptide, or polypeptide), but which does not substantially recognize and bind other molecules in a sample, for example, a biological sample, which may contain the protein, peptide, or polypeptide that is specifically bound.

“Nucleic acid” (also called polynucleotide herein) refers to deoxyribonucleotides or ribonucleotides and polymers thereof in either single- or double-stranded form. The term encompasses nucleic acids (polynucleotides) containing known nucleotide analogs or modified backbone residues or linkages, which are synthetic, naturally occurring, and non-naturally occurring, which have similar binding properties as a reference nucleic acid, and which are metabolized in a manner similar to the reference nucleic acid. Examples of such analogs include, without limitation, phosphorothioates, phosphoramidates, methyl phosphonates, chiral methyl phosphonates, 2-O-methyl ribonucleotides, and peptide-nucleic acids (PNAs). Unless otherwise indicated, a particular nucleic acid sequence also implicitly encompasses conservatively modified variants thereof (for example, degenerate codon substitutions) and complementary sequences, as well as the sequence explicitly indicated. Specifically, degenerate codon substitutions may be achieved by generating sequences in which the third position of one or more selected (or all) codons is substituted with suitable mixed base and/or deoxyinosine residues (Batzer et al., 1991, Nucleic Acid Res, 19:081; Ohtsuka et al., 1985, J. Biol. Chem., 260:2600-2608; Rossolini et al., 1994, Mol. Cell Probes, 8:91-98). The term nucleic acid can be used interchangeably with gene, cDNA, mRNA, oligonucleotide, and polynucleotide. Nucleic acid molecules or polynucleotides useful in the aspects and embodiments described herein include any nucleic acid molecule or polynucleotide that encodes a polypeptide, e.g., a heteromultimeric binding molecule, of the described aspects and embodiments, or a component or portion thereof. Nucleic acid molecules useful in the methods described herein include any polynucleotide or nucleic acid molecule that encodes a polypeptide e.g., heteromultimeric binding molecule, as described in the aspects and embodiments herein, or a component or portion thereof that has substantial identity to the binding molecule. Such nucleic acid molecules need not be 100% identical with the nucleic acid sequence of the binding molecule, but will typically exhibit substantial identity. Polynucleotides having “substantial identity” to a binding molecule sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger, 1987, Methods Enzymol. 152:399; Kimmel, A. R., 1987, Methods Enzymol. 152:507). For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an embodiment, hybridization will occur at 30° C. in 750 mM NaCl, 75 mM trisodium citrate, and 1% SDS. In an embodiment, hybridization will occur at 37° C. in 500 mM NaCl, 50 mM trisodium citrate, 1% SDS, 35% formamide, and 100 μg/ml denatured salmon sperm DNA (ssDNA). In an embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In an embodiment, wash steps will occur at 25° C. in 30 mM NaCl, 3 mM trisodium citrate, and 0.1% SDS. In an embodiment, wash steps will occur at 42° C. in 15 mM NaCl, 1.5 mM trisodium citrate, and 0.1% SDS. In an embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Percentage of sequence identity” means the value determined by comparing two optimally aligned sequences over a comparison window, wherein the portion of the polynucleotide sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “homologous” in their various grammatical forms in the context of polynucleotides means that a polynucleotide comprises a sequence that has a desired identity, for example, at least 60% identity, at least 70% sequence identity, at least 80%, at least 85% identity, at least 90% identity; and at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like.

Substantial identity of amino acid sequences, for example, the anti-LC/A and anti-LC/B polypeptides (BoNT LC/A or LC/B-binding VHH polypeptides) refers to sequence identity between or among amino acid sequences of at least 80%, at least 85%, at least 88%, at least 90%, at least 93%, at least 95%, at least 96%, at least 97%, at least 98%, at least 99% or greater, sequence identity, as well as percentages therebetween. In embodiments, 100% identity between or among the amino acid sequences of the anti-LC/A or LC/B binding VHHs as described herein is not required for binding of these polypeptides to the LC/A or LC/B toxins and/or neutralization of toxin activity or inhibition of toxin activity. In a particular embodiment, variations between or among VHH amino acid sequences encompass one or more conservative amino acid substitutions in the sequence. In an embodiment, one or more conservative amino acid substitutions in an anti-LC/A or LC/B binding VHH polypeptide amino acid sequence may be in one or more CDR sequences, one or more FR sequences, or a combination thereof.

As will be appreciated by the skilled practitioner in the art, some amino acids in a VHH antibody can be modified without significantly altering antigen binding of the VHH antibody. For example, such amino acid sequence modification occurs frequently during in vivo affinity maturation of VHH antibodies, and the best mutations, e.g., for specific and/or high affinity binding to antigen, are positively selected for in the animal during the molecular production of antibodies. It is possible to isolate different VHH intermediates in the affinity maturation process that possess acceptable and specific antigen binding properties and that have significant variations in their CDR sequences. In an embodiment, an anti-LC/A toxin VHH polypeptide or an anti-LC/B toxin VHH polypeptide comprises the CDRs of the respective VHH polypeptides set forth in Tables 1 and 2 herein and comprise framework sequences having at least 88%, at least 90%, at least 93%, at least 95%, at least 96%, at least 97%, at least 98%, at least 99% or greater, sequence identity to the sequences of the VHH polypeptides depicted in FIG. 8 or FIG. 9, as well as percentages therebetween. In other embodiments, an anti-LC/A toxin VHH polypeptide or an anti-LC/B toxin VHH polypeptide comprises an amino acid sequence having at least 88%, at least 90%, at least 93%, at least 95%, at least 96%, at least 97%, at least 98%, at least 99% or greater, sequence identity (including percentages therebetween), identity to the sequences of the VHH polypeptides depicted in FIG. 8 or FIG. 9, e.g., LC/A toxin binding VHH polypeptides of SEQ ID NOS: 7-19, or LC/B toxin binding VHH polypeptides of SEQ ID NOS: 24-37.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as, without limitation, a human or a non-human primate, or a bovine, equine, canine, ovine, or feline mammal. Other mammals include rabbits, goats, llamas, mice, rats, guinea pigs, camels and gerbils. In particular, a “subject” as used herein refers to a human subject, such as a human patient or individual, particularly a patient who is suffering from botulism, Botulinum intoxication, and/or the symptoms thereof. In some cases, the terms subject, patient and individual are used interchangeably herein.

A “VHH binding molecule” or “VHH antibody,” or simply “VHH,” as referred to herein is, in general, a single domain immunoglobulin molecule (antibody) isolated from camelid animals or alpacas, e.g., as described in Maass, D. R., 2007, J. Immunol. Methods, 324(1-2):13-15). A VHH (or VHH antibody) corresponds to the heavy chain of a camelid antibody having a single variable domain (or single variable region), e.g., a camelid-derived single variable H (V_{H}) domain antibody. A VHH has a molecular weight (MW) of about 15 kDa. VHH technology is based on fully functional antibodies from camelids that lack light chains. These heavy-chain antibody molecules contain a single variable domain (VHH) and, typically, two constant domains (CH₂ and CH₃). A cloned (recombinantly produced) and isolated VHH domain is a stable polypeptide harboring the antigen-binding capacity of the original heavy-chain antibody. See, e.g., U.S. Pat. Nos. 5,840,526 and 6,015,695, each of which is incorporated by reference herein in its entirety. VHHs, called NANOBODIES™, may be produced commercially (Ablynx Inc., Ghent, Belgium).

VHHs are efficiently expressed in E. coli, coupled to detection markers, such as a fluorescent marker, or conjugated with enzymes. The small size of VHHs permits their binding to epitopes (antigenic determinants in antigen proteins), e.g., “hidden epitopes” that are not accessible to whole antibodies of much larger size. As a therapeutic, a VHH is capable of efficient penetration and rapid clearance. Its single domain nature allows a VHH to be expressed in a cell without a requirement for supramolecular assembly, as is needed for whole antibodies which are typically tetrameric (two heavy chains and two light chains, having a MW of about 150 kDa). VHHs are also exhibit stability over time and have a longer half-life versus non-VHH antibody molecules, which comprise disulfide bonds that are susceptible to chemical reduction or enzymatic cleavage. Similar to immunoglobulins, VHHs may be modified post-translationally, e.g. to add chemical linkers, detectable moieties, such as fluorescent dyes, enzymes, substrates, chemiluminescent moieties, etc., or specific binding moieties, such as streptavidin, avidin, or biotin, etc., for use in the compositions and methods described herein.

An anti-LC/A or LC/B BoNT VHH polypeptide that specifically binds to and neutralizes the activity of LC/A or LC/B BoNT, may also be referred to as a “VHH-based neutralizing agent (VNA)” a “VNA polypeptide or protein,” a “VNA binding molecule,” or a single-domain variable heavy-chain (VHH) antibody.

As used herein, the terms “treat,” treating,” “treatment,” and the like refer to reducing, diminishing, abating, alleviating, improving, or ameliorating a disorder and/or symptoms associated therewith. It will be appreciated that, although not precluded, treating a disorder or condition does not require that the disorder, condition or symptoms associated therewith be completely eliminated.

The term “multimeric binding molecule” refers in general to a multi-component protein or polypeptide containing two (e.g., dimeric) or more, same or different, VHH binding molecules, which are coupled or linked, e.g., via spacer sequences, to each other and/or other components of the molecule. Multimeric binding molecules may be dimeric, in that the binding molecule contains two VHH polypeptides that bind to LC/A or LC/B BoNT. The anti-LC/A or LC/B VHH polypeptides in a dimeric multimer may be the same or they may be different VHH polypeptides. A dimeric binding molecule may include two anti-LC/A VHH polypeptides that are the same or different. A dimeric binding molecule may include two anti-LC/B VHH polypeptides that are the same or different. A dimeric binding molecule may include one anti-LC/A VHH polypeptide and one anti-LC/B VHH polypeptide. The different anti-LC/A or anti-LC/B VHH polypeptides in a multimeric binding molecule may bind to different regions, portions, or epitopes (e.g., non-overlapping epitopes) of LC/A or LC/B. Alternatively, the multimeric binding molecules may be heteromultimeric, in that the binding molecule contains more than one or two, e.g., three or four, etc. different anti-LC/A or anti-LC/B VHH polypeptides such as described herein. In some embodiments, a heteromultimeric binding molecule contains two, three, four or more different anti-LC/A or anti-LC/B VHH polypeptides, each of which specifically binds to a LC/A or LC/B BoNT, e.g., at different or non-overlapping epitopes. In embodiments, dimeric multimers and heteromultimeric binding molecules comprising two or more anti-LC/A or LC/B VHHs bind to and neutralize the activity of the neurotoxins.

As used herein, the terms “prevent,” “preventing,” “prevention,” “prophylactic treatment,” “protection” and the like refer to reducing the probability of developing a disorder or condition in a subject, who does not have, but who is at risk of, is susceptible to, or disposed to (e.g., genetically disposed to), developing a disease, disorder, pathology, or condition.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, inclusive of the first and last values.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Although various features of the present disclosure can be described in the context of a single embodiment, the features can also be provided in separate embodiments, or in any suitable combination or combination of embodiments. The section headings used herein are for organizational purposes only and are not intended to be limiting to the subject matter described.

The features of the present disclosure are set forth with particularity in the appended claims. The features and advantages of the present disclosure will be better understood and obtained by reference to the detailed description infra, which sets forth illustrative embodiments, in which the principles of the disclosure are utilized, and in view of the accompanying drawings as described herein.

### BRIEF DESCRIPTION OF THE DRAWINGS

FIGS. 1A-1F—Overall structures of VHHs in complex with LC/A. FIG. 1A shows the structures of LC/A-VHH complexes. Inhibitor VHHs are represented in surface. Non-inhibitor VHHs (ciA-D12 and CiA-F12) are shown as ribbon. Duplicated VHH structures are omitted from the figure for clarity. FIGS. 1B-1D: The structures of the JPU-B9-bound fLC/A (FIG. 1B), and the JPU-C10-bound fLC/A (FIG. 1C), and LC/A in BoNT/A holotoxin (PDB code 3BTA) (FIG. 1D). The 210-, 250-, 370-loops and C-terminal peptide of LC/A are highlighted in green, pink, magenta, and orange, respectively. (FIG. 1E): Superposition of the JPU-C10-bound fLC/A with SNAP25(197-202)-bound LC/A (PDB code: 3DDA) highlighting the potential interaction between the C-terminal residues of fLC/A with P5′ M202 (shown as stick) of SNAP25. LC/A is colored as that shown in (B-D) while a short SNAP25 peptide is colored wheat. The interacting residues between the C-terminal peptide with the surrounding loops on LC/A are drawn as sticks. (FIG. 1F): The structure of the LC/A-SNAP25 (146-204) complex (PDB 1D: IXTG), where SNAP25 is shown as an outer ribbon (purple) model. The approximate regions of the α- and β-exosites and the anchoring sites I and II are indicated by dotted circles.

FIGS. 2A-2H: JPU-A5, JPU-C10, and JPU-B9 inhibit the binding of SNAP25 to the β-exosite of LC/A. FIGS. 2A, 2D, and 2G present cartoon representations of JPU-A5 (FIG. 2A), JPU-C10 (FIG. 2D), JPU-B9 (FIG. 2G) in complex with LC/A. The CDR1, 2, and 3 are colored yellow, orange, and magenta, respectively. The framework region (FR) of JPU-A5, JPU-C10, and JPU-B9 are colored cyan, pink, and light green, respectively. (FIGS. 2B, 2E, 2H). Superposition of LC/A-JPU-A5 (FIG. 2B), -JPU-C10 (FIG. 2E), or JPU-B9 (FIG. 2H) with SNAP25(197-202)-bound LC/A (PDB ID: 3DDA). Only the VHH-bound LC/A colored white is represented in (FIG. 2B) and (FIG. 2E) for clarity, while SNAP-25 peptide is colored wheat. In FIG. 2H, both the JPU-B9- and SNAP25-bound LC/A colored white and light yellow, respectively, are shown to illustrate that the JPU-B9-bound conformation is not favorable for SNAP25 binding. JPU-B9 that does not directly compete with SNAP25 is not shown here for clarity. JPU-A5 (FIG. 2C) and JPU-C10 (FIG. 2F) compete with SNAP25 residues for LC/A binding. Molecular surface of LC/A is presented to highlight the binding epitopes. The VHH-binding epitopes on LC/A are colored blue, while the regions overlapping with SNAP25 binding are colored green. Key VHH residues that interfere with SNAP25 binding and the corresponding interacting LC/A residues are drawn as sticks.

FIGS. 3A-3F: JPU-C1 and JPU-G3 block the interaction of SNAP25 to the anchoring site II of LC/A. FIGS. 3A and 3D present cartoon representations of JPU-C1 (FIG. 3A) or JPU-G3 (FIG. 3D) in complex with LC/A. The framework region of JPU-C1 and JPU-G3 are colored cyan and pink respectively. The CDR1, 2, and 3 are colored yellow, orange, and magenta, respectively. FIGS. 3B and 3E: Superposition of LC/A-JPU-C1 (FIG. 3B), -JPU-G3 (FIG. 3E) with SNAP25(146-204)-bound LC/A (PDB ID: 1XTG). Only VHH-bound LC/A is represented. LC/A and the long SNAP25 peptide are colored white and wheat, respectively. (FIGS. 3C, 3F). The VHH residues competing with SNAP25 residues for LC/A binding are drawn as sticks. The epitopes are identified similarly to those shown in FIG. 2.

FIGS. 4A-4F: JPU-D12 and JPU-A11 block the interaction of SNAP25 to the anchoring site I of LC/A. FIGS. 4A and 4D present cartoon representations of JPU-D12 (FIG. 4A) or JPU-A11 (FIG. 4D) in complex with LC/A. The framework region of JPU-D12 and JPU-A11 are colored cyan and pink respectively. The CDR1, 2, and 3 are colored yellow, orange, and magenta, respectively. FIGS. 4B, 4E present superimpositions of LC/A-JPU-C1 (FIG. 4B), -JPU-G3 (FIG. 4E) with SNAP25(146-204)-bound LC/A (PDB ID: 1XTG). Only VHH-bound LC/A is represented. LC/A and SNAP25 are colored as that shown in FIG. 3. (FIGS. 4C, 4F): The VHH residues competing with SNAP25 residues for LC/A binding are drawn as sticks. The epitopes are identified similarly to those shown in FIG. 2.

FIGS. 5A-5F: ALc-H7 and ALc-B8 block the interaction of SNAP25 to the α-exosite of LC/A. FIGS. 5A and 5D present cartoon representations of ALc-H7 (FIG. 5A), ALc-B8 (FIG. 5D) in complex with LC/A. The framework region of ALc-H7 and ALc-B8 are colored cyan and pink, respectively. The CDR1, 2, and 3 are colored yellow, orange, and magenta, respectively. FIGS. 5B and 5E: Superimposition of LC/A-ALc-H7 (FIG. 5B), -ALc-B8 (FIG. 5E) with SNAP25(146-204)-bound LC/A (PDB ID: 1XTG). Only VHH-bound LC/A is represented. LC/A and SNAP25 are colored as that shown in FIG. 3. (FIGS. 5C and 5F): The VHH residues competing with SNAP25 residues for LC/A binding are drawn as sticks. The epitopes are colored as that shown in FIG. 2.

FIGS. 6A-6H: JSG-C1 and JLJ-G3, but not JNE-B10, inhibit the binding of VAMP2 to LC/B. FIG. 6A shows the structure of BoNT/B (PDB code: 1EPW). Only LC/B (limon cartoon) and the heavy chain belt (wheat ribbon) are shown. (right) Superimposition of JSG-C1 (orange), JLJ-G3 (light green), and JNE-B10 (dark green) on fLC/B model. (FIG. 6B-611): JNE-B10, JLJ-G3, and JSG-C1 occupy three distinct binding epitopes on LC/B. Cartoon representations of JSG-C1 (FIG. 6B), JLJ-G3 (FIG. 6E), and JNE-B10 (FIG. 6G) in complex with LC/B. The framework region of JSG-C1, JLJ-G3, and JNE-B10 are colored cyan, pink, and light green, respectively. The CDR1, 2, and 3 are colored yellow, orange, and magenta, respectively. Superposition of LC/B-JSG-C1 (FIG. 6C), -JLJ-G3 (FIG. 6F) and -JNE-B10 (FIG. 6H) with BoNT/B. Only VHH-bound LC/B and the heavy chain belt (wheat) of BoNT/B are drawn. The VHH-binding epitopes on LC/B are colored blue. Potential JSG-C1 residues that compete with VAMP2 residues for LC/B binding are represented as sticks (FIG. 6D).

FIGS. 7A-7C: Sequence conservation of VHH-binding epitopes on LC across BoNT/A or BoNT/B subtypes. FIGS. 7A and 7B present the overall sequence identity (grey) and similarity (black) among BoNT/A or BoNT/B subtypes using A1 and B1 as the benchmarks. FIG. 7C shows the conservation of epitope sequences for selected VHHs. In FIG. 7C, A2 on the x-axis in the graphs of ALc-B8, ALc-H7, JPU-A11, JPU-D12, JPU-C1, JPU-G3, JPU-A5, and JPU-C10; A4 on the x-axis in the graphs of ALc-H7, JPU-A11, JPU-D12, JPU-A5 and JPU-C10; A3 on the x-axis in the graphs of JPU-C1, JPU-G3, JPU-A5 and JPU-C10; and B8 on the x-axis of the graphs of JSG-C1 and JLJ-G3 are denoted as indicating comparable VHH binding to that specific subtype in comparison to A1 or B1. In contrast, A3 on the x-axis in the graphs of ALc-B8, ALc-H7, JPU-A11 and JPU-D12, and A4 on the x-axis in the graphs of ALc-B8, JPU-C1 and JPU-G3 are labeled as indicating weakened binding.

FIG. 8: Sequence alignment of LC/A-binding VHHs. FIG. 8 presents the amino acid sequences of all of the LC/A-binding VHHs as described herein. Sequences are aligned to show conserved framework regions, and CDRs are indicated. Note that the amino acids encoding the amino terminal six amino acids of the VHH framework 1 are not shown, as they are altered during PCR to amplify the VHH coding DNA during VHH-display phage library construction. In FIG. 8, the amino acid sequence of VHH ALc-B8 is designated SEQ ID NO: 1; the amino acid sequence of VHH ALc-H7 is designated SEQ ID NO: 2; the amino acid sequence of VHH ciA-D1 is designated SEQ ID NO: 3; the amino acid sequence of VHH ciA-D12 is designated SEQ ID NO: 4; the amino acid sequence of VHH ciA-F12 is designated SEQ ID NO: 5; the amino acid sequence of VHH ciA-H7 is designated SEQ ID NO: 6; the amino acid sequence of VHH JPU-A1 is designated SEQ ID NO: 7; the amino acid sequence of VHH JPU-A5 is designated SEQ ID NO: 8; the amino acid sequence of VHH JPU-A11 is designated SEQ ID NO: 9; the amino acid sequence of VHH JPU-B5 is designated SEQ ID NO: 10; the amino acid sequence of VHH JPU-B9 is designated SEQ ID NO: 11; the amino acid sequence of VHH JPU-C1 is designated SEQ ID NO: 12; the amino acid sequence of VHH JPU-C10 is designated SEQ ID NO: 13; the amino acid sequence of VHH JPU-D12 is designated SEQ ID NO: 14; the amino acid sequence of VHH JPU-G3 is designated SEQ ID NO: 15; the amino acid sequence of VHH JPU-G7 is designated SEQ ID NO: 16; the amino acid sequence of VHH JPU-G11 is designated SEQ ID NO: 17; the amino acid sequence of VHH JPU-G12 is designated SEQ ID NO: 18; and the amino acid sequence of VHH JPU-H7 is designated SEQ ID NO: 19. In embodiments, the products, compositions and methods described herein include one or more of the following anti-LC/A toxin VHH polypeptides: JPU-A1 (SEQ ID NO: 7); JPU-A5 (SEQ ID NO: 8); JPU-A11 (SEQ ID NO: 9); JPU-B5 (SEQ ID NO: 10); JPU-B9 (SEQ ID NO: 11); JPU-C1 (SEQ ID NO: 12); JPU-C10 (SEQ ID NO: 13); JPU-D12 (SEQ ID NO: 14); JPU-G3 (SEQ ID NO: 15); JPU-G7 (SEQ ID NO: 16); JPU-G11 (SEQ ID NO: 17); JPU-G12 (SEQ ID NO: 18); or JPU-H7 (SEQ ID NO: 19). The amino acid sequences of the BoNT LC/A-binding VHH polypeptides shown and identified in FIG. 8 include the three CDRs (CDRs 1-3) and four framework (FRs 1-4) sequences, as shown, of each of the VHH polypeptides.

FIG. 9: Sequence alignment of LC/B-binding VHHs. FIG. 9 presents the amino acid sequences of all of the LC/B binding VHHs as described herein. Sequences are aligned to conserved framework regions, and CDRs are indicated. Note that the amino acids encoding the amino terminal six amino acids of the VHH framework 1 are not shown as they are altered during PCR to amplify the VHH coding DNA during VHH-display phage library construction. In FIG. 9, the amino acid sequence of VHH BLc-B10 is designated SEQ ID NO: 20; the amino acid sequence of VHH JLJ-F9 is designated SEQ ID NO: 21; the amino acid sequence of VHH JLJ-G3 is designated SEQ ID NO: 22; the amino acid sequence of VHH JNE-B10 is designated SEQ ID NO: 23; the amino acid sequence of VHH JND-A12 is designated SEQ ID NO: 24; the amino acid sequence of VHH JND-B4 is designated SEQ ID NO: 25; the amino acid sequence of VHH JND-C7 is designated SEQ ID NO: 26; the amino acid sequence of VHH JND-E4 is designated SEQ ID NO: 27; the amino acid sequence of VHH JND-E5 is designated SEQ ID NO: 28; the amino acid sequence of VHH JND-E9 is designated SEQ ID NO: 29; the amino acid sequence of VHH JND-F3 is designated SEQ ID NO: 30; the amino acid sequence of VHH JSG-B8 is designated SEQ ID NO: 31; the amino acid sequence of VHH JSG-B10 is designated SEQ ID NO: 32; the amino acid sequence of VHH JSG-C1 is designated SEQ ID NO: 33; the amino acid sequence of VHH JSG-F6 is designated SEQ ID NO: 34; the amino acid sequence of VHH JSG-G1 is designated SEQ ID NO: 35; the amino acid sequence of VHH JSG-G10 is designated SEQ ID NO: 36; and the amino acid sequence of VHH JSG-G11 is designated SEQ ID NO: 37. In embodiments, the products, compositions and methods described herein include one or more of the following anti-LC/B toxin VHH polypeptides: JND-A12 (SEQ ID NO: 24); JND-B4 (SEQ ID NO: 25); JND-C7 (SEQ ID NO: 26); JND-E4 (SEQ ID NO: 27); JND-E5 (SEQ ID NO: 28); JND-E9 (SEQ ID NO: 29); JND-F3 (SEQ ID NO: 30); JSG-B8 (SEQ ID NO: 31); JSG-B10 (SEQ ID NO: 32); JSG-C1 (SEQ ID NO: 33); JSG-F6 (SEQ ID NO: 34); JSG-G1 (SEQ ID NO: 35); JSG-G10 (SEQ ID NO: 36); and JSG-G11 (SEQ ID NO: 37). The amino acid sequences of the BoNT LC/B-binding VHH polypeptides shown and identified in FIG. 9 include the three CDRs (CDRs 1-3) and four framework (FRs 1-4) sequences, as shown, of each of the VHH polypeptides.

FIG. 10: Western blot assessment of VHH potency to inhibit LC/A protease activity. LC/A protease was incubated with the BoTest A/E reporter in the presence or absence of VHHs at a molar ratio of 5:1 VHH:LC/A. Incubations were performed at 37° C. for either 10 or 60 minutes as indicated, and the reaction was terminated by boiling in SDS sample buffer. An equal aliquot of each sample was analyzed by performing western blots and the substrate and products were detected by HRP/anti-GFP antibodies.

FIG. 11: Western blot assessment of VHH potency to inhibit LC/B protease activity. LC/B protease was incubated with a recombinant YFP/VAMP/GFP reporter in the presence or absence of VHHs at a molar ratio of 2:1 VHH:LC/A. Incubations were performed at 37° C. for either 60 or 180 minutes as indicated, and the reaction was terminated by boiling in SDS sample buffer. An equal aliquot of each sample was analyzed by performing western blots and the substrate and products were detected by HRP/anti-GFP antibodies.

FIG. 12A-12J: Structural predictions as to cross-specificity of VHHs to bind to the known LC/A or LC/B subtypes. Sequence conservation of VHH-binding epitopes across BoNT/A or BoNT/B subtypes (FIGS. 12A-12H) LC/A or LC/B (FIGS. 12I and 12J) is drawn in surface. Identical, conserved, semi-conserved, and variable residues at the LC-VHH interface are colored, green, blue, purple, and red, respectively. (Right) The amino acid sequence alignment among BoNT/A or BoNT/B subtypes was performed using Clustal Omega. Only the VHH-binding epitopes are represented. FIGS. 12A-12J disclose SEQ ID NOs: 130, 130-133, 130, 134, 130, 135-139, 135, 135, 135, 140, 140-142, 140, 140, 140, 140, 143, 143-146, 143, 143, 147-151, 149, 148, 152-157, 155, 154-155, 158-159, 159, 159-160, 159, 159, 161, 159, 162, 162, 162-163, 162, 162, 164, 162, 165, 165, 165-167, 165, 165, 165, 168, 168, 168, 168, 168, 168, 168, and 168, respectively, in order of appearance.

FIG. 13: Dilution ELISAs to assess the cross-specificity of VHHs for selected LC/A or LC/B subtypes. Recombinant LC/A1, /A2, /A3 and/A4 and LC/B1 and/B8, each of which was a strep-tag fusion protein, were captured onto streptactin plates, and VHH binding dilution ELISAs were performed as described in the Methods. The name of the VHH tested in each ELISA plot is indicated above the data. VHH binding data to each subtype is indicated in and plotted as a function of VHH concentration.

FIG. 14 presents a Table showing anti-LC/A and LC/B toxin VHH data collection and refinement statistics as described in Examples 2 and 4.

FIG. 15 presents a Table showing anti-LC/A and LC/B toxin VHH data and statistics as described in Example 4.

### DETAILED DESCRIPTION OF THE EMBODIMENTS

Botulinum neurotoxins (BoNTs) are extremely toxic to humans by causing flaccid paralysis of botulism. The catalytic light chain (LC) of BoNTs is the warhead of the toxin, which is mainly responsible for BoNT's neurotoxic effects. As an endopeptidase, LC is delivered by the toxin to inside neurons where it specifically cleaves neuronal SNARE proteins and causes muscle paralysis. While the currently available equine antitoxin sera could prevent further intoxication, they do not promote recovery from paralysis that has already occurred. Described herein are single-domain variable heavy-chain (VHH) antibodies developed to target the LC of BoNT/A (LC/A) and BoNT/B (LC/B). Such VHHs are advantageously useful as antidotes to inhibit or eliminate the intraneuronal LC protease. A panel of VHHs that bind to LC/A or LC/B has been identified and characterized as described herein. Using a combination of X-ray crystallography and biochemical assays, it was found that VHHs exploit diverse mechanisms to interact with LC/A and LC/B and inhibit their protease activity, and such knowledge can be harnessed to predict their specificity toward different toxin subtypes within each serotype. The new VHHs and their characterization reported herein can contribute to the development of improved botulism therapeutics having high potencies and broad specificities.

The VHHs described herein can be considered to be the next generation of botulism antidotes with the goal to inhibit or eliminate the intraneuronal protease that causes paralysis. In an embodiment, the single-domain antibodies (sdAbs; VHHs) developed and characterized as described herein can be employed to target BoNT protease toxins and to treat an intoxicated subject. The VHHs can also be components of pharmaceutical compositions employed as biomolecular antidotes that target the various BoNT proteases. The terms sdAbs and VHHs are used interchangeably herein.

The described sdAbs (also termed VHHs or VHH polypeptides herein) are derived from the heavy-chain-only antibodies produced by camelid animals. The heavy chain variable regions of these antibodies, called VHHs or nanobodies, are small sdAbs of ˜14 kDa that are tightly folded, stable, and highly amenable to recombinant expression and engineering as multimers or as fusions to other vehicles. VHHs also have a preference for binding to conformational epitopes that often are functional sites on proteins such as enzyme active sites or receptor-binding sites. Multimers of toxin-neutralizing VHHs have been employed in the generation of botulism antitoxins and have been shown to have substantially improved potencies [6, 7], particularly when the multimer designs are guided by structural information [8].

The VHHs described herein bind to LC/A or LC/B of botulinum toxin A or B, many having potent activities to inhibit the protease function. To gain insight into the mechanisms of neutralization, structural studies were performed to identify the protease binding sites for many of the VHHs. The structural information also reveals the potential of these VHHs to bind and neutralize other known natural subtypes of these two proteases. The VHHs and their characterization described herein contributes to the development of improved botulism therapeutics having higher potencies and broader specificities than those reported to date.

The anti-BoNT LC/A VHH polypeptides or multimeric forms thereof and anti-BoNT LC/B VHH polypeptides as described herein are provided as beneficial therapeutic agents and/or antitoxins that bind to the light chains of neurotoxins produced by C. botulinum and other species. In some cases, anti-BoNT LC/A VHH polypeptides or multimeric forms thereof and anti-BoNT LC/B VHH polypeptides or multimeric forms thereof promote toxin neutralization by rapidly and effectively blocking further toxin activity and also accelerate clearance of toxin from the system to eliminate future pathology.

In some embodiments, the binding activity and/or neutralizing activity of the LC/A-binding VHH polypeptides and LC/B-binding VHH polypeptides described herein, in the absence of any epitope tag sequences are significantly effective such that the antitoxin function of these molecules obviates the need for an anti-tag antibody or clearing antibody.

VHHs, such as the LC/A-binding VHH polypeptides and LC/B-binding VHH polypeptides described herein, have a number of advantages over conventional antibodies and recombinant antibody domains, including (i) they are small monomeric proteins (14 kDa) that express and fold efficiently in recombinant hosts; (ii) they are more stable to extremes of pH and temperature compared with conventional antibodies; (iii) they typically bind conformational epitopes, and thus are more likely to neutralize target functions; and (iv) they are amenable to designed multimerization which often leads to higher potencies and a reduction in the risk that microorganisms (e.g., C. botulinum) will develop resistance; and (v) they offer more therapeutic versatility, such as multispecificity, thus supporting their beneficial utility in treating botulism and intoxication by botulinum toxin.

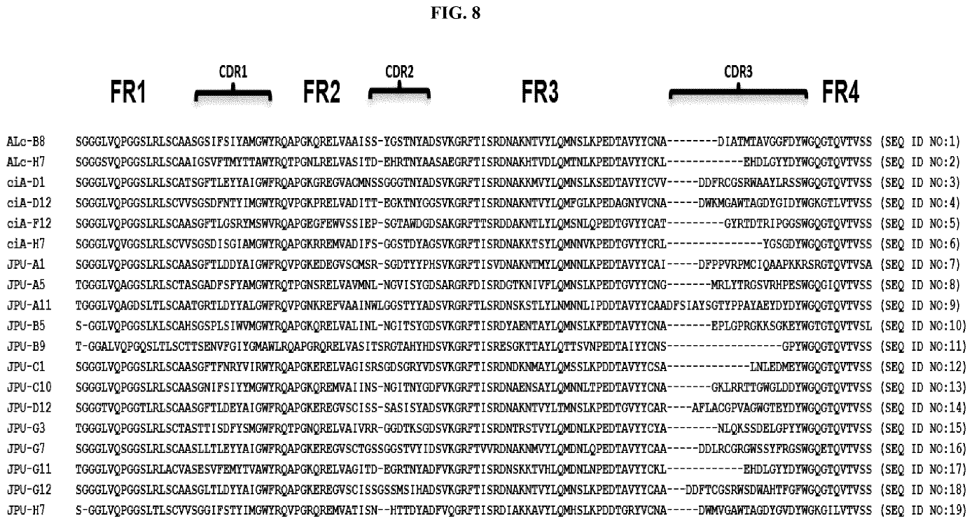

The amino acid sequences of representative anti-LC/A toxin VHH polypeptides (antibodies) described herein are set forth, for example, in SEQ ID NOs: 7-19. The toxin binding CDR sequences of these anti-LC/A toxin VHH polypeptides are set forth in Table 1 (SEQ ID NOs: 38-76). In an embodiment, an LC/A toxin-binding polypeptide having at least 85%, at least 88%, at least 90%, at least 93%, at least 95%, at least 96%, at least 97%, at least 98%, at least 99% or greater, sequence identity, (as well as percentages therebetween) amino acid sequence identity to one or more of the LC/A toxin-binding VHH polypeptides of SEQ ID NOs: 7-19 and FIG. 8 is encompassed. In an embodiment, the anti-LC/A toxin VHH polypeptide is JPU-A5 (SEQ ID NO: 8) or JPU-C10 (SEQ ID NO: 13), which bind to the (3-exosite of LC/A toxin, or a polypeptide having at least 85%, at least 88%, at least 90%, at least 93%, at least 95%, at least 96%, at least 97%, at least 98%, at least 99% or greater, sequence identity, (as well as percentages therebetween) amino acid sequence identity to the JPU-A5 or the JPU-C10 amino acid sequence. Also encompassed are polynucleotides encoding the anti-LC/A toxin VHH polypeptides described herein.

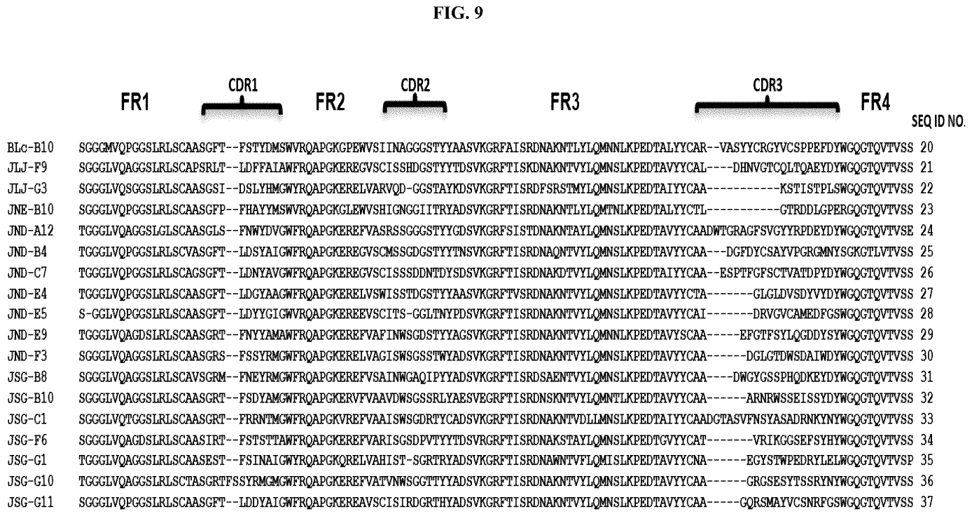

The amino acid sequences of representative anti-LC/B toxin VHH polypeptides (antibodies) described herein are set forth, for example, in SEQ ID NOs: 24-37. The toxin binding CDR sequences of these anti-LC/B toxin VHH polypeptides are set forth in Table 2 (SEQ ID NOs: 77-118). In an embodiment, an LC/B toxin-binding polypeptide having at least 85%, at least 88%, at least 90%, at least 93%, at least 95%, at least 96%, at least 97%, at least 98%, at least 99% or greater, sequence identity, (as well as percentages therebetween) amino acid sequence identity to one or more of the LC/B toxin-binding VHH polypeptides of SEQ ID NOs: 24-37 and FIG. 9 is encompassed. In an embodiment, the anti-LC/B toxin VHH polypeptide is JSG-C1 (SEQ ID NO: 33) or a polypeptide having at least 85%, at least 88%, at least 90%, at least 93%, at least 95%, at least 96%, at least 97%, at least 98%, at least 99% or greater, sequence identity (as well as percentages therebetween) amino acid sequence identity to the amino acid sequence of JSG-C1. Also encompassed are polynucleotides encoding the anti-LC/B toxin VHH polypeptides described herein.

By way of non-limiting example, the binding of CDRs to a target protein (toxin) may be via conformational binding or interaction, electrostatic binding interaction, hydrogen bonding, Van der Waals forces, or hydrophobic bonding, or combinations thereof, as would be appreciated by those having skill in the art.

### Botulism and Botulinum Toxin (BoNT)

Botulism is a rare but serious illness caused by a neurotoxin that attacks the cells and tissues of the nervous system and causes difficulty breathing, muscle paralysis, and even death in humans and other animals. This toxin is produced by bacterial microorganisms, for example, Clostridium botulinum, Clostridium butyricum, and Clostridium baratii. Clostridium botulinum (C. botulinum) is a Gram-positive, rod-shaped, anaerobic, spore-forming, motile bacterium that has the ability to produce the disease-causing neurotoxins (toxins). Botulinum toxin, whether natural or synthetic, is highly potent, as it is associated with a lethal dose of 1.3-2.1 ng/kg in humans. C. botulinum constitutes a diverse group of pathogenic bacteria initially grouped together by their ability to produce botulinum toxin and now known as four distinct groups, C. botulinum groups I-IV.

C. botulinum is responsible for foodborne botulism (ingestion of preformed toxin), infant botulism (intestinal infection with toxin-forming C. botulinum), and wound botulism (infection of a wound with C. botulinum). C. botulinum produces heat-resistant endospores that are commonly found in soil and are able to survive under adverse conditions.

Botulinum toxin, often shortened to “BoNT,” is a neurotoxic protein produced by Clostridium botulinum and related species. The toxin prevents the release of the neurotransmitter acetylcholine from axon endings at the neuromuscular junction, thus causing flaccid paralysis, and is the causative agent of botulism disease. The toxin is also used commercially for medical and cosmetic purposes. The seven main types of botulinum toxin are types (or subtypes) A to G (A, B, C1, C2, D, E, F and G). New types are occasionally found, Types A and B are capable of causing disease in humans, and are also used commercially and medically. Types C-G are less common; types E and F can cause disease in humans, while the other toxin types cause disease in other animals. Botulinum toxin types A and B are used in medicine to treat various muscle spasms.

Botulinum toxins are among the most potent toxins known. Intoxication can occur naturally as a result of either wound or intestinal infection, or by ingesting formed toxin in foods. The estimated human lethal dose of type A toxin is 1.3-2.1 ng/kg intravenously or intramuscularly, 10-13 ng/kg via inhalation, or 1000 ng/kg via ingestion or when taken orally. Commercial forms of botulinum toxin are marketed under the brand names Botox (onabotulinumtoxinA), Dysport/Azzalure (abobotulinumtoxinA), Xeomin/Bocouture (incobotulinumtoxinA), and Jeuveau (prabotulinumtoxinA).

### Therapeutic Methods

Botulinum neurotoxin (BoNT) serotypes A and B, in particular, pose the most serious threats to humans because of their high potency and persistence. To date, there is no effective treatment for post-exposure or late post-exposure therapy to provide for patients suffering from botulism.

Featured herein are methods for treating or preventing disease and intoxication caused by the LC/A and LC/B toxins produced by botulinum bacteria such as C. botulinum following infection of a subject by the bacteria. The methods include administering to a subject in need thereof an effective amount (e.g., a therapeutically effective amount) of an anti-LC/A toxin and/or an anti-LC/B toxin VHH polypeptide or a multimeric form thereof that is effective to specifically bind to and optimally neutralize the toxins, so as to treat the disease, illness, condition, intoxication and/or symptoms thereof. In an embodiment, if an anti-LC/A toxin VHH polypeptide or multimeric form thereof and/or an anti-LC/B toxin VHH polypeptide or multimeric form thereof includes an epitope tag as described further below, an anti-epitope tag antibody may also be administered to the subject (see, e.g., WO 2019/094095A1, the contents of which is incorporated by reference herein in its entirety).

Methods of treating a subject infected with a botulinum microorganism that produces deadly neurotoxins involve the administration of an effective amount of one or more single-domain variable heavy-chain (VHH) polypeptides (also called sdAbs or VHH antibodies) that target and bind to the protease domains (also known as the light chain, LC) of BoNT/A and BoNT/B to inhibit, block, or neutralize toxin activity, and to serve as antidotes for post-intoxication (post-infection and disease) treatments and therapy. The VHHs recognize four and three non-overlapping epitopes on the LC of BoNT/A and BoNT/B, respectively. Upon binding, the VHHs inhibit the LC activity as a result of their occupying the extended substrate-recognition exosites or the cleavage pocket of LC/A or LC/B, thereby blocking substrate binding. Several of the VHHs recognize highly conserved epitopes across BoNT/A or BoNT/B subtypes, suggesting that these VHHs exhibit broad botulinum subtype efficacy. In addition, two novel conformations of the full-length LC/A were identified and could be targets for inhibitors of BoNT/A.

The anti-BoNT LC/A and anti-BoNT LC/B VHH polypeptides (antibodies) may be administered to a subject as monomers or as multimers including two, three, or four VHH polypeptides, for example. In an embodiment, the method involves administering one or more anti-BoNT LC/A VHH polypeptides to a subject in need, for example, a subject who is intoxicated following infection and disease caused by a botulinum bacterium that produces neurotoxin, such as subtype A neurotoxin. In an embodiment, the method involves administering one or more anti-BoNT LC/B VHH polypeptides to a subject in need, for example, a subject who is intoxicated following infection and disease caused by a botulinum bacterium that produces neurotoxin, such as subtype B neurotoxin. In an embodiment, a combination of one or more of the anti-BoNT LC/A VHH polypeptides and the anti-BoNT LC/B VHH polypeptides can be administered to a subject in need, for example, a subject who is intoxicated with botulinum bacteria producing subtype A and/or subtype B toxins to inhibit the toxicity of the neurotoxin(s). In an embodiment, the method involves administering an LC/A-binding polypeptide having at least 85%, at least 88%, at least 90%, at least 93%, at least 95%, at least 96%, at least 97%, at least 98%, at least 99% or greater, sequence identity (and percentages therebetween) amino acid sequence identity to one or more of the LC/A toxin-binding VHH polypeptides of SEQ ID NOs: 7-19 as described herein, or a polynucleotide encoding the polypeptide, to the intoxicated subject. In an embodiment, the method involves administering an LC/A-binding VHH polypeptide, namely, one or more of the LC/A-binding VHH polypeptides of SEQ ID NOs: 7-19 as described herein, or a polynucleotide encoding the polypeptide, to the intoxicated subject. In an embodiment, the method involves administering an LC/A-binding VHH polypeptide, namely, one or more of the LC/A-binding VHH polypeptides of SEQ ID NOs: 7-19 as described herein, or a polynucleotide encoding the polypeptide, to the intoxicated subject. In an embodiment, the method involves administering to an intoxicated subject (e.g., a patient infected by an LC/A toxin-producing botulinum bacterium) an LC/A-binding VHH polypeptide, namely, an LC/A-toxin binding VHH polypeptide containing three CDRs that bind to LC/A toxin, such as the CDRs of SEQ ID NOs: 38-76 as described herein (Table 1), or a polynucleotide encoding the polypeptide.

In an embodiment, the method involves administering to an intoxicated subject (e.g., a patient infected by an LC/B toxin-producing botulinum bacterium) an LC/B-binding VHH polypeptide, namely, one or more of the LC/B toxin-binding VHH polypeptides of SEQ ID NOs: 24-37 as described herein, or a polynucleotide encoding the polypeptide. In an embodiment, the method involves administering an LC/B-binding VHH polypeptide, namely, an LC/B-toxin binding VHH polypeptide containing three CDRs that bind to LC/B toxin, such as the CDRs of SEQ ID NOs: 77-118 as described herein (Table 2), or a polynucleotide encoding the polypeptide, to the intoxicated subject. In an embodiment, the method involves administering an LC/B toxin-binding polypeptide having at least 85%, at least 88%, at least 90%, at least 93%, at least 95%, at least 96%, at least 97%, at least 98%, at least 99% or greater, sequence identity (and percentages therebetween) amino acid sequence identity to one or more of the LC/B toxin-binding VHH polypeptides of SEQ ID NOs: 24-37 as described herein, or a polynucleotide encoding the polypeptide, to the intoxicated subject.

In an embodiment, the LC/A toxin-binding and LC/B toxin-binding VHH polypeptides can be produced in monomeric or multimeric forms for use in the treatment and therapeutic methods. Multimeric forms of the VHH polypeptides include dimers, trimers, and tetramers that are linked, joined, or coupled by flexible linker peptides known in the art (see, e.g., WO 2022/006219A2). In an embodiment, the multimeric VHH polypeptides can contain the same or different VHH polypeptides that bind to LC/A toxin. In an embodiment, the multimeric VHH polypeptides can contain the same or different VHH polypeptides that bind to LC/B toxin. In an embodiment, the multimeric VHH polypeptides can contain a combination of VHH polypeptides that bind to LC/A toxin and VHH polypeptides that bind to LC/B toxin. By way of example, multimeric forms of VHH polypeptides are described, for example, in U.S. Pat. Nos. 10,766,950; 11,091,563; and U.S. Publication No. 2021/0221874, the contents of which are incorporated by reference herein.

In other embodiments, the monomeric or multimeric LC/A toxin-binding and LC/B toxin-binding VHH polypeptides may be linked to an epitope tag (or E-tag), which can be bound by an anti-E-tag antibody (monoclonal antibody (Mab)), e.g., to aid in clearance of complexes containing a VHH binding polypeptide and bound toxin from the body. Such epitope tags are used in the art and are described, for example, in U.S. Pat. Nos. 8,349,326; 8,865,169; 10,766,950; U.S. Publication No. 2021/0221874; and WO 2022/006219A2, the contents of which are incorporated by reference herein. In an embodiment, an LC/A-binding VHH polypeptide or an LC/B-binding polypeptide is recombinantly produced and is a recombinant protein.

In an aspect, according to the methods of treatment described herein, administration (or immunization) involves contacting a subject with a pharmaceutical composition containing an LC/A toxin-binding and/or LC/B toxin-binding VHH polypeptide or multimeric form thereof, as described herein. Thus, methods are provided for immunization, comprising administering to a subject in need thereof, such as a subject infected with C. botulinum or a subject having botulism or symptoms related to botulism, a therapeutically effective amount of a pharmaceutical composition comprising an LC/A toxin-binding VHH polypeptide and/or LC/B toxin-binding VHH polypeptide or multimeric form thereof as active agent for a time necessary to achieve the desired result. It will be appreciated that the methods encompass protectively administering the pharmaceutical composition as a preventive or therapeutic measure to ameliorate, reduce, abrogate, diminish, or alleviate intoxication and the effects thereof caused by C. botulinum-produced neurotoxin following infection and intoxication caused by the activity of the LC/A and LC/B neurotoxins produced by the bacterium.

The therapeutic methods include prophylactic as well as therapeutic treatment methods. In an embodiment, the treatment method includes administering a therapeutically effective amount of an LC/A toxin-binding VHH polypeptide and/or LC/B toxin-binding VHH polypeptide or multimeric form thereof as described herein, or a pharmaceutical composition comprising these agents, before or during the time that a subject is administered one or more additional drugs or antibiotics, or a treatment involving a course of drugs or antibiotics, to treat the bacterial disease and infection.

A subject or patient includes an animal, particularly a mammal, and more particularly, a human. Such an LC/A toxin-binding VHH polypeptide and/or LC/B toxin-binding VHH polypeptide or multimeric form thereof as described herein, or a pharmaceutical composition comprising these agents, used as therapeutics in treatments will be suitably administered to subjects or patients suffering from, having, susceptible to, or at risk of becoming afflicted with a disease, disorder, or symptom thereof caused by or associated with infection by C. botulinum or other LC/A toxin and/or LC/B toxin-producing bacteria. Determination of patients who are “susceptible” or “at risk” can be made by any objective or subjective determination obtained by the use of a diagnostic test or based upon the opinion of a patient or a health care provider. Identifying a subject in need of the treatments herein can be in the judgment of a subject him/herself, or of a health care/medical professional and can be subjective (e.g., opinion) or objective (e.g., measurable or quantifiable by a test or diagnostic method).

### Pharmaceutical Compositions

An anti-LC/A toxin and/or an anti-LC/B toxin VHH polypeptide as described herein or multimeric form thereof is provided or contained in a pharmaceutical composition, particularly when used in the practice of the above-described treatment methods.

Typically, a carrier, excipient, or vehicle is included in a composition as described herein, such as a pharmaceutically acceptable carrier, excipient, or vehicle that includes, for example, sterile water, aqueous saline solution, aqueous buffered saline solutions, aqueous sucrose, dextrose, or mannose solutions, aqueous glycerol solutions, ethanol, calcium carbonate, albumin, starch, cellulose, silica gel, polyethylene glycol (PEG), dried skim milk, rice flour, magnesium stearate, and the like, or combinations thereof. The terms “pharmaceutically acceptable carrier” and “carrier” refer to any generally acceptable excipient or drug delivery device that is relatively inert and non-toxic.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's The Science and Practice of Pharmacy Ed. by LWW 21^{st} EQ. PA, 2005 (and current versions) discloses various carriers used in formulating pharmaceutical compositions and known techniques for the preparation thereof. Carriers are selected to prolong dwell time for example following any route of administration, including intraperitoneal (IP), intravenous (IV), subcutaneous (SC), intramuscular (IM), mucosal, sublingual, inhalation or other form of intranasal administration, or other routes of administration, e.g., intradermal, intracranial, intrathecal, intravaginal, rectal, intraorbital, and the like.

Some examples of materials that can serve as pharmaceutically acceptable carriers include, but are not limited to, sugars such as glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The preparation of such compositions and solutions ensuring sterility, pH, isotonicity, and stability is effected according to protocols established in the art. Generally, a carrier or excipient is selected to minimize allergic and other undesirable effects, and to suit the particular route of administration, e.g., subcutaneous, intramuscular, intranasal, intravenous, oral, and the like. In certain embodiments, the compositions optionally further comprise one or more additional therapeutic agents.

A therapeutically effective dose refers to that amount of active agent which ameliorates at least one symptom or condition. Therapeutic efficacy and toxicity of active agents can be determined by standard pharmaceutical procedures in cell cultures or experimental animals, e.g., ED₅₀ (the dose that is therapeutically effective in 50% of the population) and LD₅₀ (the dose that is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices are especially useful. The data obtained from cell culture assays and from animal studies are used in formulating a range of dosages for human administration. By way of example, a therapeutic dose may be at least about 1 μg per kg, at least about 5, 10, 50, 100, 500 μg per kg, at least about 1 mg/kg, 5, 10, 50 or 100 mg/kg body weight of a composition or active component thereof per body weight of the subject, although the doses may be more or less depending on age, health status, history of prior infection, and immune status of the subject as would be known by one of skill in the art. Doses may be divided or unitary and may be administered once daily, or repeated at appropriate intervals.

### Administration of Pharmaceutical Compositions

After formulation with an appropriate pharmaceutically acceptable carrier, excipient, or vehicle in a desired dosage, a pharmaceutical composition comprising an anti-LC/A toxin VHH polypeptide or multimeric form thereof, or an anti-LC/B toxin VHH polypeptide or multimeric form thereof, can be administered to humans and other mammals by routes known and practiced in the art. The administration of an anti-LC/A toxin VHH polypeptide or multimeric form thereof, and/or an anti-LC/B toxin VHH polypeptide or multimeric form thereof, as a therapeutic for the treatment or prevention of intoxication and the symptoms thereof caused by botulism disease may be by any suitable means that results in a concentration of the therapeutic that, combined with other components, if desired, is effective in ameliorating, reducing, eliminating, abating, abrogating, or stabilizing the disease, pathology, intoxication, and/or the symptoms thereof in a subject. The therapeutic may be administered systemically, for example, formulated in a pharmaceutically-acceptable composition or buffer such as physiological saline.

Routes of administration include, for example and without limitation, subcutaneous, intravenous, intraperitoneal, intramuscular, intrathecal, intraperitoneal, or intradermal injections that provide continuous, sustained levels of the therapeutic in the subject. Other routes include, without limitation, intrathecal, intracranial, gastrointestinal, esophageal, oral, rectal, intravaginal, etc. The amount of the therapeutic to be administered varies depending upon the manner of administration, the age and body weight of the subject, and with the clinical symptoms of the bacterial infection, intoxication by neurotoxins, or associated disease, pathology, and/or symptoms. Generally, amounts will be in the range of those used for other agents used in the treatment of disease or pathology associated with C. botulinum infection, although in certain instances, lower amounts may be suitable because of the increased range of protection and treatment afforded by the described anti-LC/A toxin VHH polypeptides or multimeric forms thereof, and anti-LC/B toxin VHH polypeptides or multimeric forms thereof, as therapeutics. A composition is administered at a dosage that ameliorates, decreases, diminishes, abrogates, alleviates, or eliminates the effects of the bacterial infection or disease (e.g., botulism and/or the symptoms thereof) as determined by a method known to one skilled in the art.

In embodiments, a therapeutic or prophylactic treatment agent (e.g., the VHH polypeptides described herein) may be contained in any appropriate amount in any suitable carrier substance, and is generally present in an amount of 1-95% by weight of the total weight of the composition. The composition may be provided in a dosage form that is suitable for parenteral (e.g., subcutaneous, intravenous, intramuscular, intrathecal, or intraperitoneal) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions may in some cases be formulated to release the active agent substantially immediately upon administration or at any predetermined time or time period after administration. The latter types of compositions are generally known as controlled release formulations, which include (i) formulations that create a substantially constant concentration of a therapeutic agent or drug within the body over an extended period of time; (ii) formulations that after a predetermined lag time create a substantially constant concentration of a therapeutic agent or drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with an organ, such as the gut or gastrointestinal system; (v) formulations that allow for convenient dosing, such that doses are administered, for example, once every one or two weeks; and (vi) formulations that target a disease using carriers or chemical derivatives to deliver the therapeutic agent or drug to a particular cell type. For some applications, controlled release formulations obviate the need for frequent dosing during the day to sustain a therapeutic level in plasma, serum, or blood. In an embodiment, one or more anti-LC/A toxin VHH polypeptide or multimeric form thereof, or anti-LC/B toxin VHH polypeptide or multimeric form thereof, may be formulated with one or more additional components for administration to a subject in need, e.g., patients who suffer from infection by C. botulinum or botulism, and suffer from the serious repercussions of toxin production by the infecting bacteria.

Any of a number of strategies can be pursued in order to obtain controlled release of a therapeutic agent in which the rate of release outweighs the rate of metabolism of the therapeutic agent or drug in question. In one example, controlled release is obtained by appropriate selection of various formulation parameters and ingredients, including, e.g., various types of controlled release compositions and coatings. Thus, the therapeutic agent or drug may be formulated with appropriate excipients into a pharmaceutical composition that, upon administration, releases the therapeutic agent or drug in a controlled manner. Examples include single or multiple unit tablet or capsule compositions, oil solutions, suspensions, emulsions, microcapsules, microspheres, molecular complexes, nanoparticles, patches, and liposomes.

Compositions for parenteral or oral use may be provided in unit dosage forms (e.g., in single-dose ampules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent (i.e., an anti-LC/A toxin VHH polypeptide or multimeric form thereof, or an anti-LC/B toxin VHH polypeptide or multimeric form thereof) that reduces or ameliorates intoxication by botulinum toxins, the composition may include suitable parenterally acceptable carriers and/or excipients. The composition may further include suspending, solubilizing, stabilizing, pH-adjusting agents, tonicity adjusting agents, and/or dispersing, agents.

In some embodiments, compositions comprising an anti-LC/A toxin VHH polypeptide or multimeric form thereof, and/or an anti-LC/B toxin VHH polypeptide or multimeric form thereof are sterilized and, if desired, mixed with auxiliary agents, e.g., lubricants, preservatives, stabilizers, wetting agents, emulsifiers, salts for influencing osmotic pressure, buffers, coloring, and/or aromatic substances and the like which do not deleteriously react with the active compounds. In some embodiments, an anti-LC/A toxin VHH polypeptide or multimeric form thereof, and/or an anti-LC/B toxin VHH polypeptide or multimeric form thereof are combined, where desired, with other active substances, e.g., enzyme inhibitors, to reduce metabolic degradation. An effective amount of a pharmaceutical composition can vary according to choice or type of an anti-LC/A toxin VHH polypeptide or multimeric form thereof, and/or an anti-LC/B toxin VHH polypeptide or multimeric form thereof as described herein, the particular composition formulated, the mode of administration and the age, weight and physical health or overall condition of the patient, for example. In an embodiment, an effective amount of an anti-LC/A toxin VHH polypeptide or multimeric form thereof, and/or an anti-LC/B toxin VHH polypeptide or multimeric form thereof (and/or anti-epitope tag antibody if one or more of the VHH polypeptides or multimeric form thereof contains an epitope tag) is an amount which is capable of reducing one or more symptoms of disease or pathology caused by a botulinum bacterial infection and the production of its toxins, namely, the LC/A and LC/B neurotoxins.

In certain embodiments, a composition includes one or more polynucleotide sequences that encode one or more anti-LC/A toxin VHH polypeptides, and/or anti-LC/B toxin VHH polypeptides or multimeric forms thereof as described herein. In an embodiment, a polynucleotide sequence encoding an anti-LC/A toxin VHH polypeptide or multimeric form thereof, and/or an anti-LC/B toxin VHH polypeptide or multimeric form thereof is in the form of a DNA molecule or multimer. In some embodiments, the composition includes a plurality of nucleotide sequences each encoding an anti-LC/A toxin VHH polypeptide or multimeric form thereof, and/or an anti-LC/B toxin VHH polypeptide or multimeric form thereof, or any combination of anti-LC/A toxin VHH polypeptides or anti-LC/B toxin VHH polypeptides or multimeric forms thereof as described herein, such that the anti-LC/A toxin VHH polypeptides and/or the anti-LC/B toxin VHH polypeptides or multimeric forms thereof are expressed and produced in situ. In such compositions, a polynucleotide sequence is administered using any of a variety of delivery systems known to those of ordinary skill in the art, including eukaryotic, bacterial, viral vector nucleic acid expression systems.

Suitable nucleic acid expression systems contain appropriate nucleotide sequences operably linked for expression in a patient (such as suitable promoter and termination signals). In an embodiment, a polynucleotide molecule encoding an anti-LC/A toxin VHH polypeptide or multimeric form thereof, and/or an anti-LC/B toxin VHH polypeptide or multimeric form thereof can be introduced using a viral expression system (e.g., vaccinia or other pox virus, retrovirus, lentivirus, or adenovirus associated virus (AAV)), which uses a non-pathogenic (defective), replication competent virus. Techniques for incorporating nucleic acid (DNA) into such expression systems are well known to and practiced by those of ordinary skill in the art. The nucleic acid (DNA) can also be “naked,” as described, for example, in Ulmer et al., 1993, Science, 259:1745-1749 and as reviewed by Cohen, 1993, Science 259:1691-1692. Polynucleotides encoding the anti-LC/A or LC/B toxin VHHs may be delivered to cells or administered to a subject through the use of nanoparticles, e.g., lipid nanoparticles, comprising DNA or RNA, or coating the DNA or RNA onto biodegradable beads.

### Methods of Detection

Also encompassed are methods of detecting botulinum toxins in a sample using the LC/A toxin-binding and/or LC/B toxin-binding VHH polypeptides described herein. The sample may be a biological sample obtained from a subject, e.g., blood, plasma, serum, saliva, sputum, tears, mucus, urine, stool, cell or tissue, and the like, or a sample obtained from the environment or a natural source, e.g., soil, water, or from a substrate or surface of any object or material capable of being tested. The VHH polypeptides may be coupled to or labeled with a detectable molecule or moiety, as known by those in the art, and detection and quantification of toxin protein can be accomplished using any number of known detection methods and technologies, e.g., immunoassays, sandwich immunoassays including enzyme linked immunosorbent assays (ELISA), fluorescence-based, luminescent-based, chemiluminescent-based, or electrochemical assays and immunoassays, immunoblots, Western Blots, as well as other enzyme immunoassays. By way of example, detection of botulinum LC/A and/or LC/B toxin (and/or levels thereof) are determined by contacting the sample with one or more of the VHH polypeptides (antibodies), or toxin-binding fragments thereof, that selectively bind to toxin protein (LC/A and/or LC/B); and detecting binding of the VHH polypeptides (antibodies), or toxin-binding fragments thereof, to toxin protein, if present in the sample. The detectable label or moiety allows detection of binding complexes, or toxin bound to the VHH polypeptides.

By way of nonlimiting example, the assay performed on the sample, e.g., a biological or natural source sample, involves contacting the sample with one or more anti-LC/A and/or LC/B VHH antibodies (polypeptides), which may be detectably labeled, to form a toxin protein and VHH antibody complex. The complexes can then be detected and/or quantified; levels of the protein bound can be compared to the binding of one or more positive (e.g., known toxin protein or synthetic form thereof) or negative (e.g., an irrelevant, non-LC/A or LC/B protein) reference controls or standards. In an embodiment, one or a panel or plurality of the anti-LC/A- and/or LC/B-binding VHH antibodies can be immobilized on a suitable solid-phase substrate, support, or carrier. The test sample is contacted with the immobilized VHH antibodies and incubated for a desired period of time. After washing to remove unbound material, detection of binding of protein in the sample to the VHH antibodies is performed. In an embodiment, a detectable secondary antibody that binds to the VHH antibodies can be used for detecting of binding of VHH polypeptides to the protein in the sample. The secondary detection antibody may be conjugated, either directly or indirectly, to a detectable moiety. Nonlimiting examples of detectable moieties that can be employed in the methods include chemiluminescent and luminescent agents; fluorophores such as fluorescein, rhodamine and eosin; radioisotopes; colorimetric agents; and enzyme-substrate labels, such as biotin-avidin.

Solid-phase substrates, supports, or carriers for use in the methods are well known to those in the art and include, for example, multi-well microtiter plates, glass, paper, and microporous membranes constructed, for example, of nitrocellulose, nylon, polyvinylidene difluoride, polyester, cellulose acetate, mixed cellulose esters and polycarbonate, and the like. Example of suitable microporous membranes include, for example, those described in U.S. Pat. No. 9,625,453. Methods for the automation of immunoassays are well known in the art and include, for example, those described in U.S. Pat. Nos. 5,885,530, 4,981,785, 6,159,750 and 5,358,691. The use of a multiplex ELISA detection method, which offers the advantages of high throughput, a small volume of sample being required, and the ability to detect proteins and different proteins across a board dynamic range of concentrations, is also envisioned. Multiplex arrays in different formats based on, for example, flow cytometry, chemiluminescence or electron-chemiluminescence technology, are well known in the art. Flow cytometric multiplex arrays, also known as bead-based multiplex arrays, include the Cytometric Bead Array (CBA), (BD Biosciences, Bedford, MA), and multi-analyte profiling (xMAP®) technology (Luminex Corp., Austin, TX), which employ bead sets that are distinguishable by flow cytometry, as each bead set is coated with a specific binding (or capture) antibody. Fluorescence or streptavidin-labeled detection antibodies bind to the specific capture antibody-target protein complexes formed on the bead set. Multiple biomarkers can be recognized and measured by differences in the bead sets, with chromogenic or fluorogenic emissions being detected using flow cytometric analysis. In an alternative exemplary format, a multiplex ELISA (Quansys Biosciences, Logan, UT) involves the use of multiple, specific capture antibodies coated onto the same well of a multi-well microtiter tissue culture plate at multiple spots (one antibody at one spot). Chemiluminescence technology is used to detect multiple proteins at the corresponding spots on the plate. Numerous other assay formats that utilize antibodies, e.g., VHH antibodies, bound to a substrate, or in solution, and which specifically bind to and capture an analyte or target protein, polypeptide, or peptide that may be present in a test sample, as known and practiced in the art, are encompassed for use with the VHH antibodies described herein.

### Kits

Provided herein are kits for use in connection with the anti-LC/A toxin VHH polypeptide and anti-LC/B toxin VHH polypeptide products, compositions and methods described herein. In an aspect, the kits are used in connection with treatments (therapeutic treatments) to inhibit botulinum toxin activity in an intoxicated patient having botulism. In some embodiments, the kit includes an effective amount of anti-LC/A toxin VHH polypeptide and/or an anti-LC/B toxin VHH polypeptide, or a multimer thereof, as described herein, in unit dosage form. In other embodiments, the kit includes a therapeutic or prophylactic composition containing an effective amount of an anti-LC/A toxin VHH polypeptide and/or an anti-LC/B toxin VHH polypeptide or multimer thereof in unit dosage form. In still other embodiments, the kit includes a therapeutic or prophylactic composition containing an effective amount of one or more of an anti-LC/A toxin VHH polypeptide and/or an anti-LC/B toxin VHH polypeptide or multimer form thereof, and an anti-epitope tag antibody, in unit dosage form. In some embodiments, the kit comprises a device (e.g., an automated or implantable device for subcutaneous delivery; an implantable drug-eluting device, or a nebulizer or metered-dose inhaler) for dispersal of the composition or a sterile container which contains a pharmaceutical composition. Non-limiting examples of containers include boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, a pharmaceutical composition of the invention is provided together with instructions for administering the pharmaceutical composition containing an anti-LC/A toxin VHH and/or an anti-LC/B toxin polypeptide to a subject having, or at risk of contracting or developing, disease or pathology, and/or the symptoms thereof, associated with infection by botulinum bacteria (e.g., C. botulinum). The instructions will generally include information about the use of the composition for the treatment or prevention of intoxication by the bacteria and the neurotoxin proteins (LC/A toxin or LC/B toxin) that they produce. In other embodiments, the instructions include at least one of the following: description of the therapeutic/prophylactic agent; dosage schedule and administration for treatment or prevention of disease or symptoms thereof caused by one or more of the disease-causing bacteria and/or the botulinum toxin proteins that they produce; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

### Example 1: Discovery and Characterization of VHHs Targeting the LC of BoNT/A and BoNT/B

VHHs targeting BoNT/A and BoNT/B [6, 8], some of which recognize the light chain protease domains of these toxins (LC/A and LC/B), have been reported. VHHs have also been identified specifically for their binding to LC/A [11,12]. These VHHs were typically selected based on their ability to bind to the LC in the context of holotoxins or the isolated LC, which were immobilized by coating to plastic plates. Recent information, particularly with regard to the LC of BoNT/E (LC/E), has demonstrated that BoNT LC can become conformationally altered when coated to plastic [7]. VHHs are known to be highly dependent on the 3D conformation of their respective epitopes for binding [13]. Accordingly, many useful VHHs are likely to have been missed from earlier efforts to select VHHs for their affinity to plastic-coated BoNT proteases. New discovery and characterization efforts were performed to identify VHHs that bound to BoNT LC/A and LC/B in which protease targets that were antibody-captured were employed so as to retain their native conformational state in solution.

To discover new VHHs to LC/A, a phage-displayed library was prepared from two alpacas that had been hyperimmunized with LC/A. VHHs were selected that bound to LC/A immobilized by capture to ALc-B8 VHH [12]. ALc-B8 binds to soluble LC/A with high affinity and inhibits its protease activity. The VHH capture panning resulted in the discovery of 13 new unique LC/A-binding VHHs that had not been previously identified by panning on plastic-coated LC/A [12]. The sequences of these new VHHs (JPU series) are shown in FIG. 8. Also included for comparison are sequences of several previously identified LC/A-binding VHHs that were reported elsewhere (ciA-D1, ciA-D12, ciA-F12, ciA-H7, ALc-H7) [6, 12]. The below Table 1 sets forth the new LC/A-binding VHHs described herein, as well as the CDR1-3 sequences, which are also shown in FIG. 8.

TABLE 1
VHH polypeptides that bind to Botulinum neurotoxin (BoNT) serotype A (LC/A)

| VHH name | CDR1 | CDR2 | CDR3 |
|---|---|---|---|
| JPU-A1 | SGFTLDDYAIGWF | SRSGDTYYP | DFPPVRPMCIQAAPKKR |
| | (SEQ ID NO: 38) | (SEQ ID NO: 39) | (SEQ ID NO: 40) |
| JPU-A5 | SGADFSFYAMGWY | NLNGVISYG | MRLYTRGSVRHPESW |
| | (SEQ ID NO: 41) | (SEQ ID NO: 42) | (SEQ ID NO: 43) |
| JPU-A11 | TGRTLDYYALGWF | NWLGGSTYYA | DFSIAYSGTYPPAYAEYDYDYW |
| | (SEQ ID NO: 44) | (SEQ ID NO: 45) | (SEQ ID NO: 46) |
| JPU-B5 | SGSPLSIWVMGWY | NLNGITSYG | EPLGPRGKKSGKEYW |
| | (SEQ ID NO: 47) | (SEQ ID NO: 48) | (SEQ ID NO: 49) |
| JPU-B9 | TSENVFGIYGMAW | ITSRGTAHYH | GPYW |
| | (SEQ ID NO: 50) | (SEQ ID NO: 51) | (SEQ ID NO: 52) |
| JPU-C1 | SGFTFNRYVIRWY | SRSGDSGRYV | LNLEDMEYW |
| | (SEQ ID NO: 53) | (SEQ ID NO: 54) | (SEQ ID NO: 55) |
| JPU-C10 | SGNIFSIYYMGWY | NSNGITNYG | GKLRRTTGWGLDDYW |
| | (SEQ ID NO: 56) | (SEQ ID NO: 57) | (SEQ ID NO: 58) |
| JPU-D12 | SGFTLDEYAIGWF | SSSASISYA | AFLACGPVAGWGTEYDYW |
| | (SEQ ID NO: 59) | (SEQ ID NO: 60) | (SEQ ID NO: 61) |
| JPU-G3 | STTISDFYSMGWF | RRGGDTKSG | NLQKSSDELGPYYW |
| | (SEQ ID NO: 62) | (SEQ ID NO: 63) | (SEQ ID NO: 64) |
| JPU-G7 | SLLTLEYYAIGWF | GSSGGSTVYI | DDLRCGRGWSSYFRGSW |
| | (SEQ ID NO: 65) | (SEQ ID NO: 66) | (SEQ ID NO: 67) |
| JPU-G11 | SESVFEMYTVAWY | TDEGRTNYA | EHDLGYYDYW |
| | (SEQ ID NO: 68) | (SEQ ID NO: 69) | (SEQ ID NO: 70) |
| JPU-G12 | SGLTLDYYAIGWF | SSGSSMSIHA | DDFTCGSRWSDWAHTFGFW |
| | (SEQ ID NO: 71) | SEQ ID NO: 72) | (SEQ ID NO: 73) |
| JPU-H7 | SGGIFSTYIMGWY | SNHTTDYA | DWMVGAWTAGDYGVDYW |
| | (SEQ ID NO: 74) | SEQ ID NO: 75) | SEQ ID NO: 76) |

A panel of newly identified LC/B-binding VHHs was identified from a phage-display library prepared from two alpacas immunized with purified LC/B and selected for binding to LC/B captured on plastic with VHH BLc-B10 or JND-E4. VHH BLc-B10 was previously identified by panning on plastic-coated LC/B [12], which was used here as a LC/B-capturing agent to discover seven unique new LC/B-binding VCs (JND-series). One of the new VHHs, JND-E4, was subsequently employed as the LCB-capturing VHH in a new round of panning in order to block a dominant epitope, which led to the discovery of an additional seven unique LC/B-binding VHHs (JSG series). The sequences of these 14 VHHs are shown in FIG. 9 aligned with the sequences of BLc-B10 and three other previously reported LC/B-binding VHHs (JLJ-F9, JLJ-G3, JNE-B10) discovered following selection on ciBoNT/B holotoxin [8]. The below Table 2 sets forth the new LC/B-binding VHHs described herein, as well as the CDR1-3 sequences, which are also shown in FIG. 9.

TABLE 2
VHH polypeptides that bind to Botulinum neurotoxin (BoNT) serotype B (LC/B)

| VHH name | CDR1 | CDR2 | CDR3 |
|---|---|---|---|
| JND-A12 | SGLSFNWYDVG | SRSSGGGSTY | AADWTGRAGFSVGYYRPDEYDY |
| | (SEQ ID NO: 77) | SEQ ID NO: 78) | (SEQ ID NO: 79) |
| JND-B4 | SGFTLDSYAIG | CMSSGDGSTY | AADGFDYCSAYVPGRGMNY |
| | (SEQ ID NO: 80) | SEQ ID NO: 81) | (SEQ ID NO: 82) |
| JND-C7 | SGFTLDNYAVG | CISSSDDNTD | AAESPTFGFSCTVATDPYDY |
| | (SEQ ID NO: 83) | SEQ ID NO: 84) | SEQ ID NO: 85) |
| JND-E4 | SGFTLDGYAAG | WISSTDGSTY | TAGLGLDVSDYVYDY |
| | (SEQ ID NO: 86) | (SEQ ID NO: 87) | SEQ ID NO: 88) |
| JND-E5 | SGFTLDYYGIG | CITSGGLTN | AIDRVGVCAMEDFGS |
| | (SEQ ID NO: 89) | (SEQ ID NO: 90) | SEQ ID NO: 91) |
| JND-E9 | SGRTFNYYAMA | FINWSGDSTY | AAEFGTFSYLQGDDYSY |
| | (SEQ ID NO: 92) | (SEQ ID NO: 93) | SEQ ID NO: 94) |
| JND-F3 | SGRSFSSYRMG | GISWSGSSTW | AADGLGTDWSDAIWDY |
| | (SEQ ID NO: 95) | (SEQ ID NO: 96) | SEQ ID NO: 97) |
| JSG-B8 | SGRMFNEYRMG | AINWGAQIPY | AADWGYGSSPHQDKEYDY |
| | (SEQ ID NO: 98) | (SEQ ID NO: 99) | (SEQ ID NO: 100) |
| JSG-B10 | SGRTFSDYAMG | AVDWSGSSRL | AAARNRWSSEISSYDY |
| | (SEQ ID NO: 101) | SEQ ID NO: 102) | (SEQ ID NO: 103) |
| JSG-C1 | SGRTFRRNTMG | AISWSGDRTY | AADGTASVENSYASADRNKYNY |
| | SEQ ID NO: 104 | (SEQ ID NO: 105) | (SEQ ID NO: 106) |
| JSG-F6 | SIRTFSTSTTA | RISGSDPVTY | ATVRIKGGSEFSYHY |
| | (SEQ ID NO: 107) | (SEQ ID NO: 108) | (SEQ ID NO: 109) |
| JSG-G1 | SESTFSINAIG | HISTSGRTR | NAEGYSTWPEDRYLEL |
| | (SEQ ID NO: 110) | (SEQ ID NO: 111) | (SEQ ID NO: 112) |
| JSG-G10 | SGRTFSSYRMGMG | TVNWSGGTTY | AAGRGSESYTSSRYNY |
| | (SEQ ID NO: 113) | (SEQ ID NO: 114) | (SEQ ID NO: 115) |
| JSG-G11 | SGFTLDDYAIG | CISIRDGRTH | AAGQRSMAYVCSNRFGS |
| | (SEQ ID NO: 116) | (SEQ ID NO: 117) | (SEQ ID NO: 118) |

Provided herein are LC/A toxin-binding VHH polypeptides of SEQ ID NOs: 7-19 as set forth in FIG. 8, or polynucleotides encoding the polypeptides, as well as LC/B toxin-binding VHH polypeptides of SEQ ID NOs: 24-37 as set forth in FIG. 9, or polynucleotides encoding the polypeptides. Encompassed herein are LC/A toxin-binding or LC/B toxin-binding polypeptides having amino acid sequences that have least 85%, at least 90%, at least 93%, at least 95%, at least 97%, at least 98%, at least 99%, or greater, (and percentages therebetween) identity to one or more of the LC/A toxin-binding VHH polypeptides of SEQ ID NOs: 7-19 or the LC/B toxin-binding VHH polypeptides as described herein, or polynucleotides encoding the polypeptides.

The amino acid sequences of representative LC/A toxin binding VHH polypeptides or LC/B toxin-binding VHH polypeptides are shown below in Table 1A and in FIG. 8, and in Table 2A and in FIG. 9, respectively.

TABLE 1A
VHH Polypeptides that bind LC/A botulinum toxin

| VHH | |
|---|---|
| Name | |
| BLc-B8 | SGGGLVQPGGSLRLSCAASGSIFSIYAMGWYRQAPGKQRELVAAISS-YGSTNYADSVKGRFTISRDNAKNTVYLQMNSLKPEDTAVYYCNA--------DIATMTAVGGFDYWGQGTQVTVSS |
| | (SEQ ID NO: 1) |
| ALc-H7 | SGGGSVQPGGSLRLSCAAIGSVFTMYTTAWYRQTPGNLRELVASITD-EHRTNYAASAEGRFTISRDNAKHTVDLQMTNLKPEDTAVYYCKL------------EHDLGYYDYWGQGTQVTVSS |
| | (SEQ ID NO: 2) |
| ciA-D1 | SGGGLVQPGGSLRLSCATSGFTLEYYAIGWFRQAPGKGREGVACMNSSGGGTNYADSVKGRFTISRDNAKKMVYLQMNSLKSEDTAVYYCVV-----DDFRCGSRWAAYLRSSWGQGTQVTVSS |
| | (SEQ ID NO: 3) |
| ciA-D12 | SGGGLVQPGGSLRLSCVVSGSDFNTYIMGWYRQVFGKPRELVADITT-EGKTNYGGSVKGRFTISRDNAKNTVYLQMFGLKPEDAGNYVCNA-----DWKMGAWTAGDYGIDYWGKGTLVTVSS |
| | (SEQ ID NO: 4) |
| ciA-F12 | SGGGLVQPGGSLRLSCAASGFTLGSRYMSWVRQAPGEGFEWVSSIEP-SGTAWDGDSAKGRFTTSRDDAKNTLYLQMSNLQPEDTGVYYCAT---------GYRTDTRIPGGSWGQGTQVTVSS |
| | (SEQ ID NO: 5) |
| ciA-H7 | SGGGLVQVGGSLRLSCVVSGSDISGIAMGWYRQAPGKRREMVADIFS-GGSTDYAGSVKGRFTISRDNAKKTSYLQMNNVKPEDTGVYYCRL---------------YGSGDYWGQGTQVTVSS |
| | (SEQ ID NO: 6) |
| JPU-A1 | SGGGLVQPGGSLRLSCAASGFTLDDYAIGWFRQVPGKEDEGVSCMSR-SGDTYYPHSVKGRFTISVDNAKNTMYLQMNNEKPEDTAVYYCAI-----DFPPVRPMCIQAAPKKRSRGTQVTVSA |
| | (SEQ ID NO: 7) |
| JPU-A5 | TGGGLVQAGGSLRLSCTASGADFSFYAMGWYRQTPGNSRELVAVMNL-NGVISYGDSARGRFDISRDGTKNIVFLQMNSLKPEDTGVYYCNG-------MRLYTRGSVRHPESWGQGIQVTVSS |
| | (SEQ ID NO: 8) |
| JPU-A11 | TGGGLVQAGDSLTLSCAATGRTLDYYALGWFRQVPGNKREFVAAINWLGGSTYYADSVRGRFTLSRDNSKSTLYLNMNNLIPDDTAVYYCAADFSIAYSGTYPPAYAEYDYDYWGQGTQVTVSS |
| | (SEQ ID NO: 9) |
| JPU-B5 | S-GGLVQPGGSLKLSCAHSGSPLSIWVMGWYRQAPGKQRELVALINL-NGITSYGDSVKGRFTISRDYAENTAYLQMNSLKFEDTAVYYCNA-------EPLGPRGKKSGKEYWGTGTQVTVSL |
| | (SEQ ID NO: 10) |
| JPU-B9 | T-GGALVQPGQSLTLSCTTSENVFGIYGMAWLRQAPGRQRELVASITSRGTAHYHDSVKGRFTISRESGKTTAYLQTTSVNPEDTAIYYCNS------------------GPYWGQGTQVTVSS |
| | (SEQ ID NO: 11) |
| JPU-C1 | SGGGLVQPGGSLRLSCAASGFTFNRYVIRWYRQAPGKERELVAGISRSGDSGRYVDSVKGRFTISRDNDKNMAYLQMSSLKPDDTAVYYCSA-------------LNLEDMEYWGQGTQVTVSS |
| | (SEQ ID NO: 12) |
| JPU-C10 | SGGGLVQPGGSLRLSCAASGNIFSIYYMGWYRQAPGKQREMVAIINS-NGITNYGDFVKGRFTISRDNAENSAYLQMNNLTPEDTAVYYCNA-------GKLRRTTGWGLDDYWGQGTQVTVSS |
| | (SEQ ID NO: 13) |
| JPU-D12 | SGGGTVQPGGTLRLSCAASGFTLDEYAIGWFRQAPGKEREGVSCISS-SASISYADSVKGRFTISRDNAKNTVYLTMNSLKPEDTGVYYCAR----AFLACGPVAGWGTEYDYWGQGTQVTVSS |
| | (SEQ ID NO: 14) |
| JPU-G3 | TGGGLVQPGGSLRLSCTASTTISDFYSMGWFRQTPGNQRELVAIVRR-GGDTKSGDSVKGRFTISRDNTRSTVYLQMDNLKPEDTAVYYCYA--------NLQKSSDELGPYYWGQGTQVTVSS |
| | (SEQ ID NO: 15) |
| JPU-G7 | SGGGLVQSGGSLRLSCAASLLTLEYYAIGWFRQAPGKEREGVSCTGSSGGSTVYIDSVKGRFTVVRDNAKNMVYLQMDNLQPEDTAVYYCAA-----DDLRCGRGWSSYFRGSWGQETQVTVSS |
| | (SEQ ID NO: 16) |
| JPU-G11 | TGGGLVQPGGSLRLACVASESVFEMYTVAWYRQAPGKQRELVAGITD-EGRTNYADFVKGRFTISRDNSKKTVHLQMDNLNPEDIAVYYCKL------------EHDLGYYDYWGQGTQVTVSS |
| | (SEQ ID NO: 17) |
| JPU-G12 | SGGGLVQPGGSLRLSCAASGLTLDYYAIGWFRQAPGKEREGVSCISSGSSMSIHADSVKGRFTISRDNAKNTVYLQMNSLKPEDTAVYYCAA---DDFTCGSRWSDWAHTFGPWGQGTQVTVSS |
| | (SEQ ID NO: 18) |
| JPU-H7 | S-GGLVQPGGSLTLSCVVSGGIFSTYIMGWYRQVPGRQREMWATISN--HTTDYADFVQGRFTISRDIAKKAVYLQMHSLKPDDTGRYVCNA-----DWMVGAWTAGDYGVDYWGKGILVTVSS |
| | (SEQ ID NO: 19) |

TABLE 2A
VHH Polypeptides that bind LC/B botulinum toxin

| VHH | | SEQ |
|---|---|---|
| Name | | ID NO. |
| BLc-B10 | SGGGMVQPGGSLRLSCAASGFT--FSTYDMSWVRQAPGKGPEWVSIINAGGGSTYYAASVKGRFAISRDNAKNTLYLQMNNLKPEDTALYYCAR--VASYYCRGYVCSPPEFDYWGQ | 20 |
| | GTQVTVSS | |
| JLJ-F9 | SGGGLVQAGGSLRLSCAPSRLT--LDFFAIAWFRQAPGKEREGVSCISSHDGSTYYTDSVKGRFTISKDNAKNTVYLQMNSLKPEDTAVYYCAL----DHNVGTCQLTQAEYDYWGQ | 21 |
| | GTQVTVSS | |
| JLJ-G3 | SGGGLVQSGGSLRLSCAASGSI--DSLYHMGWYRQAPGKERELVARVQD-GGSTAYKDSVKGRFTISRDFSRSTMYLQMNSLKPEDTAIYYCAA-----------KSTISTPLSWGQ | 22 |
| | GTQVTVSS | |
| JNE-B10 | SGGGLVQPGGSLRLSCAASGFP--FHAYYMSWVRQAPGKGLEWVSHIGNGGIITRYADSVKGRFTISRDNAKNTLYLQMTNLKPEDTALYYCTL-----------GTRDDLGPERFQ | 23 |
| | GTQVTVSS | |
| JND-A12 | TGGGLVQAGGSLGLSCAASGLS--FNWYDVGWFRQAPGKEREFVASRSSGGGSTYYGDSVKGRFSISTDNAKNTAYLQMNSLKPEDTAVYYCAADWTGRAGFSVGYYRPDEYDYWGQ | 24 |
| | GTQVTVSE | |
| JND-B4 | TGGGLVQAPGGSLLSCVASGFT--LDSYAIGWFRQAPGKEREGVSCMSSGDFSTYYTNSVKGRFTISRDNAQNTVYLQMNSLKPEDTAVYYCAA---DGFDYCSAYVPGRGMNYSGK | 25 |
| | GTLVTVSS | |
| JND-C7 | TGGGLVQPGGSLRLSCAGSGFT--LDNYAVGWFRQAPGKEREGVSCISSSDDNTDYSDSVKGRFTISRDNAKDTVYLQMNSLKPEDTAIYYCAA--ESPTFGFSCTVATDPYDYWGQG | 26 |
| | TQVTVSS | |
| JND-E4 | TGGGLVQPGGSLRLSCAASGFT--LDGYAAGWFRQAPGKERELVSWISSTDGSTYYAASVKGRFTVSRDNAKNTVYLQMNSLKPEDTAVYYCTA-------GLGLDVSDYVYDYWGQG | 27 |
| | TQVTVSS | |
| JND-E5 | S-GGLVQPGGSLRLSCAASGFT--LDYYGIGWVRQAPGKEREEVSCITS-GGLTNYPDSVKGRFTISRDNAKNTVYLQMNSLKPEDTAVYYCAI-------DRVGVCAMEDFGSWGQG | 28 |
| | TQVTVSS | |
| JND-E9 | TGGGLVQAGDSLRLSCAASGRT--FNYYAMAWFRQAPGKEREFVAFINWSGDSTYYAGSVKGRFTISRDNAKNTVYLQMNNLKPEDTAVYSCAA-----EFGTFSYLQGDDYSYWGQG | 29 |
| | TQVTVSS | |
| JND-F3 | SGGGLVQAGGSLRLSCAASGRS--FSSYRMGWFRQAPGKERELVAGISWSGSSTWYADSVKGRFTISRDNAKNTVYLQMNSLKPEDTAVYYCAA------DGLGTDWSDAIWDYWGQG | 30 |
| | TQVTVSS | |
| JSG-B8 | SGGGLVQAGGSLRLSCAVSGRM--FNEYRMGWFRQAPGKEREFVSAINWGAQIPYYADSVKGRFTISRDSAENTVYLQMNSLKPEDTAVYYCAA----DWGYGSSPHQDKEYDYWGQG | 31 |
| | TQVTVSS | |
| JSG-B10 | SGGGLVQAGGSLRLSCAASGRT--FSDYAMGWFRQAPGKERVFVAAVDWSGSSRLYAESVEGRFTISRDNSKNTVYLQMNTLKPEDTAVYYCAA------ARNRWSSEISSYDYWGQG | 32 |
| | TQVTVSS | |
| JSG-C1 | SGGGLVQTGGSLRLSCAASGRT--FRRNTMGWFRQAPGKVREFVAAISWSGDRTYCADSVKGRFTISRDNAKNTVDLLMNSLKPEDTAIYYCAADGTASVFNSYASADRNKYNYWGQG | 33 |
| | TQVTVSS | |
| JSG-F6 | SGGGLVQAGDSLRLSCAASIRT--FSTSTTAWFRQAPGKEREFVARISGSDPVTYYTDSVRGRFTISRDNAKSTAYLQMNSLKPEDTGVYYCAT-------VRIKGGSEFSYHYWGQG | 34 |
| | TQVTVSS | |
| JSG-G1 | TGGGLVQAGGSLRLSCAASEST--FSINAIGWYRQAPGKQRELVAHIST-SGRTRYADSVKGRFTISRDNAWNTVFLQMISLKPEDTAVYYCNA------EGYSTWPEDRYLELWGQG | 35 |
| | TQVTVSP | |
| JSG-G10 | TGGGLVQAGGSLRLSCTASGRTFSSYRMGMGWGRQAPGKEREFVATVNWSGGTTYYADSVKGRFTISRDNAKNTVYLQMNSLKPEDTAVYYCAA------GRGSESYTSSRYNYWGQG | 36 |
| | TQVTVSS | |
| JSG-G11 | SGGGLVQPGGSLRLSCAASGFT--LDDYAIGWFRQAPGKEREAVSCISIRDGRTHYADSVKGRFTISRDNAKNTVYLQMNSLKPEDTAVYYCAA-----GQRSMAYVCSNRFGSWGQG | 37 |
| | TQVTVSS | |

The newly identified LC/A- and LC/B-binding VHHs as described herein were characterized for their target binding properties and their potency to inhibit the target proteases (Tables 3 and 4 below). They were first tested for the apparent binding affinities to the appropriate BoNT holotoxin (ciBoNT/A or ciBoNT/B) and isolated protease domain (LC/A or LC/B). Dilution ELISAs to the LCs were performed both by both coating the targets onto plastic or by immobilizing the soluble protease with a capture antibody. As found with the LC/E-binding VHHs [7], binding affinity was often substantially compromised by coating the proteases onto plastic. Even using the less denaturing tissue culture plastic to coat LC/A or LC/B, the binding was significantly reduced compared to captured LCs for many of the VHHs. Most of the VHHs displayed sub-nM apparent affinities to the captured LCs. Interestingly, the majority of new VHHs selected on the isolated LCs showed poor or undetectable binding to the holotoxins, as a consequence of the unique structure of BoNT holotoxin, where a portion of the heavy chain wraps around the LC like a belt that is believed to acts as a substrate surrogate [14].

TABLE 3
A summary of anti-BoNT/A VHH characterization data

| | ciBoNT/A- | LC/A1- | LC/A1- | | |
|---|---|---|---|---|---|
| | binding | binding | binding | LC/A1 | Compe- |
| VHH | EC₅₀, nM | EC₅₀, nM | EC₅₀, nM | protease | tition |
| name^{A} | coated^{B} | coated^{C} | captured^{D} | inhibition^{E} | group^{F} |
| ALc-B8 | NB | 3 | 0.5 | ++ | 1 |
| ALc-H7 | Trace | 5 | 0.5 | ++ | 1 |
| ciA-D1 | 3 | 1 | 0.4 | − | 2 |
| ciA-D12 | 3 | 1 | 0.2 | − | 3 |
| ciA-F12 | 0.2 | 1 | 0.2 | − | 4 |
| ciA-H7 | 0.2 | 3 | 0.1 | − | 2 |
| JPU-A1 | 1 | 10 | 0.4 | − | 2 |
| JPU-A5 | Trace | 10 | 0.1 | ++++ | 5 |
| JPU-A11 | Trace | 10 | 0.4 | ++ | 6 |
| JPU-B5 | NB | Trace | 0.8 | − | 5 |
| JPU-B9 | NB | NB | 0.8 | + | 5 |
| JPU-C1 | NB | 20 | 0.2 | ++++ | 2 |
| JPU-C10 | NB | Trace | 0.2 | ++++ | 5 |
| JPU-D12 | NB | 5 | 0.2 | +++ | 6 |
| JPU-G3 | NB | 5 | 0.2 | +++ | 2 |
| JPU-G7 | 15 | 10 | 3 | − | 2 |
| JPU-G11 | Trace | 1 | 0.5 | +++ | 1 |
| JPU-G12 | 10 | 10 | 0.5 | − | 2 |
| JPU-H7 | 3 | 3 | 0.8 | − | 3 |

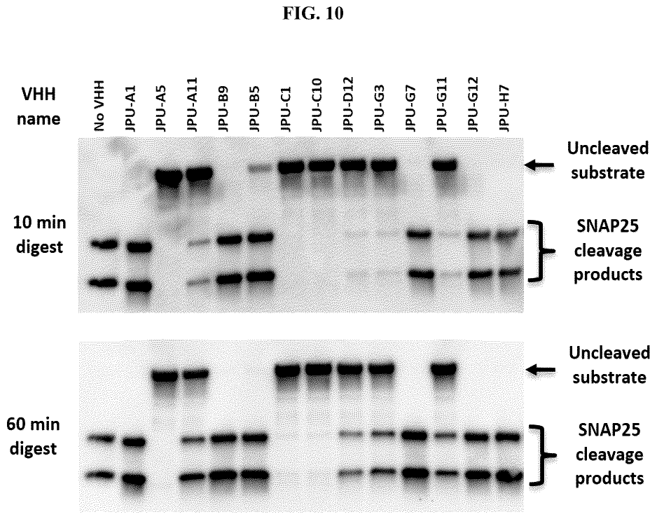

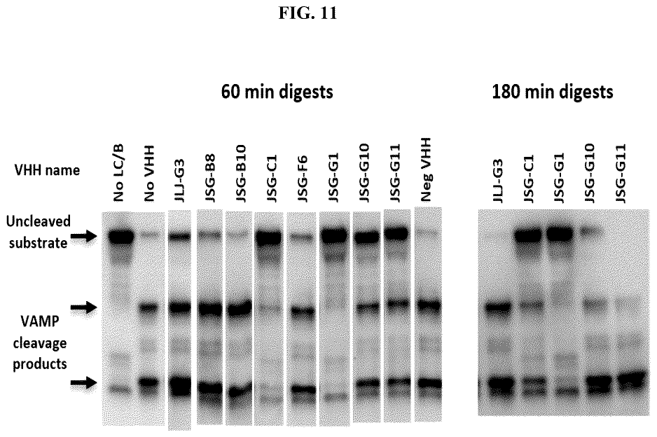

^{A}Lab code given to each VHH
^{B}The EC₅₀ was the VHH concentration that produced about 50% of the peak signal on a serial dilution_{ELISA} on ciBoNT/A-coated Costar plates
^{C}The EC₅₀ was the VHH concentration that produced about 50% of the peak signal on a serial dilution_{ELISA} on LC/A-coated Costar plates
^{D}The EC₅₀ was the VHH concentration that produced about 50% of the peak signal on a serial dilution_{ELISA} on LC/A captured by a LC/A-binding VHH (value shown is the lowest found after testing with several different capture VHHs)
^{E}Based on protease inhibition assays such as shown in FIG. 11
^{F}Based on competition analysis as described in Materials and Methods
NB = no significant binding
Trace = some signal; too low for EC₅₀ assessment (i.e. EC₅₀ >125 nM)
− Showed no inhibition on SNAP25-cleavage assays (e.g. FIG. 10)
+ Showed weak inhibition on SNAP25-cleavage assays (e.g. FIG. 10)
++ Showed moderate inhibition on SNAP25-cleavage assays (e.g. FIG. 10)
+++ Showed strong inhibition on SNAP25-cleavage assays (e.g. FIG. 10)
++++ Showed strong and persistent inhibition on SNAP25-cleavage assays (e.g. FIG. 10)

TABLE 4
A summary of anti-BoNT/B VHH characterization data

| | ciBoNTB- | LC/B1- | LC/B1- | | |
|---|---|---|---|---|---|
| | binding | binding | binding | LC/B1 | |
| VHH | EC₅₀, nM | EC₅₀, nM | EC₅₀, nM | protease | Competition |
| name^{A} | captured^{B} | coated^{C} | captured^{D} | inhibition^{E} | group^{F} |
| BLc-B10 | Trace | 5* | 10 | − | 1 |
| JLJ-F9 | 0.5 | 5 | 0.8 | +/− | 2 |
| JLJ-G3 | 0.1 | 10 | 3 | + | 3^{#} |
| JLO-G7 | 0.1 | 0.5 | 0.5 | +/− | 2 |
| JND-A12 | 15 | 2 | 0.5 | − | 2 |
| JND-B4 | 0.1 | 0.8 | 0.5 | +/− | 2 |
| JND-C7 | Trace | 3 | 0.8 | +/− | 4^{#} |
| JND-E4 | Trace | 1 | 0.3 | − | 4^{#} |
| JND-E5 | Trace | 5 | 2 | − | 1^{#} |
| JND-E9 | 0.5 | 1 | 0.5 | − | 2 |
| JND-F3 | NB | 10 | 0.8 | − | 4 |
| JNE-B10 | 0.2 | 1 | 0.2 | − | 2 |
| JSG-B8 | 25 | 0.8 | 0.1 | − | 2 |
| JSG-B10 | 15 | 2 | 0.1 | − | 2 |
| JSG-C1 | Trace | 5 | 0.5 | +++ | 5 |
| JSG-F6 | Trace | Trace | 2 | + | 5 |
| JSG-G1 | Trace | 1 | 0.1 | +++ | 5 |
| JSG-G10 | Trace | Trace | 3 | ++ | 5 |
| JSG-G11 | NB | 25 | 0.5 | ++ | 5 |

^{A}Lab code given to each VHH
^{B}The EC₅₀ was the VHH concentration that produced about 50% of the peak signal on a serial dilution_{ELISA} on JEQ-H11-captured ciBoNT/B
^{C}The EC₅₀ was the VHH concentration that produced about 50% of the peak signal on a serial dilution_{ELISA} on LC/B-coated Costar plates
^{D}The EC₅₀ was the VHH concentration that produced about 50% of the peak signal on a serial dilution_{ELISA} on LC/B captured by a LC/B-binding VHH (value shown is the lowest found after testing several different VHHs)
^{E}Based on protease inhibition assays such as shown in FIG. 11 as described in Materials and Methods
^{F}Based on competition analysis as described in Materials and Methods
*Binds with 0.3 nM EC₅₀ to LC/B coated to Nunc plastic
^{#}Partially overlapping epitopes
NB = no significant binding by dilution ELISA at 125 nM
Trace = some signal; too low for EC₅₀ assessment (i.e. EC₅₀ >125 nM)
− Showed no inhibition on VAMP-cleavage assays (e.g. FIG. 11)
+/− Showed weak inhibition only at high concentrations on VAMP-cleavage assays
+ Showed moderate inhibition on VAMP-cleavage assays (e.g. FIG. 11)
++ Showed strong inhibition on VAMP-cleavage assays, not very persistent (e.g. FIG. 11)
+++ Showed strong and persistent inhibition on VAMP-cleavage assays (e.g. FIG. 11)

Therefore, the epitopes of many of these newly identified LC-binding VHHs may be masked by the belt in the context of the holotoxin. Competition ELISAs were performed to provide evidence that two or more VHHs bound to overlapping or nearby epitopes on the LCs. From these ELISAs, VHHs representing six different epitope ‘competition groups’ were found on LC/A and five groups were found on LC/B.

In addition to their binding properties, these VHHs were assessed for their ability to inhibit the targeted protease's activity for their SNAP25 or VAMP2 substrates (Tables 1 and 2; FIGS. 10 and 11). In addition to the previously reported LC/A inhibitor, ALc-B8, several additional VHHs displayed potent activities to inhibit the protease cleavage of SNAP25. Of interest, VHHs that inhibit LC/A were identified in four different competition groups (1, 2, 5,6), indicating that multiple protease-inhibiting sites exist. This finding is substantially extended through structural studies described below. For LC/B, many VHHs were found to inhibit VAMP2 cleavage by the protease, although they were found in only two LC/B-binding competition groups (2 and 5), while all of the most potent and persistent proteases were members of the same competition group 5.

### Example 2: Structural Studies on VHH Binding to LC/A or LC/B

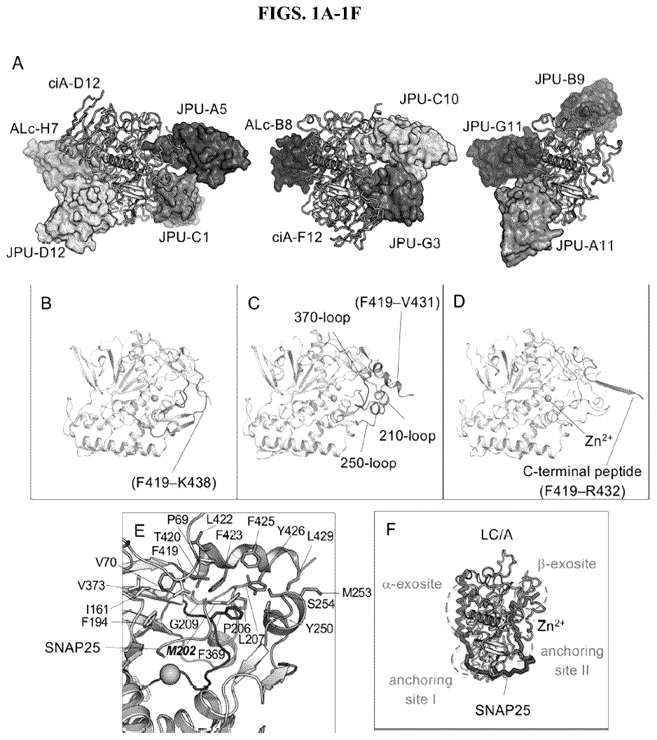

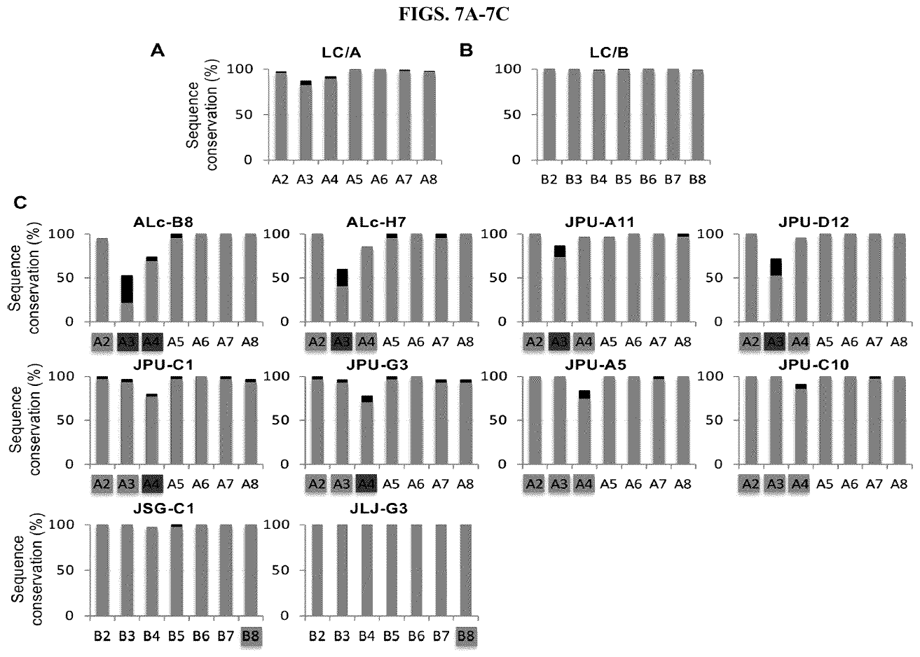

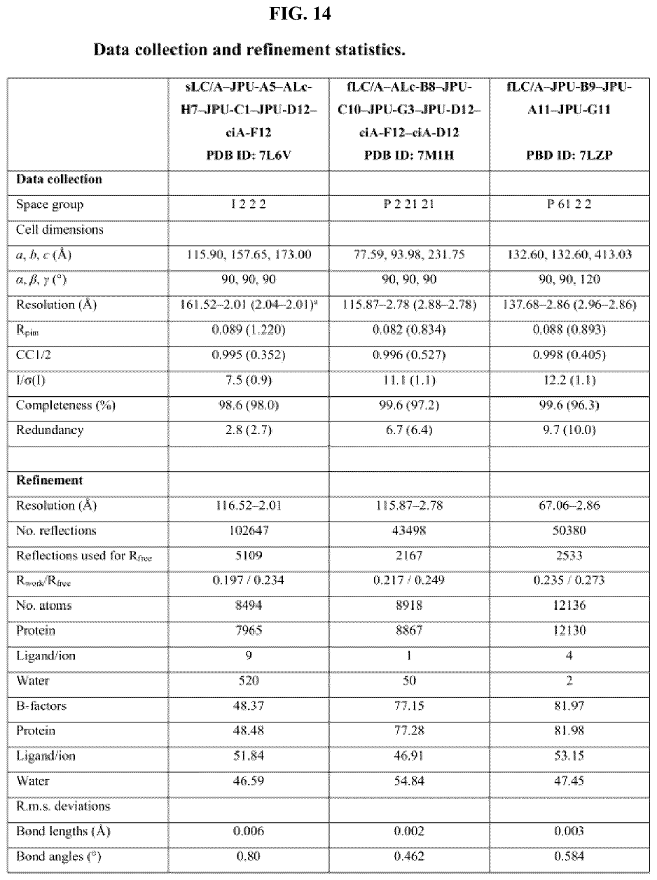

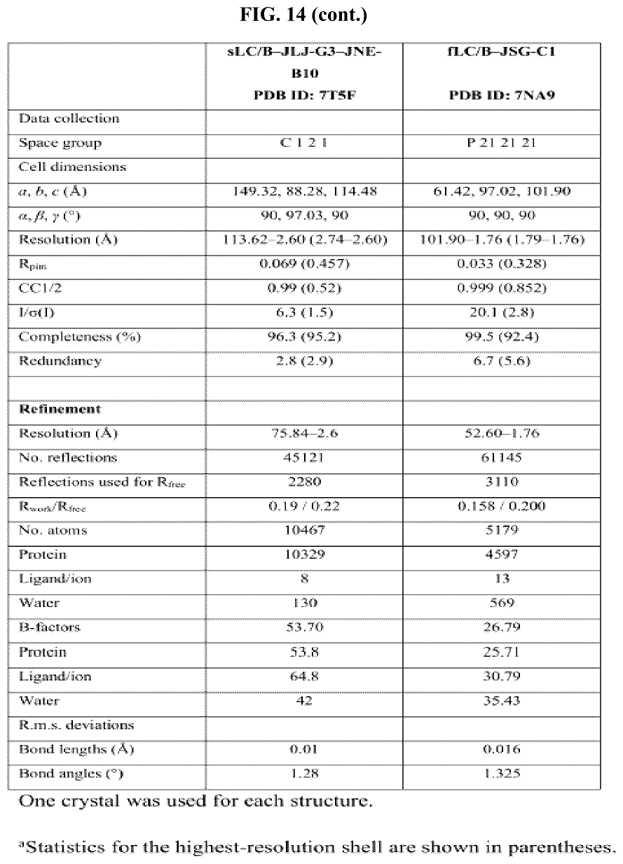

To investigate the inhibition mechanisms of the anti-LC/A and anti-LC/B VHHs, at least two members from each of the LC/A-inhibiting VHH competition groups (groups 1, 2, 5, 6 in Table 3) were selected, along with one member from each LC/B-inhibiting VHH competition group (groups 2, 3, 5 in Table 4) for further study. Inhibitory VHHs within different competition groups must each bind at unique sites associated with protease function. In total, 10 anti-LC/A and 3 anti-LC/B VHHs were selected for structural studies. Either full-length LC (residues 2-438 of LC/A, named fLC/A; 1-441 of LC/B, named fLC/B) or C-terminally truncated LC forms (residues 1-420 of LC/A, named sLC/A; 1-425 of LC/B, named sLC/B) were employed, because truncated BoNT LC has been reported to promote improved protein crystallization [15]. Two of the anti-BoNT/A VHHs, JPU-C10 and JPU-B9, bound only to fLC/A, and hence, fLC/A was used for these two VHHs. Initially, the selected VHHs were individually co-purified with the LC for crystallization. However, this approach resulted in poorly diffracted crystals in some cases (e.g. sLC/A-JPU-A5 diffracted to ˜7 Å). As an alternative, several selected non-competing VHHs were mixed together with the LC for co-crystallization since VHHs are known to promote crystal packing [16]. These included some VHHs that bind to distinct, non-inhibiting LC epitopes such as the previously reported ciA-F12 and ciA-D12 VHHs [6], in order to facilitate crystallization. After extensive screening, the structures of sLC/A in complex with five VHHs including JPU-A5, ALc-H7, JPU-C1, JPU-D12 and ciA-F12; fLC/A in complex with six VHHs including ALc-B8, JPU-C10, JPU-G3, JPU-D12, ciA-F12 and ciA-D12; fLC/A bound to three VHHs: JPU-B9, JPU-A11, and JPU-G11 were determined (FIG. 1A); sLC/B bound to JLJ-G3 and JNE-B10; and fLC/B bound to JSG-C1 (FIG. 7A), all in resolutions ranging from 1.82-2.86 Å (FIG. 14).

### Example 3: Structural Plasticity of the C-Terminus of LC/A is Implicated in its Enzymatic Activity

The full length LC/A, fLC/A, was previously reported to be unsuitable for protein crystallization because of a tendency to aggregate and the natural flexibility of its C-terminal peptide (residues 421-438) [17]. This is why all currently available crystal structures of LC/A are based on truncated LC/A. However, the C-terminus of fLC/A is considered functionally important as it has been reported to play roles in subcellular localization [18], persistence in neurons [19, 20], and catalytic activity [21]. Therefore, it was exciting to identify JPU-C10 or JPU-B9 as VHHs that bind to fLC/A, but not to sLC/A, indicating the direct involvement of the C-terminus of LC/A in VHH binding and possibly in protease activity.

In order to better understand the function of this important C-terminal region on fLC/A, the structures of fLC/A were determined in complex with these two VHHs. Surprisingly, the C-terminal region of fLC/A adopted two different conformations in these two VHH-bound complexes, further supporting the hypothesis that this region possesses natural flexibility in solution (FIGS. 1B and 1C). The two different conformations appear to have been captured as a result of their binding to VHHs. This conformational capture is not surprising as VHHs are widely used for mechanistic studies of the dynamic conformational states of enzymes and membrane proteins [22]. In the JPU-B9-bound fLC/A, the C-terminal peptide forms a loop and a short β-strand, which is similar to the conformation of LC/A observed in the context of holotoxin (FIG. 1D) [23]. A major difference is that the β-strand interacts with the N-terminal region of the HC in the holotoxin while it is stabilized by pairing with the 250-loop in fLC/A. In contrast, the C-terminal peptide of the JPU-C10-bound fLC/A formed an amphipathic α-helix (F419-L429) in which the hydrophobic side was partially buried and facing the active site pocket, while the hydrophilic residues are solvent-exposed (FIG. 1C). The electron density was not observed for residues R432 to K438 in the JPU-C10-bound fLC/A which are solvent-exposed and likely to have high flexibility. Therefore, the C-terminus of LC/A appears to adopt at least two distinct conformations, referred to as the 3-form stabilized by JPU-B9 and the α-form captured by JPU-C10. In both conformations, residue C430 of fLC/A is solvent-exposed and thus likely available to form scrambled disulfide bonds with neighboring molecules in solution. This feature may explain why fLC/Ais prone to aggregation and typically unsuitable for structural studies [24].

Prior functional studies showed that the C-terminal peptide of fLC/A contributes to catalysis of SNAP25 cleavage and substrate binding [18, 21, 24]. Structural analysis of fLC/A reveals two potential roles for the C-terminal peptide in its enzymatic activity. First, the C-terminal helix in the α-form may form a latch that interacts with the 210-, 260-, and 370-loops of LC/A through hydrophobic interactions and thus promotes catalysis by stabilizing the active conformation of LC/A (FIG. 1C). In contrast, the 210-loop and 260-loop in the 3-form are solvent exposed with high local flexibility resembling that of the apo-LC/A (FIGS. 1B and 1D). Second, the C-terminal helix in the α-form, together with the 210-loop and 260-loop, form a hydrophobic pocket which has clusters of aromatic residues (e.g. F423, F425, Y426) that could facilitate the docking of the C-terminal region of SNAP25 to LC/A. In fact, the α-conformation shares a similarity with the SNAP25(197-202)-bound LC/A (PDB code 3DDA) [15, 25] and structural analysis shows that this hydrophobic pocket involving the C-terminal helix will facilitate the binding of the P5′ M202 residue of SNAP25 to the catalytic site (FIG. 1E). Taken together, the experimental data suggest that the C-terminus of fLC/A might dynamically alter between the α- and the β-forms to regulate SNAP25 substrate binding and product release. As such, the structures of fLC/A should be more suitable and informative than sLC/A when performing studies of VHH inhibition mechanisms and for the development of peptide or small molecule inhibitors of LC/A.

### Example 4: VHHs Exploit Diverse Mechanisms to Interact with LC/A and Inhibit its Protease Activity

LC/A recognizes SNAP25 at the catalytic site as well as at multiple distant sites (termed exosites) in a manner such that SNAP25 effectively encircles the protease (FIG. 1F) [15, 25]. Indeed, this unique substrate-binding mode closely resembles the association of LC/A with the heavy chain ‘belt’ region in the context of holotoxin [15]. It is now well established that the N-terminal residues of SNAP25 are docked to a hydrophobic surface of LC/A called the α-exosite, the middle unstructured region of SNAP25 interacts with LC/A through two anchoring points called I and II, and the C-terminus of SNAP25 forms a beta-strand and interacts with the catalytic β-exosite of LC/A (FIG. 1F) [15, 25].

Comparing the structures of SNAP25-bound LC/A with the structures of LC/A in complex with protease-inhibiting VHHs reveals diverse mechanisms by which these VHHs inhibit LC/A cleavage by competing with SNAP25 binding at different sites. Specifically, strong LC/A protease inhibiting VHHs could be classified into four groups: (1) three VHHs bind to the α-exosite of LC/A (ALc-B8, JPU-G11 and ALc-H7); (2) two bind to anchoring site I (JPU-A11 and JPU-D12); (3) two bind to anchoring site II (JPU-C1 and JPU-G3); and (4) two bind to the β-exosite of LC/A (JPU-A5 and JPU-C10). Consistent with competition study results, VHHs in each of these four groups belong to the same competition group and bind to overlapping epitopes (Table 3). Structures were also obtained for three non-inhibitory VHHs in complex with LC/A, and, in each case, as expected, the structures suggest these VHHs should not interfere with SNAP25 binding to the protease. The inhibition mechanisms underlying these four groups of VHHs are described below.

### VHHs Target the β-Exosite of LC/A

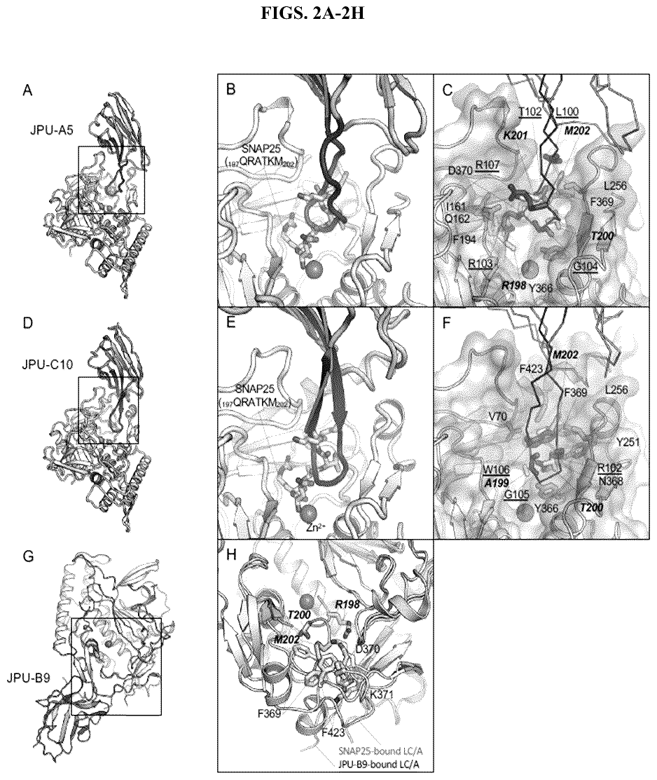

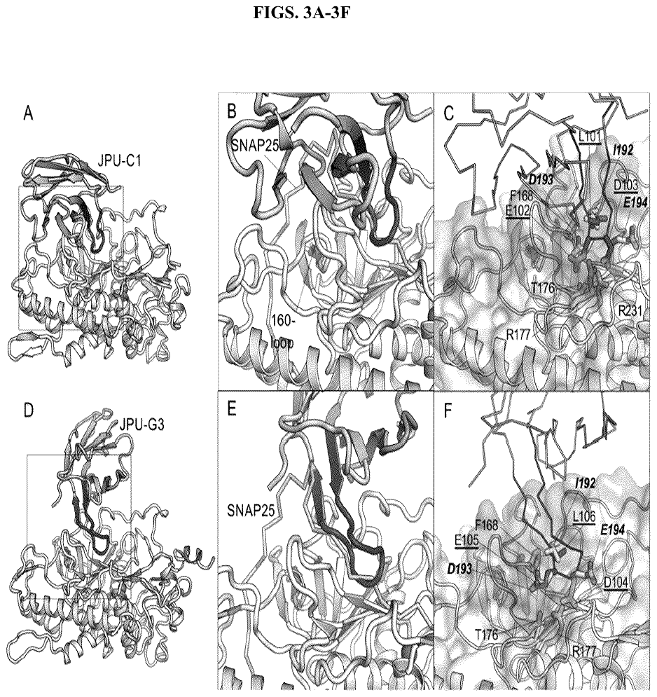

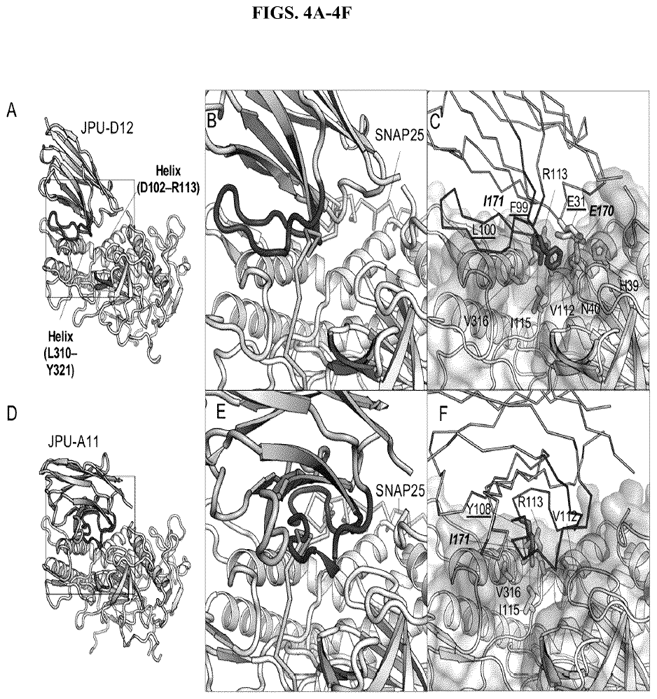

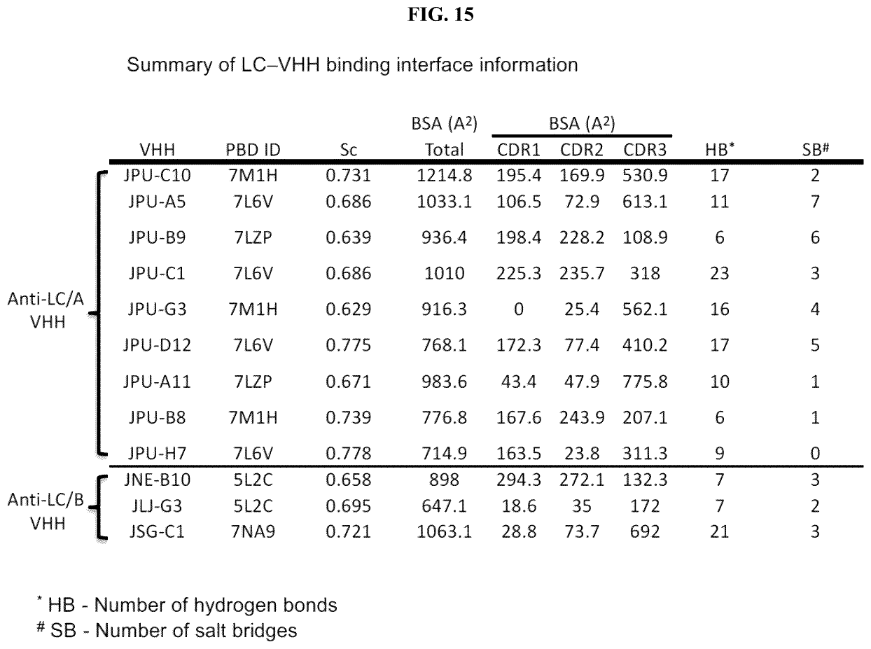

Two potent anti-LC/A VHH inhibitors, JPU-A5 and JPU-C10, bound to the deep active site pocket present at the LC/A β-exosite where the cleavage site and the C-terminus of SNAP25 become associated (FIGS. 2A-2H). JPU-C10 buried a surface area (BSA) of ˜1215 Å² on LC/A (calculated by PDBePISA v1.52) with a surface complementarity (SC) score of 0.731[26], which is higher than the average SC value of ˜0.7 for antibody-antigen complexes [27]. JPU-A5 binding buried a BSA of ˜1033 Å² with an SC score of 0.686 (FIG. 15). Both of the VHHs interact with the 60-, 250-, and 370-loops of LC/A known to be crucial for enzymatic catalysis (FIGS. 2A-2F). The JPU-A5 or JPU-C10-bound LC/A adopted a conformation closely resembling that of LC/A bound to a short peptide of SNAP25 (197-202) (r.m.s.d.=0.49 Å), which represents a pre-cleavage intermediate conformation of LC/A and defines how SNAP25 enters the cleavage site pocket and the S1′-S5′ subsites of LC/A [25]. Interestingly, the CDR3 of both JPU-A5 and JPU-C10 project deeply into the active site of LC/A (FIGS. 2B and 2E). However, since these two VHHs have completely different sequences in their CDR3, they exploit distinct ways to occupy the S1′-S5′ subsites of LC/A to achieve inhibition.

For JPU-A5, its R103 inserts into the S1′ pocket of LC/A by forming a salt-bridge with D370, a hydrogen bond with the carbonyl group of I161, and a cation-π interaction with F194, which is similar to how R198 of SNAP25 engages the S1′ subsite on LC/A (FIG. 2C) [25]. Interestingly several peptide inhibitors of LC/A also exploit the S1′ subsite of LC/A using arginine residues to achieve high affinity binding [28-30]. In addition to R103, G104 and R107 in CDR3 form H-bonds with S3′ and S4′ subsite residues Y366 and Q162, respectively; T102 and L100 in CRD3 interact with the S5′ subsite residues F369 and L256 of LC/A. Furthermore, CDR1 and CDR2 of JPU-A5 also participate in the complex formation by interacting with the 60- and 250-loop of LC/A through hydrogen bonds.

JPU-C10 does not occupy the S1′ subsite of LC/A. Rather, its R102 and G105 interact with S2′ and S3′ subsite residues N368, Y251, Y366 of LC/A (FIG. 2F). Additionally, its W106 forms hydrophobic interaction with the S5′ subsite residues Y251, L256, F369, V70, F423 of LC/A. The CDR3 of JPU-C10 is so close to the cleavage site that its binding should impose steric hindrance that occludes SNAP25 access to the cleavage site pocket. JPU-C10 not only interacts extensively with the S1′-S5′ sites through its CDR3, but it also binds to the C-terminus of LC/A that forms a helix in the complex. In summary, both JPU-A5 and JPU-C10 potently inhibit LC/A activity by blocking SNAP25 interaction with the cleavage pocket of LC/A.

JPU-B9 belongs to the same competition group as JPU-A5 and JPU-C10, but only has a very weak ability to inhibit LC/A protease activity (Table 3; FIG. 10). Structural analysis shows that JPU-B9 binding to LC/A will not directly block the interaction of SNAP25 to the substrate binding pocket. However, JPU-B9 may stabilize the C-terminal loop and the 370-loop of LC/A in a conformation that is unfavorable for substrate binding to the S1′-S5′ subsites (FIGS. 2G and 2H). For example, upon JPU-B9 binding, residue D370 of LC/A, which interacts with the P1′ residue R198 of SNAP25 and is thus critical for catalysis [31], appears to be flipped to the opposite side through hydrogen bonds with residues at the C-terminal loop of LC/A and therefore may not be accessible for SNAP25 binding. Furthermore, the position of D370 in apo-LC/A is occupied by the positively charged K369 in the JPU-B9-bound form, which should inhibit the binding of R198 of SNAP25 due to charge-charge repulsion (FIG. 2H). Finally, F423 and F369 in the JPU-B9-bound LC/A would sterically clash with T200 and M202 of SNAP25 and discourage substrate docking (FIG. 2H). These data suggest that JPU-B9 weakly inhibits SNAP25 activity by stabilizing LC/A in a conformation that is unfavorable for SNAP25 binding and cleavage.

### VHHs Target the Anchoring Site II

VHHs JPU-C1 and JPU-G3 both bind to the anchoring site II of LC/A and inhibit LC/A protease (Table 3; FIG. 10). JPU-C1 interacts extensively with LC/A involving all three CDRs, displaying a large BSA of 1010 Å² and an SC score of 0.686 (FIG. 15).

Interestingly, their CDR3s engage overlapping epitopes near the 160-loop of LC/A in nearly opposite orientations. As a result, the three LC/A-binding residues are in reverse orientation on JPU-C1 (residues¹⁰¹LED¹⁰³) and JPU-G3 (residues¹⁰⁴DEL¹⁰⁶) (FIGS. 3A-3F). JPU-G3 binds LC/A with BSA of 916 Å² and a lower SC score of 0.629, which is mainly mediated by its CDR3 (FIG. 15). The weaker binding of JPU-G3 in comparison to JPU-C1 may explain its lower protease inhibition potency.

Further structural analysis indicates that the CDR3 of JPU-C1 and JPU-G3 use a similar mechanism to interact with the SNAP25-binding residues on LC/A and therefore to preclude SNAP25 binding even though their CDR3 reaches into the anchoring site II from opposite orientations (FIGS. 3C and 3F). For example, L101 of JPU-C1 or L106 of JPU-G3 should prevent 1192 of SNAP25 from binding to F168 of LC/A. E102 of JPU-C1 or E105 of JPU-G3 form H-bonds with T176 of LC/A and will interfere with its binding to D193 of SNAP25. Both F168 and T176 are known to be crucial for the catalysis of SNAP25 cleavage [32]. Additionally, D103 of JPU-C1 forms salt bridges with R231 and R177 of LC/A while D104 of JPU-G3 forms a salt bridge with R231 of LC/A, both thereby interfering with LC/A binding to E194 of SNAP25. Taken together, the structural findings described here suggest that these two VHHs inhibit LC/A activity by blocking its binding to SNAP25 anchoring site II thereby inhibiting its subsequent cleavage.

### VHHs Target the Anchoring Site I

JPU-A11 and JPU-D12 bind overlapping epitopes at the anchoring site I of LC/A (FIGS. 4A-4F), and both are potent LC/A inhibitors (Table 3; FIG. 10). Binding of JPU-D12 buries an interface area of 768 Å² per molecule with a relatively high SC score of 0.775, while JPU-A11 interaction leads to a BSA of 984 Å² and an SC score of 0.671 (FIG. 15). JPU-D12-LC/A binding is strengthened by an extensive 17 H-bonds and 5 salt bridges. Structural analysis showed that the CDR1 and CDR3 of JPU-D12 form a convex surface that docks to a concave pocket of LC/A primarily composed of two helices (residues L310-Y321,D102-R113) and a few neighboring loops (FIGS. 4A and 4B). Specifically, F99 and L100 on the CDR3 loop of JPU-D12 occupy a pocket on LC/A containing V316, I115, R113, V112 and should thereby prevent the binding of I171 of SNAP25; E31 on the CDR1 loop of JPU-D12 forms a salt bridge and a hydrogen bond with H39 and N40 of LC/A, respectively, thus making it inaccessible to E170 of SNAP25 (FIG. 4C). JPU-A11 binds LC/A mainly through its CDR3 loop involving fewer electrostatic interactions (10 H-bonds and 1 salt bridge) in comparison to JPU-D12 (FIG. 15). When binding to LC/A, Y108 of JPU-A11 occupies a surface hydrophobic patch on LC/A, which should inhibit the binding of I171 of SNAP25 to this area (FIG. 4F). The higher SC score and more extensive electrostatic interactions of JPU-D12 likely explain its greater potency than JPU-A11 to inhibit LC/A activity (FIG. 10).

### VHHs Target the α-Exosite of LC/A

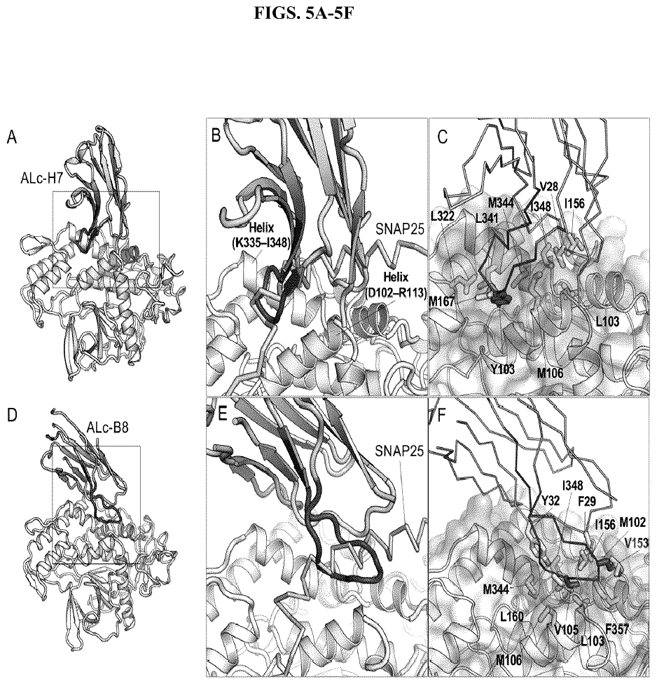

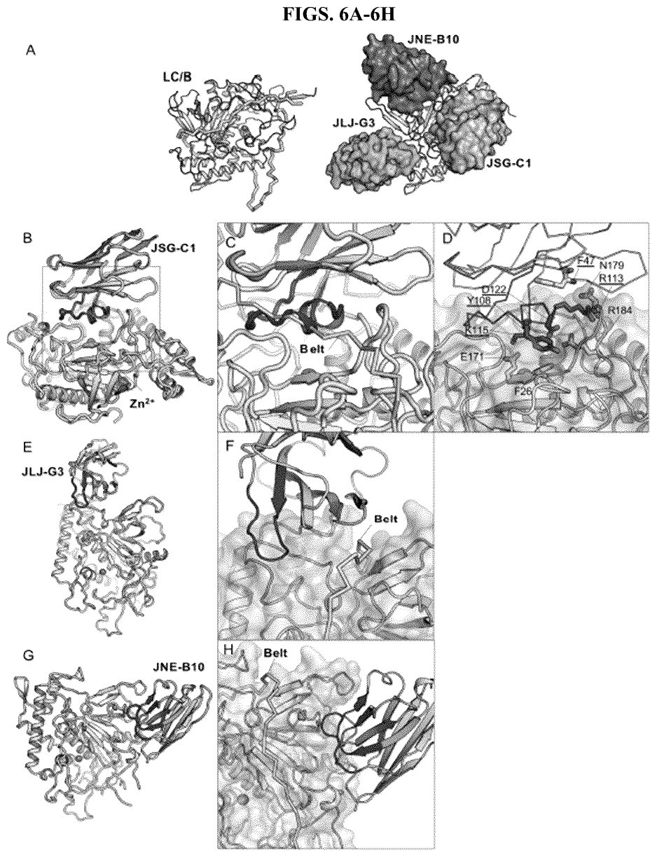

Structural studies revealed that three protease-inhibitory VHHs, ALc-H7, JPU-G11 and ALc-B8, bound to the α-exosite of LC/A. ALc-H7 and JPU-G11 have identical CDR3 sequences and thus not surprisingly recognize LC/A in an almost identical manner (FIG. 1A), so only ALc-H7 will be discussed further. ALc-H7 binds to LC/A and buries a molecular surface area of 715 Å² and displays a high SC score of 0.764 (FIG. 15). The CDR1 and CDR3 of ALc-H7 form a clamp that binds extensively to two a helices of LC/A (residues K335-I348, residues D102-R113) constituting the α-exosite (FIGS. 5A and 5B). V28 of the CDR1 of ALc-H7 interacts with residues L103 and I348 of LC/A which should thus prevent binding of I156 of SNAP25, while Y103 of CDR3 of ALc-H7 interacts with M106, M344, L341, L322 of LC/A and should prevent binding of M167 of SNAP25 (FIG. 5C). Therefore, ALc-H7 and JPU-G11 should strongly compete with SNAP25 for binding to the α-exosite of LC/A.

The ALc-B8-binding epitope on LC/A partially overlaps with that of ALc-H7, but ALc-B8 employs a different binding mode. The interaction of ALc-B8 with LC/A results in a BSA of 777 Å² per molecule and a SC score of 0.739 (FIG. 15). All CDRs of ALc-B8 contribute to LC/A recognition (FIGS. 5D and 5E). It was found that F29, Y32, M102 and V105 of ALc-B8 bound a hydrophobic patch on LC/A that is formed by M106, L103, F357, M344 and I348 and should compete for LC/A binding to SNAP25 residues V153, I156, and L160 (FIG. 5F). It was observed that a reported VHH Aal (PDB-ID: 3K3Q) has an epitope overlapping with ALc-B8 and ALc-H7 [33], but Aal has a smaller BSA. Together they reveal the diverse binding mechanisms that VHHs can employ for binding to the α-exosite epitope. Recent studies showed ALc-B8, when fused to an atoxic BoNT delivery vehicle, could protect mice, guinea pigs, and monkeys against BoNT/A1 intoxication even when delivered following the onset of symptoms of botulism [9, 10]. These results indicate that α-exosite binding VHHs can effectively inhibit cytosolic LC/A1 activity in vivo.

### Structures and Inhibition Mechanisms of Anti-LC/B VHHs

BoNT/B is the second most prevalent cause of human botulism behind BoNT/A, and its toxicity results from cytosolic LC/B cleavage of VAMP1 or VAMP2 at ⁷⁶QF⁷⁷ [34]. Although the structure of LC/B-VAMP remains unresolved, the structure of the LC/F-VAMP complex suggests that VAMP likely recognizes multiple anchoring points on LC/B along the shallow groove covered by the heavy chain belt in the holotoxin (FIG. 6A) [35]. Prior mutagenesis studies support the concept that, like LC/A, the substrate-binding pocket of LC/B likely aligns closely to how the heavy chain belt is associated in the holotoxin [34, 36, 37]. Truncation studies demonstrated that only 25 residues of VAMP2 (aa 61-85) were required for binding to LC/B while 57 residues (aa 141-202) of SNAP25 are required for LC/A binding [34, 36, 38]. Perhaps because of the relatively small LC/B-binding region in VAMP, competition studies with the full panel of LC/B-binding VHHs as described herein demonstrated that all protease inhibitory VHHs recognized only two non-overlapping epitopes. All of the most potent VHH inhibitors were members of the same competition group, in which JSG-C1 represents one of the most potent inhibitors of LC/B identified as described herein (Table 4; FIG. 11). For structural studies, JSG-C1 was selected, as well as the previously identified JLJ-G3 [8] that is a moderate LC/B protease inhibitor that binds to a distinct, non-overlapping epitope. A third VHH, JNE-B10, was also included in this study, as it binds at a third non-competing site and displays no protease inhibition activity, but unexpectedly provided protection from BoNT/B when delivered by an atoxic BoNT delivery vehicle to post-symptomatic animals [9](Table 4).

The binding of JSG-C1 to LC/B buries an interface area of 1063 Å² with a high SC score of 0.721 (FIG. 15). Structural analysis showed that JSG-C1 binding did not cause any structural changes on LC/B (PDB code 2ETF). The binding was mainly mediated by the long CDR3 that folds into a loop with a helical structure at the tip (FIG. 6B). JSG-C1 binds extensively through electrostatic interactions involving 21 H-bonds and 3 salt bridges to a pocket close to the active site of LC/B (FIG. 14). More specifically, D112 and K115 of JSG-C1 form salt bridges with R184 and E171 of LC/B, respectively; Y108 of JSG-C1 interacts hydrophobically with F26 of LC/B, and R113 and F47 are hydrogen bonded with N179 of LC/B (FIG. 6D). It was previously found that all of these LC/B residues involved in VHH binding are important for the catalysis of VAMP2 cleavage by LC/B [37]. The binding characterizations provided herein reveal that the binding of JSG-C1 to LC/B occludes VAMP from engaging at the cleavage pocket of LC/B (FIG. 6C).

JLJ-G3 is the only BoNT protease inhibitor VHH molecule identified to date that is able to bind both the isolated protease domain and the holotoxin. The crystal structure shows that JLJ-G3 binding buries an interface area of 647 Å² with an SC score of 0.695 on LC/B (FIG. 15; FIGS. 6E and 6F). Interestingly and as predicted by its binding to holotoxin, the JLJ-G3 binds outside the heavy chain belt-binding areas which is also believed to mimic the VAMP2-binding regions. However, the CDR3 and FR2 of JLJ-G3 form a concave surface that captures two surface loops of LC/B adjacent to the VAMP2-binding groove which should cause side-to-side clashes with the N-terminal region of VAMP2 when it binds to the substrate-binding pocket of LC/B. Therefore, JLJ-G3 is able to block VAMP2 binding to LC/B through side-to-side clashes without directly occupying the VAMP2-binding interface. In contrast to JLJ-G3, the JNE-B10-binding epitope on LC/B is not involved in substrate binding or in the interaction between LC/B and the heavy chain belt (FIGS. 6G and 611), consistent with its lack of protease inhibition activity as demonstrated in the assays described herein.

### Example 5: Identification of VHHs with Broad BoNT/A and BoNT/B Subtype Specificities

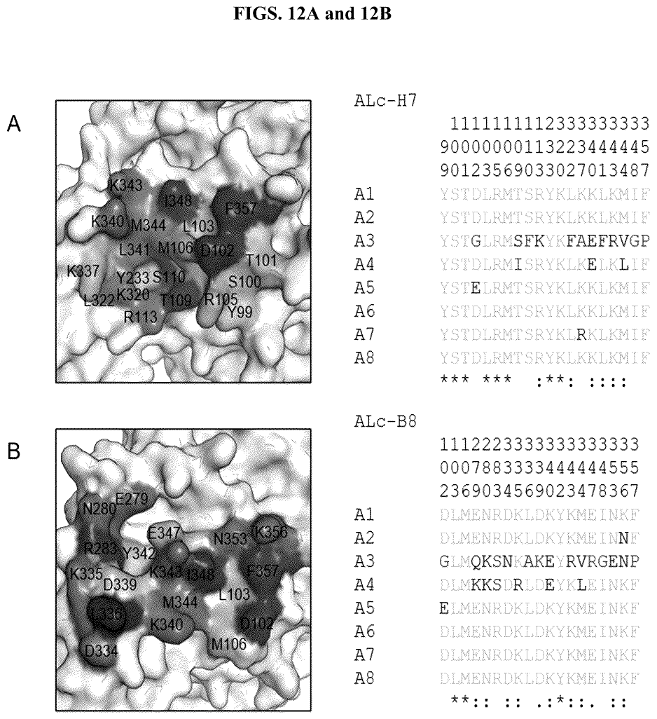

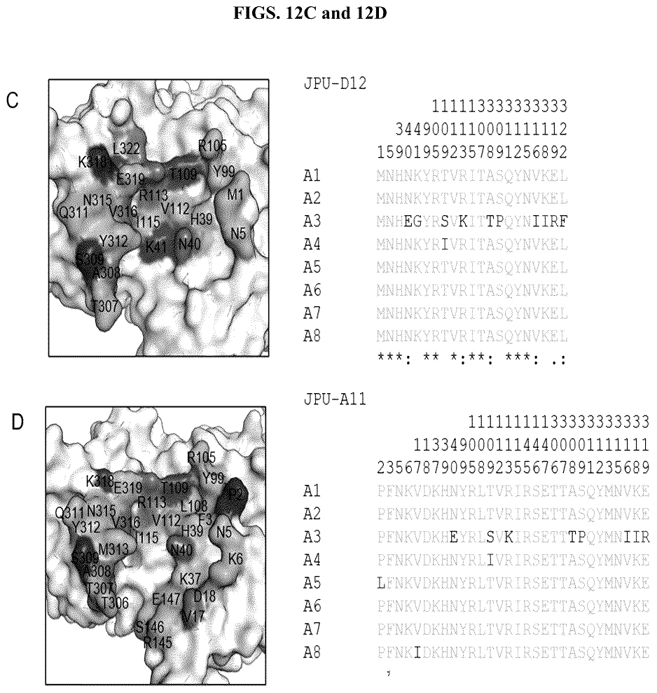

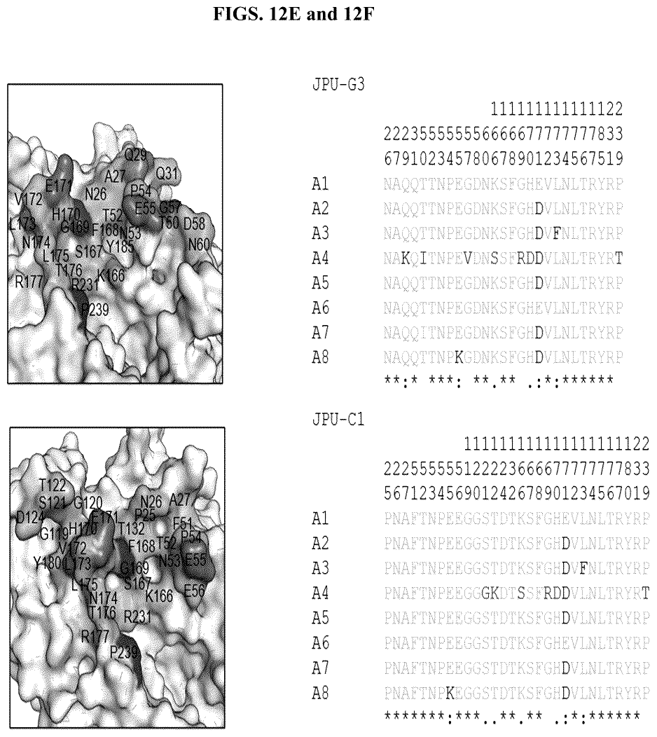

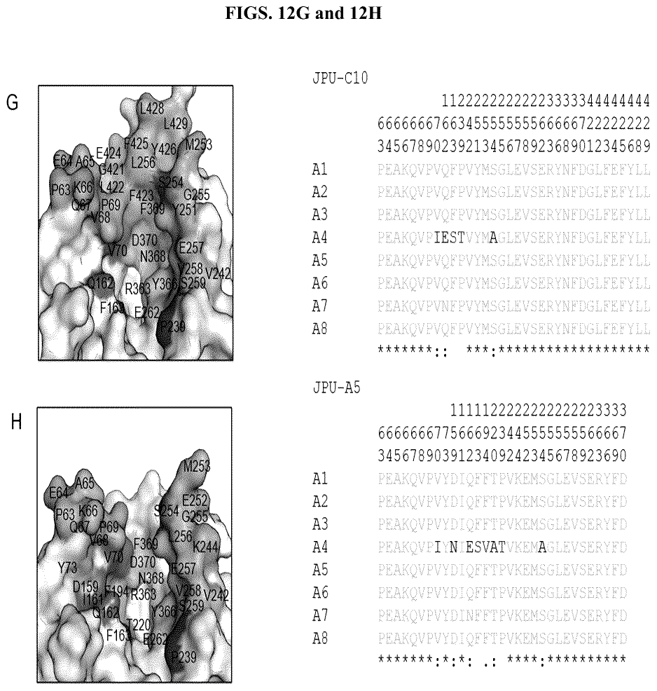

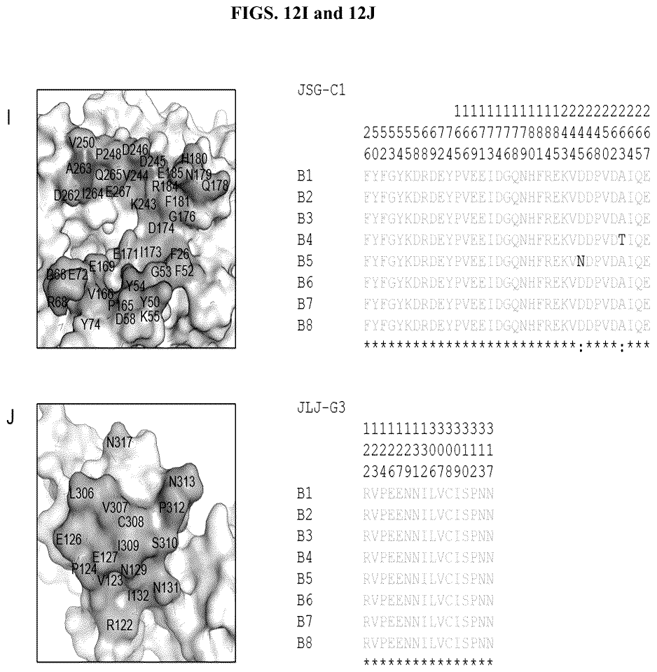

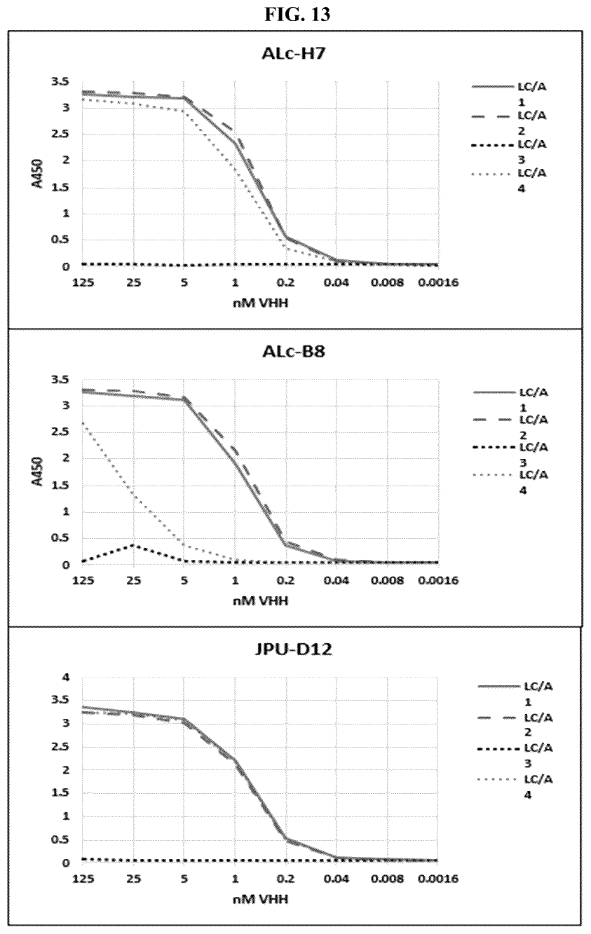

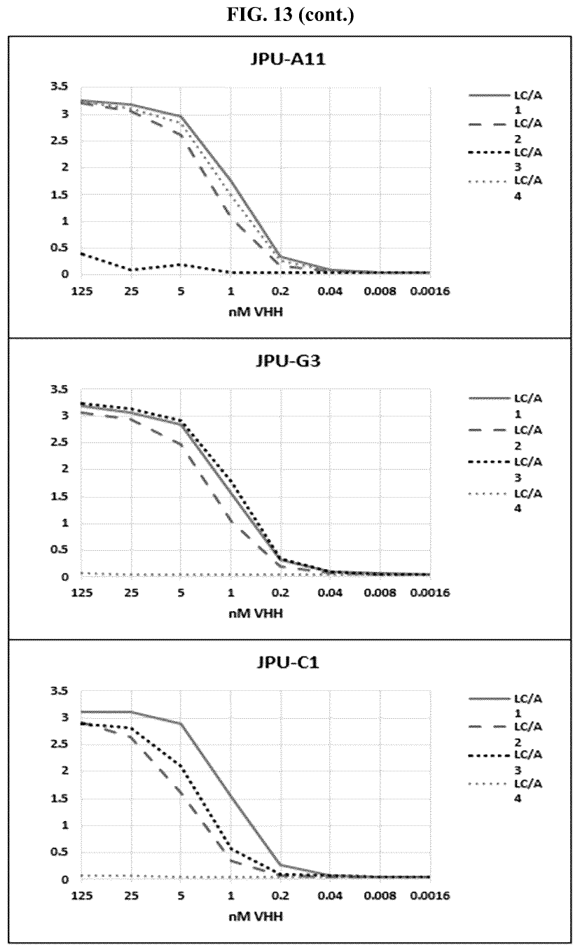

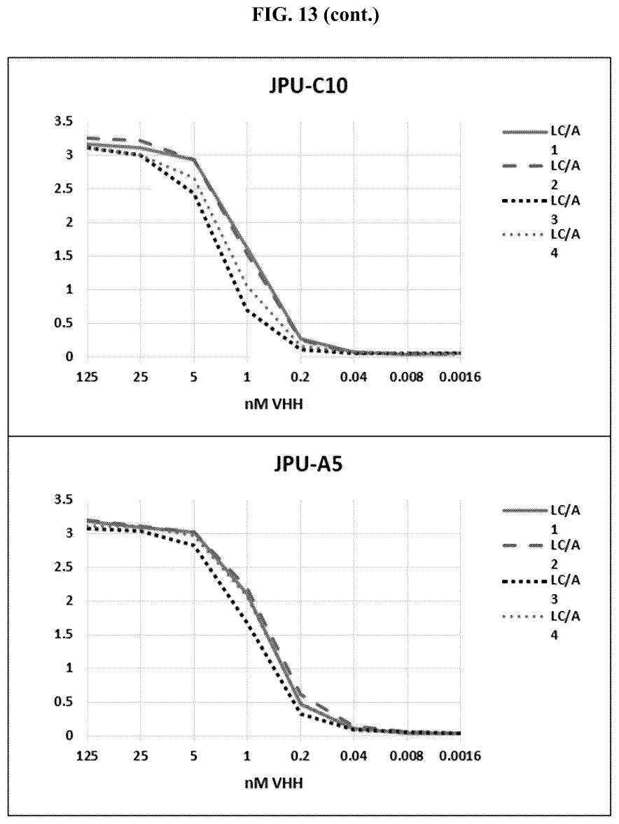

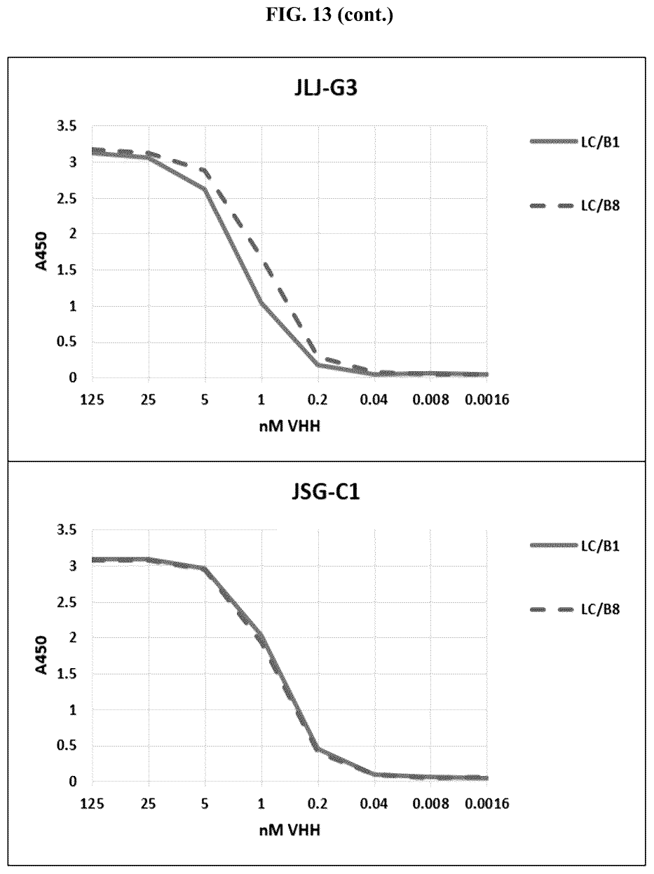

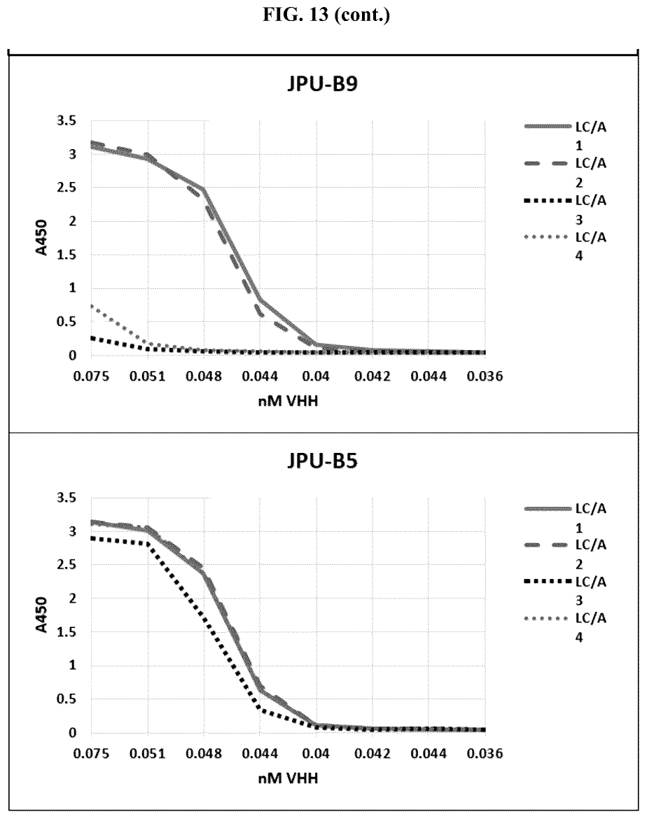

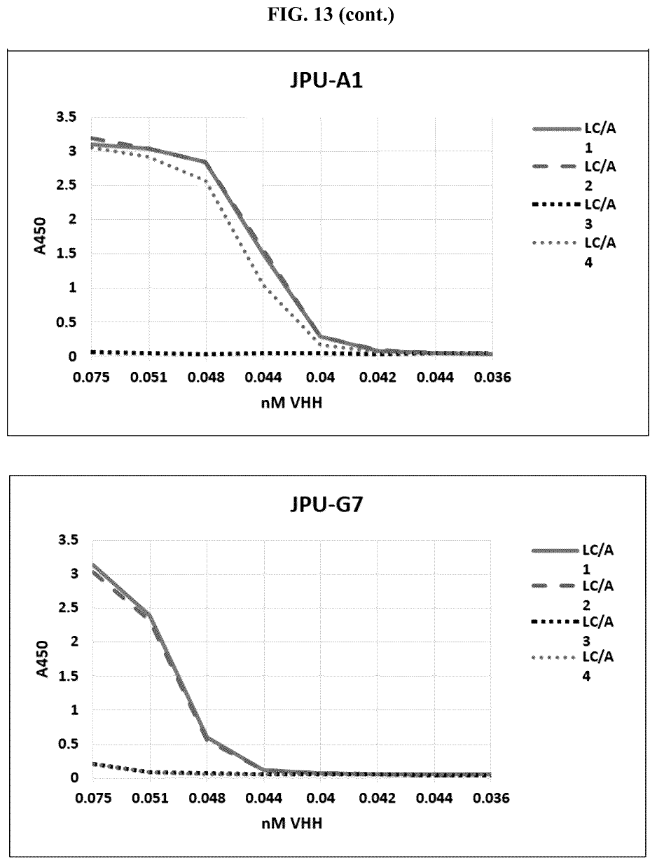

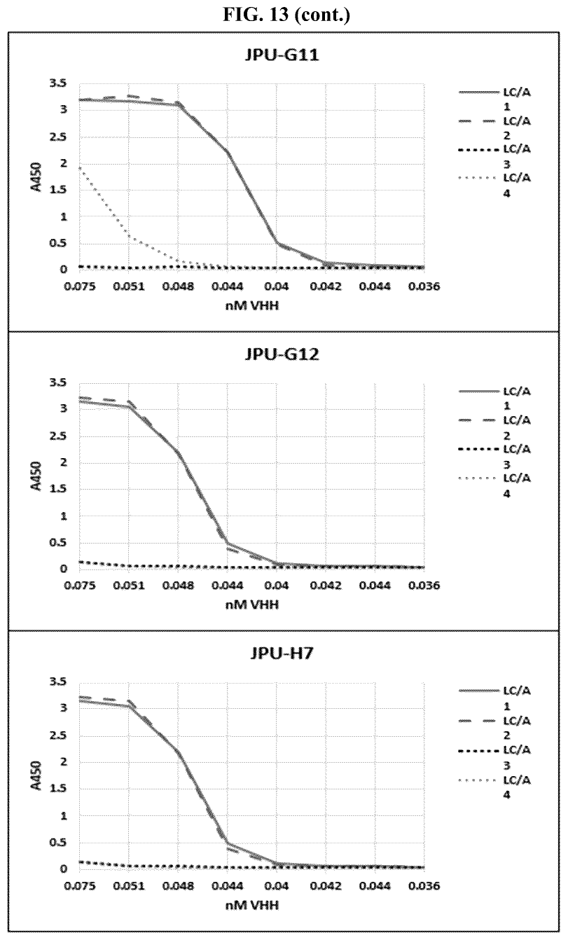

There are eight BoNT/A subtypes reported to date and the subtype A3 and A4 are the most divergent from A1, sharing primary sequence identity of 82.7% and 89.3%, respectively (FIG. 7A). The sequence conservation of epitopes among BoNT/A subtypes were analyzed for the potent inhibitor VHHs, and the impact on antibody binding was evaluated. The sequences near the α-exosite of A3 were found to be particularly divergent from those in A1. It is thus not surprising that the epitopes of ALc-B8 and ALc-H7 on LC/A1 have only 21% and 40% identity with the equivalent area on LC/A3 (FIGS. 7C and FIGS. 12A and 12B), indicating that these two VHHs would bind poorly to LC/A3. These predictions were validated by dilution ELISAs which demonstrated that ALc-B8 and ALc-H7 bound to LC/A3 with EC₅₀ values >125 nM (FIG. 13). In other examples, the sequences of the anchoring site I of LC/A3 have diverged significantly from that of LC/A1 (FIG. 7C), suggesting that JPU-A11 and JPU-D12 would bind poorly to LC/A3 (FIGS. 12C and 12D). Furthermore, the sequences of the anchoring site II of LC/A4 are divergent from subtype A1, and the ELISA data support the structural predictions that both JPU-C1 and JPU-G3 would bind poorly to LC/A4 (FIGS. 12E and 12F).

In contrast to the SNAP25-binding sites discussed above, the β-exosite region on LC/A is highly conserved across different A subtypes. Therefore, the epitopes of the two β-exosite-targeting VHHs, JPU-A5 and JPU-C10, are >80% conserved in all BoNT/A subtypes (FIGS. 12G and 12H). Consistent with these structural findings, ELISA studies support their broad specificity for LC/A subtypes (FIG. 13). These findings indicated that these two β-exosite-binding VHHs can be advantageous candidates for use as biomolecular antidotes as a result of their high potency to inhibit the LC/A protease and their broad specificity for all known BoNT/A subtypes. LC/B is far less variable than LC/A in nature, despite the eight natural subtypes identified to date, as these subtypes contain few amino acid differences as shown in FIG. 7B. The subtype that is most divergent from LC/B1 is LC/B8, which has 98.2% identity. Structural studies predict that the JSG-C1 binding site on LC/B is highly conserved (only 1 conservative amino acid change in B4 and B5), and the JLJ-G3 binding site is identical across all BoNT/B subtypes (FIGS. 7C and FIGS. 12I and 12J). Binding studies confirmed that both VHHs bound effectively to both B1 and B8 subtypes (FIG. 13). Taken together, these results with LC/A and LC/B VHHs demonstrate that structural studies of VHHs in complex with their targets can have significant value in predicting the range of VHH binding to natural target subtypes.

### Example 6

Botulinum neurotoxins are dangerous biothreat agents for which no post-intoxication antidote is available, and this results in a serious public health vulnerability. In a recent development, VHHs targeting LC/A or LC/B fused to an atoxic BoNT delivery vehicle were found capable of preventing death in mice, hamsters and primates that were treated post-symptomatically after receiving a lethal dose of BoNT/A or BoNT/B [9, 10]. The above Examples show the results of the identification and structural characterization of a large panel of VHHs as antidote candidates that bind to diverse epitopes on their LC/A or LC/B targets. In addition, studies with these VHHs revealed diverse mechanisms by which antibodies can inhibit BoNT protease activity. The findings provide the development and refinement of biomolecular antidotes for treating post-symptomatic botulism paralysis.

Because VHHs typically bind to conformational epitopes [13], it is critical to maintain the structure of the target when assaying VHH binding activity and function. This is particularly important in the case of BoNT protease domains as was reported recently for LC/E [7]. Based on VHH recognition, the conformation of LC/E protease appeared to change substantially when the protein was coated onto plastic, such that some VHHs recognized only coated LC/E, while other VHHs recognized only antibody-captured LC/E. This ‘plasticity’ of BoNT proteases may be related to their need to partially unfold during the translocation process from the endosome into the cytosol. To minimize conformational effects during the identification and characterization of the VHH polypeptides that bound to LC/A and LC/B as described herein, the panning and screening processes were performed using antibody-captured proteases. In both cases, the diversity of the VHH panels that were identified was substantially improved compared to earlier efforts that employed plastic-coated proteases [12]. Furthermore, most of the newly identified VHHs displayed higher binding affinities for captured proteases compared to plastic-coated proteases, and for some VHHs this difference was dramatic (Tables 3 and 4). It was concluded that LC/A and LC/B, similar to LC/E, undergo significant conformational deformity when coated to a hydrophobic plastic surface, thereby highlighting the value of maintaining conformational integrity of the target when performing VHH antibody selection and characterization as described herein.

The availability of large panels of new VHHs binding to LC/A and LC/B identified as described herein provide reagents that were able to probe a large portion of the surface of each protease for their functional role in SNARE protein recognition and cleavage (FIGS. 1 and 7). BoNT proteases are remarkably specific for their different SNARE protein substrates and this specificity is considered to be a result of the large region of interactions between these proteases and their substrates [15, 35]. For LC/A, protease inhibiting VHHs were identified that bound to all four of the reported SNAP25-binding sites, called the α- and β-exosites, and the anchoring sites I and II. Of note, JPU-A5 and JPU-C10 bind to the β-exosite of LC/A, which is a deep cleft at the active site of the protease, which is difficult to access by conventional antibodies [39, 40]. The findings described herein showed that LC/A protease function was inhibited when a VHH interfered with SNAP25 binding to any one of these four identified sites of interaction. Thus, the results described in the Examples herein strongly support the essential roles of all four of the SNAP25-binding sites for LC/A cleavage to occur. In contrast to LC/A, only two non-overlapping sites were identified on LC/B at which protease inhibiting VHHs could bind and interfere with VAMP binding. This finding is consistent with reports that VAMP binds to LC/B through a smaller interface that is yet to be fully characterized [34, 36].

Another major finding directly associated with the results described herein is new insight into the structure and function of the enigmatic carboxyl terminal region of LC/A. The full-length LC/A (fLC/A) including this region was previously considered to be un-crystallizable because it is flexible and prone to aggregation [24]. Herein, two VHHs (JPU-C10 and JPU-B9) were identified, both with epitopes that include the C-terminal region of fLC/A, which aided in determining the structures of fLC/A for the first time. It was found that the LC/A C-terminus dynamically altered between at least two different conformations, which likely plays an important role in facilitating SNAP25 binding and release [21, 41].

BoNTs are found in nature as at least seven different serotypes, and within each serotype are also found a substantial number of subtype variants. For therapeutic botulism antitoxins or antidotes to be broadly effective, they must be capable of protecting individuals from this diverse array of potential threats. VHH antibodies should optimally have broad specificity for natural subtype variability. Structural studies of VHHs bound to BoNT targets can be used to predict their subtype specificity, as is supported by the results demonstrated in the above Examples. By knowing the critical amino acids involved in the binding of many VHHs to their targets, their subtype specificity was predicted, and the predictions were successfully verified through binding studies. It was further demonstrated in the above Examples that by creating large panels of VHH polypeptides that bound to BoNT proteases, it was possible to identify a subset of VHH polypeptides (VHHs) that was both highly potent and broadly active as protease inhibitors. These VHHs (e.g., JPU-A5 and JPU-C10 for LC/A, JSG-C1 for LC/B) can advantageously serve as components for next generation biomolecular antidotes that should provide improved efficacy and broader variant specificity for post-exposure treatments for intoxication by the two most common threat agents, BoNT/A or BoNT/B.

### Example 7: Materials and Methods

### VHH Discovery and Identification

Two alpacas were immunized with purified LC/A protein and two alpacas were immunized with LC/B as previously reported [8, 12]. VHH-display phage libraries were prepared from immune PBLs obtained from each pair of immunized alpacas using standard lab procedures [44]. VHHs with affinity for native LC/A were selected by panning on LC/A immobilized by capture with the previously-isolated ALc-B8 VHH [12] coated onto plastic, and this resulted in selection of the JPU series of VHHs (Table 3). LC/B-binding VHHs were selected in two processes. The initial VHHs were obtained by capturing LC/B to plastic with BLc-B10 VHH [12], which led to the selection of several new LC/B-binding VHHs (JND series in Table 4). BLc-B10 proved to poorly capture LC/B; therefore, the discovery process was repeated using JND-E4 as the capture VHH, which led to selection of many additional LC/B-binding VHHs (JSG series in Table 4). Screening for LC-binding VHHs was performed by ELISAs employing VHH-captured LC/A or LC/B. All VHH panning and screening were performed using standard lab procedures [44].

All procedures using alpacas were conducted in accordance with the guidelines approved by the Institute Animal Care and Use Committee at Tufts Cummings School of Veterinary Medicine.

### Characterization of VHH Binding Properties

VHHs identified in the discovery process were subcloned into the pET-32 expression vector, expressed as fusions to E. coli thioredoxin and purified by standard lab procedures [44]. Binding properties were assessed by dilution ELISAs employing LC/A or LC/B proteases (0.5 or 1 μg/ml) that were coated onto Nunc MAXISORP™ or Costar tissue culture plastic, or captured to Nunc MAXISORP™ plastic using various protease-binding VHHs (coated at 2.5 or 5 μg/ml). In some cases, LC/A or LC/B were expressed as strep-tag fusion proteins and employed in ELISAs in which the tagged proteases were captured onto streptactin plates (IBA Lifesciences). Dilution ELISAs were performed as previously described [8] and apparent binding affinities under each of the ELISA conditions were assessed by estimating the EC₅₀ values, i.e., the VHH polypeptide concentration that resulted in 50% peak binding.

### Protease Inhibition Assays

All VHHs were tested for their potencies to inhibit LC/A and LC/B protease activity. The assays employed multiple substrates. For SNAP25 cleavage, either recombinant YFP/SNAP25/CFP (BoTest A/E reporter, BioSentinel) or Repcon5 [7] was employed. For VAMP cleavage, a recombinant YFP/VAMP/CFP protein produced from an expression plasmid (kindly provided by Dr. George Oyler or Repcon5) was employed. Substrate cleavage under various conditions and times was assessed by performing western blots using anti-GFP (Santa Cruz) or anti-hexahistidine (“hexahistidine” disclosed as SEQ ID NO: 129) (Santa Cruz) for detection of the reporter and cleavage products as previously described [7]. All assays of protease-inhibitory VHHs were repeated at least three times with different VHH:protease ratios and different times of incubation to generate the potency assessments presented in Table 3 and Table 4.

### Cloning, Expression, and Purification of Recombinant Proteins for Crystallization

LC/B(M1-S425), LC/B(M1-K441), anti-BoNT/A VHHs (JPU-G11, JPU-D12, JPU-A11, JPU-C1, JPU-G3, JPU-A5, JPU-B9, and JPU-C10), and anti-BoNT/B VHHs (JNE-B10, JLJ-G3, and JSG-C1) were cloned into pGEX-6p-1 for expression following the N-terminal GST and a PreScission cleavage site. LC/A(M1-T420), LC/A (P2-K438), ALc-H7, ALc-B8, ciA-D12, and ciA-F12 were expressed and purified as described previously [8, 12, 45].

GST-tagged LC/A, LC/B, and VHHs (JPU-G11, JPU-D12, JPU-A11, JPU-C1, JPU-G3, JPU-B9, JPU-C10, JNE-B10, JLJ-G3, and JSG-C1) were expressed in E. coli strain 13L21-Star (DE3) (Invitrogen). GST-tagged JPU-A5 was expressed in the E. coli strain Origami B (DE3) (Novagen). Bacteria were cultured at 37° C. in LB medium containing ampicillin. The temperature was reduced to 18° C. when OD₆₀₀ reached ˜0.6. Expression was induced with 1 mM IPTG and continued at 18° C. for 16 hours. Cells were harvested by centrifugation and stored at −20° C. until use.

For protein purification, bacteria were re-suspended in a buffer containing 50 mM Tris (pH 8.0), 400 mM NaCl, and 0.4 mM PMSF and lysed by sonication. All GST-tagged proteins were purified using Glutathione Sepharose 4B resins (GE Healthcare) in 50 mM Tris (pH 8.0), 400 nM NaCl, and eluted from the resins after on-column cleavage using PreScission protease. The proteins were further purified by Superdex-200 Increase or Superdex-75 SEC in 10 mM HEPES (pH 7.4) and 150 mM NaCl. The LC-VHH complexes were made by mixing the purified LC and VHHs at a molar ratio of 1:1.5 for 1 hour on ice, followed by purification using Superdex-200 Increase SEC (10 mM HEPES, pH7.4, 150 mM NaCl, and 5 μM ZnSO₄). Each protein complex was concentrated to ˜5 mg/ml using Armicon Ultra centrifugal filters (Millipore) and stored at −80° C. until further characterization or crystallization.

### Crystallization

Initial crystallization screens were performed using a Gryphon crystallization robot (Art Robbins Instruments) and high-throughput crystallization screen kits (Hampton Research and Qiagen). Extensive manual optimizations were performed at 18° C. when proteins were mixed with reservoir solution at 1:1 ratio.
1. The best single crystals of sLC/A-JPU-A5-ALc-H7-JPU-C1-JPU-D12-ciA-F12 were grown by the sitting-drop vapor diffusion method at a protein concentration of 8 mg/ml with a reservoir solution containing 20% 2-propanol, 0.1 M sodium citrate, 20% PEG4000, pH 5.6.
2. Crystals of fLC/A-ALc-B8-JPU-C10-JPU-G3-JPU-D12-ciA-F12-ciA-D12 were obtained by the sitting-drop vapor diffusion method at a protein concentration of 6 mg/ml with a reservoir solution containing 2.4 M sodium malonate, 0.1 M Bis-Tris, pH 7.0.
3. Crystals of fLC/A-JPU-B9-JPU-A11-JPU-G11 were grown by the sitting-drop vapor diffusion method at a protein concentration of 5 mg/ml with a reservoir solution containing 25% PEG 4000, 0.1 M sodium cacodylate, pH 6.5.
4. Crystals of sLC/B-JLJ-G3-JNE-B10 were obtained by the sitting-drop vapor diffusion method at a protein concentration of 5 mg/ml with a reservoir solution containing 0.2 M Kl, 25% PEG 4000, 0.1 M MES, pH 6.5.
5. Crystals of fLC/B-JSG-C1 were obtained by the sitting-drop vapor diffusion method at a protein concentration of 5 mg/ml with a reservoir solution containing 20% PEG 4000, 0.15 M ammonium sulfate, 0.1 M Hepes, pH 7.0.

### Data Collection and Structure Determination

All crystals were cryoprotected in their original mother liquor supplemented with 20-25% (v/v) ethylene glycol. The X-ray diffraction data for the crystals were collected at 100 K at the NE-CAT beamline 24-ID-E, Advanced Photon Source (APS). The data were processed with iMOSFLM [46] or XDS as implemented in RAPD (github-dot-com/RAPD/RAPD)[47]. Data collection statistics are summarized in FIG. 15. Structures of the sLC/A-JPU-A5-ALc-H7-JPU-C1-JPU-D12-ciA-F12, fLC/A-ALc-B8-JPU-C10-JPU-G3-JPU-D12-ciA-F12-ciA-D12, and fLC/A-JPU-B9-JPU-A11-JPU-G11 complexes were determined by molecular replacement using the Phaser software [48] with LC/A (PDB code 1XTF) [15] and the homology models of VHHs that were built based on a VHH in PDB 5L21 [49] as the search models. Structures of the complexes were solved by molecular replacement with Phaser using LC/B (PDB code 2ETF) and the homology models of the VHHs as the search models. Manual model building and refinement were performed in COOT [50], PHENIX [51], and CCP4 suite [52] in an iterative manner. The refinement progress was monitored with the free R value using a 5% randomly selected test set [53]. The structures were validated through MolProbity [54] and showed excellent stereochemistry. Structural refinement statistics are listed in FIG. 15. All structure figures were prepared with PyMol (worldwideweb-pymol-org).

### Data Code Availability

Atomic coordinates and structure factors for sLC/A-JPU-A5-ALc-H7-JPU-C1-JPU-D12-ciA-F12, fLC/A-ALc-B8-JPU-C10-JPU-G3-JPU-D12-ciA-F12-ciA-D12, fLC/A-JPU-B9-JPU-A11-JPU-G11, sLC/B-JLJ-G3-JNE-B10, fLC/B-JSG-C1 have been deposited in the Protein Data Bank under accession codes 7L6V, 7M1H, 7LZP, 5L2C, and 7NA9, respectively. (See, e.g., Lam, K-h. et al., 2022, PLoS Path, 18(1); pages 1-12; e1010169).

### Additional Embodiments

Embodiment 1. A polypeptide that specifically binds to a light chain (LC) protease domain of Botulinum neurotoxin (BoNT) serotype A (LC/A toxin or LC/A), or a LC/A-binding portion thereof, wherein the polypeptide comprises three complementarity determining regions (CDRs), CDR1, CDR2 and CDR3, and four VHH framework regions (FRs), FR1, FR2, FR3 and FR4, with the general structure FR1-CDR1-FR2-CDR2-FR3-CDR3-FR4,
   wherein the CDR1, CDR2, and CDR3 of the polypeptide comprise an amino acid sequence of the CDR1, CDR2, and CDR3, respectively, or a below-listed polypeptide, and the FR1, FR2, FR3 and FR4 comprise an amino acid sequence having at least 85% sequence identity to a polypeptide named as listed below and as described in the disclosure, Tables, Figures and Supplementary disclosure, Tables and Figures herein:

JPU-A1 (SEQ ID NO: 7)
JPU-A5 (SEQ ID NO: 8)
JPU-A11 (SEQ ID NO: 9)
JPU-B5 (SEQ ID NO: 10)
JPU-B9 (SEQ ID NO: 11)
JPU-C1 (SEQ ID NO: 12)
JPU-C10 (SEQ ID NO: 13)
JPU-D12 (SEQ ID NO: 14)
JPU-G3 (SEQ ID NO: 15)
JPU-G7 (SEQ ID NO: 16)
JPU-G11 (SEQ ID NO: 17)
JPU-G12 (SEQ ID NO: 18)
JPU-H7 (SEQ ID NO: 19)

Embodiment 2. A polypeptide that specifically binds to a light chain (LC) protease domain of Botulinum neurotoxin (BoNT) serotype B (LC/B toxin or LC/B), or a LC/B-binding portion thereof, wherein the polypeptide comprises three complementarity determining regions (CDRs), CDR1, CDR2 and CDR3, and four VHH framework regions (FRs), FR1, FR2, FR3 and FR4, with the general structure FR1-CDR1-FR2-CDR2-FR3-CDR3-FR4,
   wherein the CDR1, CDR2, and CDR3 of the polypeptide comprise an amino acid sequence of the CDR1, CDR2, and CDR3, respectively, or a below-listed polypeptide, and the FR1, FR2, FR3 and FR4 comprise an amino acid sequence having at least 85% sequence identity to a polypeptide named as listed below and as described in the disclosure, Tables, Figures and Supplementary disclosure, Tables and Figures herein:

JND-A12 (SEQ ID NO: 24)
JND-B4 (SEQ ID NO: 25)
JND-C7 (SEQ ID NO: 26)
JND-E4 (SEQ ID NO: 27)
JND-E5 (SEQ ID NO: 28)
JND-E9 (SEQ ID NO: 29)
JND-F3 (SEQ ID NO: 30)
JSG-B8 (SEQ ID NO: 31)
JSG-B10 (SEQ ID NO: 32)
JSG-C1 (SEQ ID NO: 33)
JSG-F6 (SEQ ID NO: 34)
JSG-G1 (SEQ ID NO: 35)
JSG-G10 (SEQ ID NO: 36)
JSG-G11 (SEQ ID NO: 37)

Embodiment 3. The polypeptide of Embodiment 1, wherein the polypeptide inhibits and/or neutralizes BoNT/A LC/A protease function or activity.
Embodiment 4. The polypeptide of Embodiment 2, wherein the polypeptide inhibits and/or neutralizes BoNT/B LC/B protease function or activity.
Embodiment 5 The polypeptide of any one of Embodiments 1 or 2, which is a camelid-derived single domain anti-LC/A or LC/B VHH antibody.
Embodiment 6. The polypeptide of any one of Embodiments 1-5, in the form of a dimer or multimer.
Embodiment 7. The polypeptide of any one of Embodiments 1-6, comprising one or more epitope tag sequences specifically bindable by an anti-epitope tag antibody or binding portion thereof.
Embodiment 8. A polypeptide that specifically binds to light chain (LC) protease domain of Botulinum neurotoxin (BoNT) serotype A (LC/A toxin or LC/A), wherein the polypeptide or an LC/A-binding portion thereof has at least 85%, 90%, 95%, or 98% amino acid sequence identity to an amino acid sequence of at least one of the polypeptides named as listed below and as described in the disclosure, Tables, Figures and Supplementary disclosure, Tables and Figures herein:

JPU-A1 (SEQ ID NO: 7)
JPU-A5 (SEQ ID NO: 8)
JPU-A11 (SEQ ID NO: 9)
JPU-B5 (SEQ ID NO: 10)
JPU-B9 (SEQ ID NO: 11)
JPU-C1 (SEQ ID NO: 12)
JPU-C10 (SEQ ID NO: 13)
JPU-D12 (SEQ ID NO: 14)
JPU-G3 (SEQ ID NO: 15)
JPU-G7 (SEQ ID NO: 16)
JPU-G11 (SEQ ID NO: 17)
JPU-G12 (SEQ ID NO: 18)
JPU-H7 (SEQ ID NO: 19)

Embodiment 9. A polypeptide that specifically binds to light chain (LC) protease domain of Botulinum neurotoxin (BoNT) serotype B (LC/B toxin or LC/B), wherein the polypeptide or an LC/B-binding portion thereof has at least 85%, 90%, 95%, or 98% amino acid sequence identity to an amino acid sequence of at least one of the polypeptides named as listed below and as described in the disclosure, Tables, Figures and Supplementary disclosure, Tables and Figures herein:

JND-A12 (SEQ ID NO: 24)
JND-B4 (SEQ ID NO: 25)
JND-C7 (SEQ ID NO: 26)
JND-E4 (SEQ ID NO: 27)
JND-E5 (SEQ ID NO: 28)
JND-E9 (SEQ ID NO: 29)
JND-F3 (SEQ ID NO: 30)
JSG-B8 (SEQ ID NO: 31)
JSG-B10 (SEQ ID NO: 32)
JSG-C1 (SEQ ID NO: 33)
JSG-F6 (SEQ ID NO: 34)
JSG-G1 (SEQ ID NO: 35)
JSG-G10 (SEQ ID NO: 36)
JSG-G11 (SEQ ID NO: 37)

Embodiment 10. The polypeptide of Embodiment 8 or 9, wherein conservative amino acid substitutions in the polypeptide comprise the at least 85%, at least 90%, at least 95%, or at least 98% amino acid sequence identity.
Embodiment 11. A polypeptide that specifically binds to the light chain (LC) protease domain of Botulinum neurotoxin (BoNT) serotype A (LC/A toxin or LC/A) or to the light chain (LC) protease domain of Botulinum neurotoxin (BoNT) serotype B (LC/B toxin or LC/B), wherein the polypeptide or an LC/A- or LC/B-binding portion thereof comprises or consists of a sequence selected from the group consisting of the LC/A- or the LC/B-binding VHH molecule sequences and as described in the disclosure, Tables, Figures and Supplementary disclosure, Tables and Figures herein.
Embodiment 12. The polypeptide of any one of Embodiments 8-11, wherein the polypeptide inhibits and/or neutralizes LC/A or LC/B activity.
Embodiment 13. The polypeptide any one of Embodiments 8-12, which is a camelid-derived single domain VHH antibody (VHH).
Embodiment 14. The polypeptide of any one of Embodiments 8-13, in the form of a dimer or multimer.
Embodiment 15. The polypeptide of any one of Embodiments 8-14, comprising one or more epitope tag sequences specifically bindable by an anti-epitope tag antibody or binding portion thereof.
Embodiment 16. The polypeptide of Embodiment 7 or claim 15, wherein the one or more epitope tag sequences comprises at least one of DELGPRLMGK (SEQ ID NO: 119) or GAPVPYPDPLEPR (SEQ ID NO: 120).
Embodiment 17. The polypeptide of any one of Embodiments 13-16, comprising two or more anti-LC/A or anti-LC/B VHH polypeptides, or LC/A- or LC/B-binding regions thereof, joined with one or more linker peptides.
Embodiment 18. The polypeptide of Embodiment 17, wherein the one or more linker peptides is selected from GGGGS (SEQ ID NO: 121); GGGGSGGGGSGGGGS (SEQ ID NO: 122), or a functional portion thereof; EPKTPKPQGGGGSGGGGSGGGGSQGVQSQVQLVE (SEQ ID NO: 123); EPKTPKPQ (SEQ ID NO: 124); or a combination thereof.
Embodiment 19. The polypeptide of any one of Embodiments 14-18, wherein the polypeptide is dimeric and comprises two anti-LC/A VHH polypeptides.
Embodiment 20. The polypeptide of Embodiment 19, wherein the two anti-LC/A VHH polypeptides are the same or different.
Embodiment 21. The polypeptide of any one of claims 14-18, wherein the polypeptide is dimeric and comprises two anti-LC/B VHH polypeptides.
Embodiment 22. The polypeptide of Embodiment 21, wherein the two anti-LC/B VHH polypeptides are the same or different.
Embodiment 23. The polypeptide of any one of Embodiments 14-18, wherein the polypeptide is dimeric and comprises an anti-LC/A VHH polypeptide and an anti-LC/B VHH polypeptide.
Embodiment 24. The polypeptide of any one of Embodiments 14-18, wherein the polypeptide is multimeric and comprises at least three anti-LC/A and/or anti-LC/B VHH polypeptides, same or different.
Embodiment 25. The polypeptide of Embodiment 24, wherein the multimeric polypeptide comprises at least three of the same or different anti-LC/A VHH polypeptides.
Embodiment 26. The polypeptide of claim 24, wherein the multimeric polypeptide comprises at least three of the same or different anti-LC/B VHH polypeptides.
Embodiment 27. The polypeptide of claim 24, wherein the multimeric polypeptide comprises at least three of a combination of the same or different anti-LC/A and anti-LC/B VHH polypeptides.
Embodiment 28. An isolated polynucleotide encoding the polypeptide of any one of Embodiments 1-13.
Embodiment 29. An isolated polynucleotide encoding the dimeric or multimeric polypeptide of any one of Embodiments 14-27.
Embodiment 30. A vector comprising the isolated polynucleotide of Embodiment 28 or 29.
Embodiment 31. The vector of Embodiment 30, wherein the vector is an expression vector.
Embodiment 32. The vector of Embodiment 31, wherein the expression vector is a viral or non-viral expression vector.
Embodiment 33. A host cell comprising the vector of any one of Embodiments 30-32.
Embodiment 34. A pharmaceutical composition comprising an effective amount of the polypeptide of any one of Embodiments 1-13, or an LC/A- or LC/B-binding fragment thereof, and a pharmaceutically acceptable excipient, carrier, or diluent.
Embodiment 35. A pharmaceutical composition comprising an effective amount of the polypeptide of any one of Embodiments 14-27, or a toxin-binding fragment thereof, and a pharmaceutically acceptable excipient, carrier, or diluent.
Embodiment 36. A pharmaceutical composition comprising an effective amount of the isolated polynucleotide of Embodiment 28 or 29, and a pharmaceutically acceptable excipient, carrier, or diluent.
Embodiment 37. A method of reducing, ameliorating, alleviating, abating, or eradicating intoxication of a cell by Botulinum neurotoxin serotype A (BoNT/A) and/or Botulinum neurotoxin serotype B (BoNT/B), the method comprising contacting the cell with an effective amount of the polypeptide of any one of Embodiments 1-27, or a pharmaceutical composition thereof, thereby reducing, ameliorating, alleviating, abating, or eradicating intoxication of the cell by the BoNT/A and/or BoNT/B.
Embodiment 38. A method of reducing, ameliorating, alleviating, abating, or eradicating intoxication of a cell by Botulinum neurotoxin serotype A (BoNT/A) and/or Botulinum neurotoxin serotype B (BoNT/B), the method comprising contacting the cell with an effective amount of the isolated polynucleotide of Embodiment 28 or 29, or a pharmaceutical composition thereof, or the vector of any one of claims 29-31, thereby reducing, ameliorating, alleviating, abating, or eradicating intoxication of the cell by the BoNT/A and/or BoNT/B.
Embodiment 39. The method of Embodiment 37 or 38, wherein the cell is in vitro.
Embodiment 40. The method of Embodiment 37 or 38, wherein the cell is in vivo.
Embodiment 41. A method of treating botulism or intoxication by LC/A toxin- and/or LC/B toxin-producing Botulinum microorganisms, and/or the symptoms thereof, the method comprising administering to a subject in need thereof an effective amount of the polypeptide of any one of Embodiments 1-27 or a pharmaceutical composition thereof, the isolated polynucleotide of Embodiment 28 or 29 or a pharmaceutical composition thereof, or the vector of any one of Embodiments 30-32 or a pharmaceutical composition thereof, thereby treating botulism or intoxication, and/or the symptoms thereof in the subject.
Embodiment 42. A method of reducing the severity of botulism or intoxication associated with infection by Botulinum microorganisms producing LC/A and/or LC/B toxins, and/or the symptoms thereof, in a subject who has, who is susceptible to, or who is at risk of having botulism or intoxication by Botulinum microorganisms, the method comprising administering to a subject in need thereof an effective amount of the polypeptide of any one of Embodiments 1-27 or a pharmaceutical composition thereof, the isolated polynucleotide of Embodiment 28 or 29 or a pharmaceutical composition thereof, or the vector of any one of Embodiments 30-32 or a pharmaceutical composition thereof, thereby reducing the severity of botulism or intoxication by the Botulinum microorganisms and/or the symptoms thereof in the subject.
Embodiment 43. A method of treating or preventing botulism or intoxication associated with infection by Botulinum microorganisms and/or the symptoms thereof in a subject, the method comprising administering to a subject in need thereof an effective amount of the polypeptide of any one of Embodiments 1-27 or a pharmaceutical composition thereof, the isolated polynucleotide of Embodiment 28 or 29 or a pharmaceutical composition thereof, or the vector of any one of Embodiments 30-32 or a pharmaceutical composition thereof, thereby treating or preventing botulism or intoxication by the Botulinum microorganisms and/or the symptoms thereof in the subject.
Embodiment 44. The method of any one of Embodiments 41-43, further comprising administering to the subject an anti-epitope tag antibody that specifically binds to an epitope tag, if present, and facilitates clearance of a complex of LC/A and/or LC/B bound to the anti-LC/A and/or anti-LC/B VHH polypeptide from the subject.
Embodiment 45. A method of treating botulism or intoxication by Botulinum microorganisms producing LC/A and/or LC/B neurotoxins, and/or the symptoms thereof in a subject, the method comprising administering to a subject in need thereof an effective amount of the pharmaceutical composition of any one of Embodiments 34-36, thereby treating botulism or infection or intoxication by Botulinum microorganisms of serotype A and/or B and/or the symptoms thereof in the subject.
Embodiment 46. The method of any one of Embodiments 37-45, wherein BoNT/A LC/A and/or BoNT/B LC/B protease function or activity is inhibited and/or neutralized.
Embodiment 47. A kit comprising the polypeptide of any one of Embodiments 1-27, or a pharmaceutical composition thereof, for treating or protecting against disease or intoxication and/or the symptoms thereof caused by Botulinum serotype A LC/A protease and/or Botulinum serotype B LC/B protease.
Embodiment 48. A kit comprising the isolated polynucleotide of Embodiment 28 or 29, or a pharmaceutical composition thereof, for treating or protecting against disease or intoxication and/or the symptoms thereof caused by Botulinum serotype A LC/A protease and/or Botulinum serotype B LC/B protease.
Embodiment 49. The kit of Embodiment 47 or 48, further comprising instructions for use.

Some of the data used in this work was provided and made available by the Regents of the University of California.

### REFERENCES

1. Nayak S U et al., Antimicrob Agents Chemother. 2014; 58(9):5047-53. Epub 2014/06/11.doi: 10.1128/AAC.02830-14. PubMed PMID: 24913160; PubMed Central PMCID: PMCPMC4135817.
2. Snow D M, et al., Antimicrob Agents Chemother. 2019. Epub 2019/10/09. doi: 10.1128/AAC.01270-19. PubMed PMID: 31591130.
3. Tomic M T, et al., Toxins (Basel). 2019; 11(4). Epub 2019/04/10. doi: 10.3390/toxins11040208. PubMed PMID: 30959899.
4. Garcia-Rodriguez C, et al., Toxins (Basel). 2018; 10(3). Epub 2018/03/02. doi: 10.3390/toxins10030105. PubMed PMID: 29494481; PubMed Central PMCID: PMCPMC5869393.
5. Fan Y, et al., PLoS One. 2017; 12(3):e0174187. Epub 2017/03/23. doi: 10.1371/journal.pone.0174187. PubMed PMID: 28323873; PubMed Central PMCID: PMCPMC5360321.
6. Mukherjee J, et al., PLoS ONE. 2012; 7(1):e29941. Epub 2012/01/13. doi: 10.1371/journal.pone.0029941.PubMed PMID: 22238680.
7. Tremblay J M, et al., Toxins (Basel). 2020; 12(10). Epub 2020/09/30. doi: 10.3390/toxins12100611. PubMed PMID: 32987745.
8. Lam K H, et al., Cell Rep. 2020; 30(8):2526-39 e6. Epub 2020/02/27. doi: 10.1016/j.celrep.2020.01.107. PubMed PMID: 32101733.
9. Miyashita S I, et al., Sci Transl Med. 2021; 13(575). doi: 10.1126/scitranslmed.aaz4197. PubMed PMID: 33408184.
10. McNutt P M, et al., Sci Transl Med. 2021; 13(575). doi: 10.1126/scitranslmed.abd7789. PubMed PMID: 33408188.
11. Dong J, et al., J Mol Biol. 2010; 397(4):1106-18. Epub 2010/02/09. doi: 10.1016/j.jmb.2010.01.070. PubMed PMID: 20138889; PubMed Central PMCID: PMCPMC2903050.
12. Tremblay J M, et al., Toxicon. 2010; 56(6):990-8. doi: 10.1016/j.toxicon.2010.07.003.
13. Pardon E, et al., Nat Protoc. 2014; 9(3):674-93. Epub 2014/03/01. doi: 10.1038/nprot.2014.039. PubMed PMID: 24577359; PubMed Central PMCID: PMCPMC4297639.
14. Brunger A T, et al., PLoS Pathog.2007; 3(9):1191-4. doi: 10.1371/journal.ppat.0030113. PubMed PMID: 17907800; PubMed Central PMCID: PMCPMC1994969.
15. Breidenbach M A, et al., Nature. 2004; 432(7019):925-9. PubMed PMID: 15592454.
16. Desmyter A, et al., Curr Opin Struct Biol. 2015; 32:1-8. Epub 2015/01/24. doi: 10.1016/j.sbi.2015.01.001. PubMed PMID: 25614146.
17. Silvaggi N R, et al., Chemistry & biology. 2007; 14(5):533-42. PubMed PMID: 17524984.
18. Fernandez-Salas E, et al., Proc Natl Acad Sci USA. 2004; 101(9):3208-13. Epub 2004/02/26. doi: 10.1073/pnas.0400229101 0400229101 [pii]. PubMed PMID: 14982988; PubMed Central PMCID: PMC365768.
19. Vagin O, et al, J Cell Sci. 2014; 127(Pt 15):3294-308. Epub 2014/06/13. doi: 10.1242/jes.146324. PubMed PMID: 24928902; PubMed Central PMCID: PMCPMC6518159.
20. Wang J, et al., J Biol Chem. 2011; 286(8):6375-85. Epub 2010/12/09. doi: 10.1074/jbc.M110.181784. PubMed PMID: 21138836; PubMed Central PMCID: PMC3057784.
21. Mizanur R M, et al., J Biol Chem. 2013; 288(33):24223-33. Epub 2013/06/18. doi: 10.1074/jbc.M113.451286. PubMed PMID: 23779108; PubMed Central PMCID: PMCPMC3745367.
22. Manglik A, et al., Annu Rev Pharmacol Toxicol. 2017; 57:19-37. Epub 2016/12/07. doi: 10.1146/annurev-pharmtox-010716-104710. PubMed PMID: 27959623; PubMed Central PMCID: PMCPMC5500200.
23. Lacy D B, et al., Nat Struct Biol. 1998; 5(10), p. 898-902.
24. Baldwin M R, et al., Protein ExprPurif 2004; 37(1):187-95. PubMed PMID: 15294297.
25. Kumaran D, et al., PLoS Pathog. 2008; 4(9):e1000165.Epub 2008/09/26. doi: 10.1371/journal.ppat.1000165. PubMed PMID: 18818739; PubMed Central PMCID: PMCPMC2533696.
26. Lawrence M C, et al., J Mol Biol. 1993; 234(4):946-50. doi: 10.1006/jmbi.1993.1648. PubMed PMID: 8263940.
27. Kuroda D, et al., Bioinformatics. 2016; 32(16):2451-6. Epub 20160419. doi: 10.1093/bioinformatics/btw197.PubMed PMID: 27153634; PubMed Central PMCID: PMCPMC4978935.
28. Zuniga J E, et al., Structure. 2008; 16(10):1588-97. doi: 10.1016/j.str.2008.07.011. PubMed PMID: 18940613; PubMed Central PMCID: PMCPMC2716802.
29. Zuniga J E, et al., PLoS One. 2010; 5(6):e11378. Epub 2010/06/30. doi: 10.1371/journal.pone.0011378. PubMed PMID: 20614028; PubMed Central PMCID: PMCPMC2894858.
30. Kumaran D, et al., Bioorg Med Chem. 2015; 23(22):7264-73. Epub 2015/10/17. doi: 10.1016/j.bmc.2015.10.024. PubMed PMID: 26522088.
31. Ahmed S A, et al., Protein J. 2008; 27(3):151-62. doi: 10.1007/s10930-007-9118-8. PubMed PMID: 18213512.
32. Chen S, et al., J Biol Chem. 2007; 282(13):9621-7. Epub 2007/01/23. doi: 10.1074/jbc.M611211200. PubMed PMID: 17244603.
33. Dong J, et al., Journal of Molecular Biology. 2010; 397(4):1106-18.doi: 10.1016/j.jmb.2010.01.070.
34. Chen S, et al., J Biol Chem. 2008; 283(30):21153-9. Epub 2008/05/29.doi: 10.1074/jbc.M800611200. PubMed PMID: 18511417; PubMed Central PMCID: PMCPMC2475686.
35. Agarwal R, et al., Nat Struct Mol Biol. 2009; 16(7):789-94. Epub 2009/06/21. doi: 10.1038/nsmb.1626. PubMed PMID: 19543288.
36. Sikorra S, et al., J Biol Chem. 2008; 283(30):21145-52.Epub 2008/05/29. doi: 10.1074/jbc.M800610200. PubMed PMID: 18511418; PubMed Central PMCID: PMCPMC3258937.
37. Chen S, et al., Biochemistry. 2012; 51(18):3941-7. Epub 2012/04/24. doi: 10.1021/bi3000098. PubMed PMID: 22510015; PubMed Central PMCID: PMCPMC3542401.
38. Sikorra S, et al., J Mol Biol. 2006; 357(2):574-82. Epub 2006/01/18. doi: 10.1016/j.jmb.2005.12.075. PubMed PMID: 16430921.
39. Fan Y, et al., Plos One. 2015; 10(8):e0135306. doi: 10.1371/journal.pone.0135306.PubMed PMID: Fan2015.
40. Lauwereys M, et al., Embo J. 1998; 17(13):3512-20. PubMed PMID: 9649422.
41. Lam K H, et al., Toxins. 2020; 12(10); PMID: 32992561.
42. Jaskiewicz J J, et al., Int J Parasitol. 2021; 51(9):761-75; PMID: 33774040; PubMed Central PMCID: PMC8373667.
43. Gu S, et al., Science. 2012; 335(6071):977-81. Epub 2012/03/01. 335/6071/977 [pii] https://doi.org/10.1126/science.1214270 PMID: 22363010.
44. Battye T G, et al., Acta Crystallogr D Biol Crystallogr. 67(Pt 4):271-81. Epub 2011/04/05. S0907444910048675 [pii] https://doi.org/10.1107/50907444910048675 PMID: 21460445; PubMed Central PMCID: PMC306972.
45. Kabsch W. XDS. Acta Crystallogr D Biol Crystallogr. 2010; 66(Pt 2):125-32. https://doi.org/10.1107/S0907444909047337 PMID: 20124692; PubMed Central PMCID: PMC2815665
46. McCoy A J, et al., J Appl Cryst. 2007; 40:658-74. PMID: 19461840.
47. Yao G, et al., Sci Rep. 2017; 7(1):7438. PMID: 28785006; PubMed Central PMCID: PMC5547058.
48. Biol Crystallogr. 2010; 66(Pt 4):486-501. https://doi.org/10.1107/S0907444910007493 PMID: 20383002; PubMed Central PMCID: PMC2852313.
49. Adams P D, et al., Acta Crystallogr D Biol Crystallogr. 2010; 66(Pt 2):213-21. Epub 2010/02/04. S0907444909052925 [pii] PMID: 20124702; PubMed Central PMCID: PMC2815670.
50. Winn M D, et al., Acta Crystallogr D Biol Crystallogr. 2011; 67(Pt 4):235-42. PMID: 21460441; PubMed Central PMCID: PMC3069738.
51. Brunger A T. Nature. 1992; 355(6359):472-5. Epub 1992/01/30. PMID: 18481394.
52. Chen V B, et al., Acta Crystallogr D Biol Crystallogr. 2010; 66(Pt 1):12-21. Epub 2010/01/09. S0907444909042073 [pii] PMID: 20057044; PubMed Central PMCID: PMC2803126.

1. A VHH polypeptide that specifically binds to a light chain (LC) protease domain of Botulinum neurotoxin (BoNT) serotype A (LC/A) or a LC/A-binding portion thereof, or to a light chain (LC) protease domain of Botulinum neurotoxin (BoNT) serotype B (LC/B), or a LC/B-binding portion thereof,
wherein the VHH polypeptide that binds to the LC/A neurotoxin comprises complementarity determining regions (CDRs), CDR1, CDR2 and CDR3, comprising amino acid sequences selected from:
CDR1: SGFTLDDYAIGWF as set forth in SEQ ID NO: 38; CDR2: SRSGDTYYP as set forth in SEQ ID NO: 39; and CDR3: DFPPVRPMCIQAAPKKR as set forth in SEQ ID NO: 40;
CDR1: SGADFSFYAMGWY as set forth in SEQ ID NO: 41; CDR2: NLNGVISYG as set forth in SEQ ID NO: 42; and CDR3: MRLYTRGSVRHPESW as set forth in SEQ ID NO: 43;
CDR1: TGRTLDYYALGWF as set forth in SEQ ID NO: 44; CDR2: NWLGGSTYYA as set forth in SEQ ID NO: 45; and CDR3: DFSIAYSGTYPPAYAEYDYDYW as set forth in SEQ ID NO: 46;
CDR1: SGSPLSIWVMGWY as set forth in SEQ ID NO: 47; CDR2: NLNGITSYG as set forth in SEQ ID NO: 48; and CDR3: EPLGPRGKKSGKEYW as set forth in SEQ ID NO: 49;
CDR1: TSENVFGIYGMAW as set forth in SEQ ID NO: 50; CDR2: ITSRGTAHYH as set forth in SEQ ID NO: 51; and CDR3: GPYW as set forth in SEQ ID NO: 52;
CDR1: SGFTFNRYVIRWY as set forth in SEQ ID NO: 53; CDR2: SRSGDSGRYV as set forth in SEQ ID NO: 54; and CDR3: LNLEDMEYW as set forth in SEQ ID NO: 55;
CDR1: SGNIFSIYYMGWY as set forth in SEQ ID NO: 56; CDR2: NSNGITNYG as set forth in SEQ ID NO: 57; and CDR3: GKLRRTTGWGLDDYW as set forth in SEQ ID NO: 58;
CDR1: SGFTLDEYAIGWF as set forth in SEQ ID NO: 59; CDR2: SSSASISYA as set forth in SEQ ID NO: 60; and CDR3: AFLACGPVAGWGTEYDYW as set forth in SEQ ID NO: 61;
CDR1: STTISDFYSMGWF as set forth in SEQ ID NO: 62; CDR2: RRGGDTKSG as set forth in SEQ ID NO: 63; and CDR3: NLQKSSDELGPYYW as set forth in SEQ ID NO: 64;
CDR1: SLLTLEYYAIGWF as set forth in SEQ ID NO: 65; CDR2: GSSGGSTVYI as set forth in SEQ ID NO: 66; and CDR3: DDLRCGRGWSSYFRGSW as set forth in SEQ ID NO: 67;
CDR1: SESVFEMYTVAWY as set forth in SEQ ID NO: 68; CDR2: TDEGRTNYA as set forth in SEQ ID NO: 69; and CDR3: EHDLGYYDYW as set forth in SEQ ID NO: 70;
CDR1: SGLTLDYYAIGWF as set forth in SEQ ID NO: 71; CDR2: SSGSSMSIHA as set forth in SEQ ID NO: 72; and CDR3: DDFTCGSRWSDWAHTFGFW as set forth in SEQ ID NO: 73; or
CDR1: SGGIFSTYIMGWY as set forth in SEQ ID NO: 74; CDR2: SNHTTDYA as set forth in SEQ ID NO: 75; and CDR3: DWMVGAWTAGDYGVDYW as set forth in SEQ ID NO: 76; and
wherein the VHH polypeptide that binds to the LC/B neurotoxin comprises complementarity determining regions (CDRs), CDR1, CDR2 and CDR3, comprising amino acid sequences selected from:
CDR1: SGLSFNWYDVG as set forth in SEQ ID NO: 77; CDR2: SRSSGGGSTY as set forth in SEQ ID NO: 78; and CDR3: AADWTGRAGFSVGYYRPDEYDY as set forth in SEQ ID NO: 79;
CDR1: SGFTLDSYAIG as set forth in SEQ ID NO: 80; CDR2: CMSSGDGSTY as set forth in SEQ ID NO: 81; and CDR3: AADGFDYCSAYVPGRGMNY as set forth in SEQ ID NO: 82;
CDR1: SGFTLDNYAVG as set forth in SEQ ID NO: 83; CDR2: CISSSDDNTD as set forth in SEQ ID NO: 84; and CDR3: AAESPTFGFSCTVATDPYDY as set forth in SEQ ID NO: 85;
CDR1: SGFTLDGYAAG as set forth in SEQ ID NO: 86; CDR2: WISSTDGSTY as set forth in SEQ ID NO: 87; and CDR3: TAGLGLDVSDYVYDY as set forth in SEQ ID NO: 88;
CDR1: SGFTLDYYGIG as set forth in SEQ ID NO: 89; CDR2: CITSGGLTN as set forth in SEQ ID NO: 90; and CDR3: AIDRVGVCAMEDFGS as set forth in SEQ ID NO: 91;
CDR1: SGRTFNYYAMA as set forth in SEQ ID NO: 92; CDR2: FINWSGDSTY as set forth in SEQ ID NO: 93; and CDR3: AAEFGTFSYLQGDDYSY as set forth in SEQ ID NO: 94;
CDR1: SGRSFSSYRMG as set forth in SEQ ID NO: 95; CDR2: GISWSGSSTW as set forth in SEQ ID NO: 96; and CDR3: AADGLGTDWSDAIWDY as set forth in SEQ ID NO: 97;
CDR1: SGRMFNEYRMG as set forth in SEQ ID NO: 98; CDR2: AINWGAQIPY as set forth in SEQ ID NO: 99; and CDR3: AADWGYGSSPHQDKEYDY as set forth in SEQ ID NO: 100;
CDR1: SGRTFSDYAMG as set forth in SEQ ID NO: 101; CDR2: AVDWSGSSRL as set forth in SEQ ID NO: 102; and CDR3: AAARNRWSSEISSYDY as set forth in SEQ ID NO: 103;
CDR1: SGRTFRRNTMG as set forth in SEQ ID NO: 104; CDR2: AISWSGDRTY as set forth in SEQ ID NO: 105; and CDR3: AADGTASVFNSYASADRNKYNY as set forth in SEQ ID NO: 106;
CDR1: SIRTFSTSTTA as set forth in SEQ ID NO: 107; CDR2: RISGSDPVTY as set forth in SEQ ID NO: 108; and CDR3: ATVRIKGGSEFSYHY as set forth in SEQ ID NO: 109;
CDR1: SESTFSINAIG as set forth in SEQ ID NO: 110; CDR2: HISTSGRTR as set forth in SEQ ID NO: 111; and CDR3: NAEGYSTWPEDRYLEL as set forth in SEQ ID NO: 112;
CDR1: SGRTFSSYRMGMG as set forth in SEQ ID NO: 113; CDR2: TVNWSGGTTY as set forth in SEQ ID NO: 114; and CDR3: AAGRGSESYTSSRYNY as set forth in SEQ ID NO: 115; or
CDR1: SGFTLDDYAIG as set forth in SEQ ID NO: 116; CDR2: CISIRDGRTH as set forth in SEQ ID NO: 117; and CDR3: AAGQRSMAYVCSNRFGS as set forth in SEQ ID NO: 118.

2. The polypeptide of claim 1, further comprising a framework region.

3. The polypeptide of claim 1, wherein the VHH polypeptide that binds to the LC/A neurotoxin comprises complementarity determining regions (CDRs), CDR1, CDR2 and CDR3, comprising amino acid sequences:
CDR1: SGADFSFYAMGWY as set forth in SEQ ID NO: 41; CDR2: NLNGVISYG as set forth in SEQ ID NO: 42; and CDR3: MRLYTRGSVRHPESW as set forth in SEQ ID NO: 43;
CDR1: TGRTLDYYALGWF as set forth in SEQ ID NO: 44; CDR2: NWLGGSTYYA as set forth in SEQ ID NO: 45; and CDR3: DFSIAYSGTYPPAYAEYDYDYW as set forth in SEQ ID NO: 46;
CDR1: SGFTFNRYVIRWY as set forth in SEQ ID NO: 53; CDR2: SRSGDSGRYV as set forth in SEQ ID NO: 54; and CDR3: LNLEDMEYW as set forth in SEQ ID NO: 55;
CDR1: SGNIFSIYYMGWY as set forth in SEQ ID NO: 56; CDR2: NSNGITNYG as set forth in SEQ ID NO: 57; and CDR3: GKLRRTTGWGLDDYW as set forth in SEQ ID NO: 58;
CDR1: SGFTLDEYAIGWF as set forth in SEQ ID NO: 59; CDR2: SSSASISYA as set forth in SEQ ID NO: 60; and CDR3: AFLACGPVAGWGTEYDYW as set forth in SEQ ID NO: 61; or
CDR1: STTISDFYSMGWF as set forth in SEQ ID NO: 62; CDR2: RRGGDTKSG as set forth in SEQ ID NO: 63; and CDR3: NLQKSSDELGPYYW as set forth in SEQ ID NO: 64; or
wherein the VHH polypeptide that binds to the LC/B neurotoxin comprises complementarity determining regions (CDRs), CDR1, CDR2 and CDR3, comprising amino acid sequences:
CDR1: SGRTFRRNTMG as set forth in SEQ ID NO: 104; CDR2: AISWSGDRTY as set forth in SEQ ID NO: 105; and CDR3: AADGTASVFNSYASADRNKYNY as set forth in SEQ ID NO: 106.

4. A VHH polypeptide that specifically binds to a light chain (LC) protease domain of Botulinum neurotoxin (BoNT) serotype A (LC/A) or a LC/A-binding portion thereof, or to a light chain (LC) protease domain of Botulinum neurotoxin (BoNT) serotype B (LC/B) or a LC/B-binding portion thereof, wherein the VHH polypeptide that binds to the LC/A toxin comprises an amino acid sequence having at least 85% sequence identity to an amino acid sequence of an LC/A-binding polypeptide selected from the group consisting of JPU-A1 as set forth in SEQ ID NO: 7; JPU-A5 as set forth in SEQ ID NO: 8; JPU-A11 as set forth in SEQ ID NO: 9; JPU-B5 as set forth in SEQ ID NO: 10; JPU-B9 as set forth in SEQ ID NO: 11; JPU-C1 as set forth in SEQ ID NO: 12; JPU-C10 as set forth in SEQ ID NO: 13; JPU-D12 as set forth in SEQ ID NO: 14; JPU-G3 as set forth in SEQ ID NO: 15; JPU-G7 as set forth in SEQ ID NO: 16; JPU-G11 as set forth in SEQ ID NO: 17; JPU-G12 as set forth in SEQ ID NO: 18; and JPU-H7 as set forth in SEQ ID NO: 19; or wherein the VHH polypeptide that binds to the LC/B toxin comprises an amino acid sequence having at least 85% sequence identity to an amino acid sequence of an LC/B toxin binding polypeptide selected from the group consisting of JND-A12 as set forth in SEQ ID NO: 24; JND-B4 as set forth in SEQ ID NO: 25; JND-C7 as set forth in SEQ ID NO: 26; JND-E4 as set forth in SEQ ID NO: 27; JND-E5 as set forth in SEQ ID NO: 28; JND-E9 as set forth in SEQ ID NO: 29; JND-F3 as set forth in SEQ ID NO: 30; JSG-B8 as set forth in SEQ ID NO: 31; JSG-B10 as set forth in SEQ ID NO: 32; JSG-C1 as set forth in SEQ ID NO: 33; JSG-F6 as set forth in SEQ ID NO: 34; JSG-G1 as set forth in SEQ ID NO: 35; JSG-G10 as set forth in SEQ ID NO: 36; and JSG-G11 as set forth in SEQ ID NO: 37.

5. The VHH polypeptide of claim 4, wherein the VHH polypeptide that binds to the LC/A toxin comprises an amino acid sequence having at least 85% sequence identity to an amino acid sequence of an LC/A toxin-binding polypeptide selected from the group consisting of JPU-A5 as set forth in SEQ ID NO: 8, JPU-A11 as set forth in SEQ ID NO: 9, JPU-C1 as set forth in SEQ ID NO: 12, JPU-C10 as set forth in SEQ ID NO: 13, JPU-D12 as set forth in SEQ ID NO: 14, or JPU-G3 as set forth in SEQ ID NO: 15; or wherein the VHH polypeptide that binds to the LC/B toxin comprises an amino acid sequence having at least 85% sequence identity to the amino acid sequence of LC/B toxin-binding VHH polypeptide JSG-C1 as set forth in SEQ ID NO: 33

6. A VHH polypeptide that specifically binds to a light chain (LC) protease domain of Botulinum neurotoxin (BoNT) serotype A (LC/A) or a LC/A-binding portion thereof, or to a light chain (LC) protease domain of Botulinum neurotoxin (BoNT) serotype B (LC/B) or a LC/B-binding portion thereof, wherein the VHH polypeptide that binds to LC/A comprises or consists of JPU-A1 as set forth in SEQ ID NO: 7; JPU-A5 as set forth in SEQ ID NO: 8; JPU-A11 as set forth in SEQ ID NO: 9; JPU-B5 as set forth in SEQ ID NO: 10; JPU-B9 as set forth in SEQ ID NO: 11; JPU-C1 as set forth in SEQ ID NO: 12; JPU-C10 as set forth in SEQ ID NO: 13; JPU-D12 as set forth in SEQ ID NO: 14; JPU-G3 as set forth in SEQ ID NO: 15; JPU-G7 as set forth in SEQ ID NO: 16; JPU-G11 as set forth in SEQ ID NO: 17; JPU-G12 as set forth in SEQ ID NO: 18; and JPU-H7 as set forth in SEQ ID NO: 19; or wherein the VHH polypeptide that binds to LC/B comprises or consists of JND-A12 as set forth in SEQ ID NO: 24; JND-B4 as set forth in SEQ ID NO: 25; JND-C7 as set forth in SEQ ID NO: 26; JND-E4 as set forth in SEQ ID NO: 27; JND-E5 as set forth in SEQ ID NO: 28; JND-E9 as set forth in SEQ ID NO: 29; JND-F3 as set forth in SEQ ID NO: 30; JSG-B8 as set forth in SEQ ID NO: 31; JSG-B10 as set forth in SEQ ID NO: 32; JSG-C1 as set forth in SEQ ID NO: 33; JSG-F6 as set forth in SEQ ID NO: 34; JSG-G1 as set forth in SEQ ID NO: 35; JSG-G10 as set forth in SEQ ID NO: 36; and JSG-G11 as set forth in SEQ ID NO: 37.

7. The VHH polypeptide of claim 6, wherein the LC/A-binding VHH polypeptide comprises or consists of JPU-A5 as set forth in SEQ ID NO: 8, JPU-A11 as set forth in SEQ ID NO: 9, JPU-C1 as set forth in SEQ ID NO: 12, JPU-C10 as set forth in SEQ ID NO: 13, JPU-D12 as set forth in SEQ ID NO: 14, or JPU-G3 as set forth in SEQ ID NO: 15; or wherein the LC/B-binding VHH polypeptide comprises or consists of JSG-C1 as set forth in SEQ ID NO: 33.

8. The polypeptide of claim 1, wherein the polypeptide inhibits and/or neutralizes BoNT/A LC/A protease function or activity or inhibits and/or neutralizes BoNT/B LC/B protease function or activity.

9. The polypeptide of claim 4, wherein conservative amino acid substitutions in the polypeptide comprise the at least 85% amino acid sequence identity.

10. The polypeptide of claim 1, which is a camelid-derived single domain anti-LC/A toxin or LC/B toxin VHH antibody.

11. The polypeptide of claim 1, comprising one or more epitope tag sequences specifically bindable by an anti-epitope tag antibody or binding portion thereof.

12. The polypeptide of claim 11, wherein the one or more epitope tag sequences comprises at least one of DELGPRLMGK as set forth in SEQ ID NO: 119 or GAPVPYPDPLEPR as set forth in SEQ ID NO: 120.

13. The polypeptide of claim 1, in the form of a dimer or multimer.

14. The polypeptide of claim 13, comprising two or more anti-LC/A toxin and/or anti-LC/B toxin VHH polypeptides, or LC/A-toxin or LC/B-toxin binding portions thereof, joined with one or more linker peptides.

15. The polypeptide of claim 14, wherein the one or more linker peptides is selected from GGGGS as set forth in SEQ ID NO: 121; GGGGSGGGGSGGGGS as set forth in SEQ ID NO: 122, or a functional portion thereof, EPKTPKPQGGGGSGGGGSGGGGSQGVQSQVQLVE as set forth in SEQ ID NO: 123; EPKTPKPQ as set forth in SEQ ID NO: 124; or a combination thereof.

16. The polypeptide of claim 14, wherein the polypeptide is dimeric and comprises (i) two anti-LC/A-toxin VHH polypeptides, same or different; (ii) two anti-LC/B-toxin VHH polypeptides, same or different; or (iii) a combination of an anti-LC/A-toxin VHH polypeptide and an anti-LC/B-toxin VHH polypeptide.

17. The polypeptide of claim 13, wherein the polypeptide is multimeric and comprises (i) at least three anti-LC/A toxin VHH polypeptides, same or different; (ii) at least three anti-LC/B toxin VHH polypeptides, same or different; or (iii) a combination of at least three anti-LC/A-toxin VHH polypeptides and anti-LC/B-toxin VHH polypeptides.

18. An isolated polynucleotide encoding the polypeptide of claim 1.

19. A vector comprising the isolated polynucleotide of claim 18; optionally, wherein the vector is a viral or a non-viral expression vector.

20. A host cell comprising the vector of claim 19.

21. A pharmaceutical composition comprising an effective amount of the polypeptide of claim 1 and a pharmaceutically acceptable excipient, carrier, or diluent.

22. A pharmaceutical composition comprising an effective amount of the polypeptide of claim 4 and a pharmaceutically acceptable excipient, carrier, or diluent.

23. A method of treating botulism or intoxication associated with activity of Botulinum LC/A and/or LC/B neurotoxins, and/or the symptoms thereof, the method comprising administering to a subject in need thereof an effective amount of the polypeptide of claim 1, or a polynucleotide encoding the polypeptide, or a pharmaceutical composition thereof, thereby treating botulism or infection by Botulinum microorganisms of serotype A and/or B, and/or the symptoms thereof in the subject.

24. A method of treating botulism or intoxication associated with activity of Botulinum LC/A and/or LC/B neurotoxins, and/or the symptoms thereof, the method comprising administering to a subject in need thereof an effective amount of the polypeptide of claim 4, or a polynucleotide encoding the polypeptide, or a pharmaceutical composition thereof, thereby treating botulism or infection by Botulinum microorganisms of serotype A and/or B, and/or the symptoms thereof in the subject.

25. A method of reducing the severity of botulism or intoxication associated with activity of Botulinum LC/A and/or LC/B neurotoxins, and/or the symptoms thereof, in a subject who has, who is susceptible to, or who is at risk of having, botulism or intoxication by the Botulinum microorganisms, the method comprising administering to a subject in need thereof an effective amount of the polypeptide of claim 1, or a polynucleotide encoding the polypeptide, or a pharmaceutical composition thereof, thereby reducing the severity of botulism or intoxication by the LC/A and/or LC/B neurotoxin-producing Botulinum microorganisms and/or the symptoms thereof in the subject.

26. A method of reducing the severity of botulism or intoxication associated with activity of Botulinum LC/A and/or LC/B neurotoxins, and/or the symptoms thereof in a subject who has, who is susceptible to, or who is at risk of having, botulism or intoxication by the Botulinum neurotoxins, the method comprising administering to a subject in need thereof an effective amount of the polypeptide of claim 4, or a polynucleotide encoding the polypeptide, or a pharmaceutical composition thereof, thereby reducing the severity of botulism or intoxication by the LC/A and/or LC/B neurotoxin-producing Botulinum microorganisms and/or the symptoms thereof in the subject.

27. The method of 23, further comprising administering to the subject an anti-epitope tag antibody that specifically binds to an epitope tag, if present, and facilitates clearance of a complex of LC/A and/or LC/B bound to the anti-LC/A and/or anti-LC/B VHH polypeptide from the subject.

28. The method of 24, further comprising administering to the subject an anti-epitope tag antibody that specifically binds to an epitope tag, if present, and facilitates clearance of a complex of LC/A and/or LC/B bound to the anti-LC/A and/or anti-LC/B VHH polypeptide from the subject.

29. The method of claim 23, wherein the VHH polypeptide provides post-exposure or late post-exposure therapeutic treatment for patients suffering from botulism.

30. The method of claim 24, wherein the VHH polypeptide provides post-exposure or late post-exposure therapeutic treatment for patients suffering from botulism.

31. A kit comprising the polypeptide of claim 1, or a pharmaceutical composition thereof, for treating or protecting against disease or intoxication and/or the symptoms thereof caused by Botulinum LC/A toxin protease and/or Botulinum LC/B toxin protease; and optionally comprising instructions for use.

32. A method of detecting botulinum protease LC/A and/or LC/B in a sample, the method comprising contacting the sample with one or more VHH polypeptides of claim 1 under conditions for detecting the binding of the VHH polypeptide to the LC/A and/or the LC/B in the sample.
